SEQUENCE LISTING (21 sequences)

SEQ ID NO: 1 - protein, 50 aa, Artificial Sequence
  Description of Artificial Sequence Synthetic polypeptide
  SITE (1)..(50): This sequence may encompass 1-10 “Gly Gly Gly Gly Ser” repeating units
GGGGSGGGGSGGGGSGGGGSGGGGSGGGGSGGGGSGGGGSGGGGSGGGGS

SEQ ID NO: 2 - protein, 5 aa, Artificial Sequence
  Description of Artificial Sequence Synthetic peptide
  MOD_RES (3)..(3): Any amino acid
LPXTG

SEQ ID NO: 3 - protein, 20 aa, Homo sapiens
MSRSVALAVLALLSLSGLEA

SEQ ID NO: 4 - protein, 24 aa, Homo sapiens
VGIIAGLVLLGAVITGAVVAAVMW

SEQ ID NO: 5 - protein, 5 aa, Unknown
  Description of Unknown Enterokinase cleavage site sequence
DDDDK

SEQ ID NO: 6 - protein, 4 aa, Unknown
  Description of Unknown Factor Xa cleavage site sequence
IEGR

SEQ ID NO: 7 - protein, 6 aa, Unknown
  Description of Unknown Thrombin cleavage site sequence
LVPRGS

SEQ ID NO: 8 - protein, 8 aa, Unknown
  Description of Unknown Renin cleavage site sequence
HPFHLVIH

SEQ ID NO: 9 - protein, 4 aa, Unknown
  Description of Unknown Caspase protease cleavage site sequence
DEVD

SEQ ID NO: 10 - DNA, 20 bp, Homo sapiens
actcacgctggatagcctcc

SEQ ID NO: 11 - DNA, 20 bp, Homo sapiens
gagtagcgcgagcacagcta

SEQ ID NO: 12 - DNA, 20 bp, Homo sapiens
cagtaagtcaacttcaatgt

SEQ ID NO: 13 - DNA, 19 bp, Homo sapiens
gccgcctcccacttgcgct

SEQ ID NO: 14 - DNA, 20 bp, Homo sapiens
cacatgcagcccacgagccg

SEQ ID NO: 15 - DNA, 20 bp, Homo sapiens
atccctaaatatggtgtccc

SEQ ID NO: 16 - DNA, 20 bp, Homo sapiens
tccctaaatatggtgtccct

SEQ ID NO: 17 - DNA, 20 bp, Homo sapiens
gtgatccgggtatgggcagt

SEQ ID NO: 18 - DNA, 20 bp, Homo sapiens
tgatccgggtatgggcagtg

SEQ ID NO: 19 - protein, 475 aa, Artificial Sequence
  Description of Artificial Sequence Synthetic polypeptide
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 20 - DNA, 18 bp, Homo sapiens
tccatctggtcatagaag

SEQ ID NO: 21 - protein, 25 aa, Artificial Sequence
  Description of Artificial Sequence Synthetic peptide
DSSLQARLFPGLTIKIQRSNGLIHS

(19) United States
(12) Patent Application Publication
(10) Pub. No.: US 2024/0118285 A1
(43) Pub. Date: Apr. 11, 2024

(54) COMPOSITIONS AND METHODS FOR T-CELL RECEPTOR IDENTIFICATION

(71) Applicant: RootPath Genomics, Inc., Watertown, MA (US)

(72) Inventor: Xi CHEN, Newton, MA (US)

(21) Appl. No.: 18/032,527

(22) PCT Filed: Oct. 22, 2021
(86) PCT No.: PCT/US2021/056208

§ 371 Date: Apr. 18, 2023

Related U.S. Application Data
(60) Provisional application No. 63/128,274, filed on Dec. 21, 2020.
(60) Provisional application No. 63/104,624, filed on Oct. 23, 2020.

(51) Int. Cl. G01N 33/574

(52) U.S. Cl. CPC G01N 33/57492; C12N 2503/00; G01N 2333/70539

(57) ABSTRACT

The present disclosure provides compositions and methods for identifying antigen-reactive T-cell receptors (TCRs). A cancer cell line expressing an endogenous antigen in complex with an exogenous MHC molecule derived from a subject (e.g., a cancer patient) can be used to screen for TCRs recognizing the endogenous antigen in complex with the exogenous MHC molecule.

### CROSS-REFERENCE

This application claims the benefit of U.S. Provisional Patent Application No. 63/104,624, filed Oct. 23, 2020, and U.S. Provisional Patent Application No. 63/128,274, filed Dec. 21, 2020, each of which is incorporated herein by reference in its entirety.

### BACKGROUND OF THE INVENTION

The T-cell receptor (TCR) is responsible for the recognition of the antigen-major histocompatibility complex, leading to the initiation of an inflammatory response. Many T cell subsets exist, including cytotoxic T cells and helper T cells. Cytotoxic T cells (also known as CD8+ T cells) kill abnormal cells, for example virus-infected or tumor cells. Helper T cells (also known as CD4+ T cells) aid in the activation and maturation of other immune cells. Both cytotoxic and helper T cells carry out their function subsequent to the recognition of specific target antigens which triggers their respective responses. The antigen specificity of a T cell can be defined by the TCR expressed on the surface of the T cell. T cell receptors are heterodimer proteins composed of two polypeptide chains, most commonly an alpha and beta chain, but a minority of T cells can express a gamma and delta chain. The specific amino acid sequence of the TCR and the resultant three-dimensional structure defines the TCR antigen specificity and affinity. The amino acid and coding DNA sequences of the TCR chains for any individual T cell are almost always unique or at very low abundance in an organism's entire TCR repertoire, since there are a vast number of possible TCR sequences. This large sequence diversity is achieved during T cell development through a number of cellular mechanisms and may be a critical aspect of the immune system's ability to respond to a huge variety of potential antigens.

Analyzing the TCR repertoire may help to gain a better understanding of the immune system features and of the aetiology and progression of diseases, in particular those with unknown antigenic triggers.

### SUMMARY OF THE INVENTION

Recognized herein is a need to develop efficient ways for screening or evaluating antigen-reactive T-cell receptors (TCRs) or T cells. The compositions and methods can be used in various situations including when primary tumor sample of a subject cannot be reliably obtained in sufficient quality and/or quantity. The compositions and methods provided herein can be non-invasive. The compositions and methods provided herein, in some aspects, use cancer cell lines for antigen-reactive TCR or antigen-reactive T cell identification.

In an aspect, the present disclosure provides a method for identifying an antigen-reactive cell that recognizes an endogenous antigen of a cancer cell line in complex with an MHC molecule expressed by a subject, comprising: (a) providing a cell that is a cancer cell line expressing an endogenous antigen in complex with an exogenous MHC molecule, wherein the exogenous MHC molecule is the MHC molecule expressed by the subject or derived from the subject; (b) contacting the cancer cell line with a first plurality of TCR-expressing cells, wherein the first plurality of TCR-expressing cells or a subset of the first plurality of TCR-expressing cells is activated by the endogenous antigen in complex with the exogenous MHC of the cancer cell line; and (c) subsequent to contacting in (b), identifying the subset of the first plurality of TCR-expressing cells, thereby identifying the antigen-reactive cell that recognizes the endogenous antigen of the cancer cell line. In some embodiments, identifying in (c) comprises enriching or selecting the subset of the first plurality of TCR-expressing cells.

In some embodiments, the exogenous MHC molecule is exogenous to the cancer cell line. In some embodiments, the method further comprises, in (a), providing a non-cancer cell expressing an additional endogenous antigen in complex with an exogenous MHC molecule, wherein the exogenous MHC molecule is derived from the same subject. In some embodiments, the method further comprises, in (b), contacting the non-cancer cell with a second plurality of TCR-expressing cells, and wherein a subset of the second plurality of TCR-expressing cells is activated by the additional endogenous antigen in complex with the exogenous MHC of the non-cancer cell. In some embodiments, the additional endogenous antigen is the same as or different from the endogenous antigen expressed by the cancer cell line. In some embodiments, the non-cancer cell (i) does not express the endogenous antigen expressed by the cancer cell line, (ii) expresses the endogenous antigen expressed by the cancer cell line at a lower level, or (iii) expresses the endogenous antigen expressed by the cancer cell line, but does not present the endogenous antigen expressed by the cancer cell line. In some embodiments, the first plurality and the second plurality of TCR-expressing cells are derived from a same sample. In some embodiments, the first plurality and the second plurality of TCR-expressing cells express a same TCR. In some embodiments, the first plurality or the second plurality of TCR-expressing cells expresses different TCRs. In some embodiments, the method further comprises, in (c), identifying the subset of the second plurality of TCR-expressing cells. In some embodiments, identifying comprises selecting the subset of the first plurality of TCR-expressing cells and/or the subset of the second plurality of TCR-expressing cells based on a marker. In some embodiments, selecting the subset of the first plurality of TCR-expressing cells and/or the subset of the second plurality of TCR-expressing cells comprises using fluorescence activated cell sorting (FACS) or magnetic activated cell sorting (MACS) based on the marker.

In some embodiments, the method further comprises identifying a TCR that is expressed in the subset of the first plurality of TCR-expressing cells. In some embodiments, the method further comprises identifying a TCR that is expressed in the subset of the first plurality of TCR-expressing cells, but not in the subset of the second plurality of TCR-expressing cells.

In some embodiments, the method further comprises identifying a TCR of a cell in the subset of the first plurality of TCR-expressing cells that is activated by the endogenous antigen in complex with the exogenous MHC of the cancer cell line, and that is in a cell in the second plurality of TCR-expressing cells that is not activated by the additional endogenous antigen in complex with the exogenous MHC of the non-cancer cell.

In some embodiments, the non-cancer cell is a stem cell or a primary cell. In some embodiments, the stem cell is an induced pluripotent stem cell (iPSC). In some embodiments, the non-cancer cell is an differentiated iPSC. In some embodiments, the non-cancer cell expresses an autoimmune regulator (AIRE). In some embodiments, an endogenous MHC molecule of the cancer cell line or the non-cancer cell is inactivated (e.g., knocked down, or knocked out). In some embodiments, the cancer cell line or non-cancer cell is null for an endogenous MHC molecule. In some embodiments, the cancer cell line or non-cancer cell is null for all endogenous MHC molecules. In some embodiments, the endogenous MHC molecule comprises a MHC class I molecule, a MHC class II molecule, or a combination thereof. In some embodiments, the MHC class I molecule comprises HLA-A, HLA-B, HLA-C, or any combination thereof. In some embodiments, an alpha chain of the MHC class I molecule (MHC-I alpha) is inactivated. In some embodiments, a gene encoding the alpha chain of the MHC class I molecule is inactivated. In some embodiments, a beta-2-microglobulin (B2M) of the MHC class I molecule is inactivated. In some embodiments, a gene encoding the B2M of the MHC class I molecule is inactivated. In some embodiments, the MHC class II molecule comprises HLA-DP, HLA-DM, HLA-DOA, HLA-DOB, HLA-DQ, HLA-DR, or any combination thereof.

In some embodiments, an alpha chain or a beta chain of the MHC class II molecule is inactivated. In some embodiments, a gene encoding the alpha chain or the beta chain of the MHC class II molecule is inactivated. In some embodiments, a gene regulating transcription of the MHC class II molecule is inactivated. In some embodiments, the gene is CIITA.

In some embodiments, the exogenous MHC molecule of the cancer cell line or the non-cancer cell comprises a MHC class I molecule, a MHC class II molecule, or a combination thereof, derived from the subject. In some embodiments, the MHC class I molecule comprises HLA-A, HLA-B, HLA-C, or any combination thereof. In some embodiments, the MHC class II molecule comprises HLA-DP, HLA-DM, HLA-DOA, HLA-DOB, HLA-DQ, HLA-DR, or any combination thereof. In some embodiments, the exogenous MHC molecule comprises an MHC-I alpha derived from the subject and an endogenous B2M. In some embodiments, the exogenous MHC molecule comprises both an MHC-I alpha and a B2M derived from the subject. In some embodiments, the exogenous MHC molecule is a fusion protein of the MHC-I alpha and the B2M (B2M-MHC-I-alpha fusion). In some embodiments, the MHC-I alpha and the B2M is linked by a linker. In some embodiments, the linker is (G₄S)n, wherein G is glycine, S is serine, and n is an integer from 1 to 10. In some embodiments, the exogenous MHC molecule comprises an MHC-II alpha and an MHC-II beta derived from the subject.

In some embodiments, the first plurality of TCR-expressing cells is isolated from the same subject. In some embodiments, the first plurality of TCR-expressing cells comprises a primary T cell. In some embodiments, the primary T cell is a tumor-infiltrating T cell. In some embodiments, the primary T cell is a peripheral T cell. In some embodiments, the peripheral T cell is a tumor-experienced T cell. In some embodiments, the peripheral T cell is a PD-1+ T cell. In some embodiments, the primary T cell is a CD4+ T cell, a CD8+ T cell, or a combination thereof. In some embodiments, the primary T cell is a cytotoxic T cell, a memory T cell, a national killer T cell, an alpha beta T cell, a gamma delta T cell, or any combination thereof. In some embodiments, the first plurality of TCR-expressing cells comprises an engineered cell. In some embodiments, the engineered cell expresses an exogenous TCR. In some embodiments, the exogenous TCR is derived from a primary T cell isolated from the same subject.

In some embodiments, the method further comprises, prior to (a), isolating a primary cancer cell or a tumor sample from the subject.

In some embodiments, the method further comprises conducting transcriptomic or genomic analysis of the primary cancer cell or the tumor sample and cancer cell lines to identify the cancer cell line having a gene expression profile, a transcriptomic profile or a genomic alteration that resembles a primary cancer cell or the tumor sample isolated from the subject. In some embodiments, a correlation coefficient of the gene expression profile, the transcriptomic profile or the genomic alteration between the cancer cell line and the primary cancer cell or the tumor sample is equal to or greater than about 0.1.

In some embodiments, the method further comprises, in (c), identifying a TCR of the subset. In some embodiments, the method further comprises identifying a sequence of a TCR expressed by the antigen-reactive cell. In some embodiments, identifying the sequence of the TCR comprises sequencing a TCR repertoire of the subset of the first plurality of TCR-expressing cells. In some embodiments, identifying the sequence of the TCR further comprises sequencing a TCR repertoire of the first plurality of TCR-expressing cells prior to contacting with the cancer cell line. In some embodiments, a frequency of the TCR expressed by the antigen-reactive cell in the subset is higher than a frequency of the TCR expressed by the antigen-reactive cell in the first plurality.

In some embodiments, the method further comprises administering the antigen-reactive cell or a cell comprising a sequence encoding the TCR of the antigen-reactive cell into the subject.

In some embodiments, the first plurality of TCR-expressing cells expresses a plurality of TCRs comprising at least 10 different cognate pairs derived from the same subject. In some embodiments, the plurality of TCRs comprises V regions from a plurality of V genes.

In some embodiments, the cell that is a cancer cell line comprises at least about 50, 100, 1,000 or more cells.

In some embodiments, the method further comprises, prior to (b), killing the cancer cell line. In some embodiments, killing comprising irradiating or treating the cancer cell line with a chemical compound. In some embodiments, the chemical compound is a cytotoxic compound. In some embodiments, the cytotoxic compound is cis-platin, cyclophosphamide, nitrogen mustard, trimethylene thiophosphoramide, carmustine, busulfan, chlorambucil, belustine, uracil mustard, chlomaphazin, dacabazine, cytosine arabinoside, fluorouracil, methotrexate, mercaptopuirine, azathioprime, procarbazine, doxorubicin, bleomycin, dactinomycin, daunorubicin, mithramycin, mitomycin, mytomycin C, daunomycin, or any combination thereof.

In another aspect, the present disclosure provides a method for identifying an antigen-reactive cell that recognizes an antigen in complex with an MHC molecule expressed by a subject, comprising: (a) providing a cancer cell line expressing an antigen in complex with an exogenous MHC molecule, wherein the exogenous MHC molecule is the MHC molecule expressed by the subject or derived from the subject; (b) contacting the cancer cell line with a plurality of engineered cells expressing a plurality of TCRs comprising at least 10 different cognate pairs derived from the same subject, and wherein a subset of the plurality of engineered cells is activated by the antigen in complex with the exogenous MHC of the cancer cell line; and (c) subsequent to contacting in (b), identifying the subset of the plurality of engineered cells, thereby identifying the antigen-reactive cell.

In some embodiments, the antigen is endogenous to the cancer cell line. In some embodiments, the cancer cell line does not express an exogenous antigen or does not present an exogenous antigen. In some embodiments, the antigen is a tumor-associated antigen (TAA) or a tumor-specific antigen (TSA). In some embodiments, the cancer cell line is not derived from the same subject. In some embodiments, the cancer cell line has a transcriptomic profile or genomic alteration that resembles a primary cancer cell isolated from the subject. In some embodiments, the plurality of TCRs are exogenous to the plurality of engineered cells. In some embodiments, an endogenous MHC molecule of the cancer cell line is inactivated (e.g., knocked down, or knocked out). In some embodiments, the endogenous MHC molecule comprises a MHC class I molecule, a MHC class II molecule, or a combination thereof. In some embodiments, the MHC class I molecule comprises HLA-A, HLA-B, HLA-C, or any combination thereof. In some embodiments, an alpha chain of the MHC class I molecule (MHC-I alpha) is inactivated. In some embodiments, a gene encoding the alpha chain of the MHC class I molecule is inactivated. In some embodiments, an beta-2-microglobulin (B2M) of the MHC class I molecule is inactivated. In some embodiments, a gene encoding the B2M of the MHC class I molecule is inactivated. In some embodiments, the MHC class II molecule comprises HLA-DP, HLA-DM, HLA-DOA, HLA-DOB, HLA-DQ, HLA-DR, or any combination thereof. In some embodiments, an alpha chain or a beta chain of the MHC class II molecule is inactivated. In some embodiments, a gene encoding the alpha chain or the beta chain of the MHC class II molecule is inactivated. In some embodiments, a gene regulating transcription of the MHC class II molecule is inactivated. In some embodiments, the gene is CIITA.

In some embodiments, the exogenous MHC molecule comprises a MHC class I molecule, a MHC class II molecule, or a combination thereof, derived from the subject. In some embodiments, the MHC class I molecule comprises HLA-A, HLA-B, HLA-C, or any combination thereof. In some embodiments, the MHC class II molecule comprises HLA-DP, HLA-DM, HLA-DOA, HLA-DOB, HLA-DQ, HLA-DR, or any combination thereof. In some embodiments, the exogenous MHC molecule comprises an MHC-I alpha derived from the subject and an endogenous B2M. In some embodiments, the exogenous MHC molecule comprises both an MHC-I alpha and a B2M derived from the subject. In some embodiments, the exogenous MHC molecule is a fusion protein of the MHC-I alpha and the B2M (B2M-MHC-I-alpha fusion). In some embodiments, the MHC-I alpha and the B2M is linked by a linker. In some embodiments, the linker is (G₄S)ₙ, wherein G is glycine, S is serine, and n is an integer from 1 to 10. In some embodiments, the exogenous MHC molecule comprises an MHC-II alpha and an MHC-II beta derived from the subject.

In some embodiments, the plurality of TCRs comprises V regions from a plurality of V genes. In some embodiments, the plurality of TCRs is derived from a primary cell isolated from the same subject. In some embodiments, the primary cell is a T cell. In some embodiments, the T cell is a tumor-infiltrating T cell. In some embodiments, the T cell is a peripheral T cell. In some embodiments, the peripheral T cell is a tumor-experienced T cell. In some embodiments, the peripheral T cell is a PD-1+ T cell. In some embodiments, the T cell is a CD4+ T cell, a CD8+ T cell, or a combination thereof. In some embodiments, the T cell is a cytotoxic T cell, a memory T cell, a national killer T cell, an alpha beta T cell, a gamma delta T cell, or any combination thereof. In some embodiments, identifying in (c) comprises enriching or selecting the subset of the plurality of engineered cells. In some embodiments, identifying in (c) comprises selecting the subset of the plurality of engineered cells based on a marker. In some embodiments, selecting comprises using FACS or MACS based on the marker. In some embodiments, the marker is a reporter protein. In some embodiments, the reporter protein is a fluorescent protein.

In some embodiments, the marker is a cell surface protein, an intracellular protein or a secreted protein. In some embodiments, the marker is the intracellular protein or the secreted protein, and wherein the method further comprises, prior to selecting, fixing and/or permeabilizing the plurality of engineered cells. In some embodiments, the method further comprises contacting the plurality of engineered cells with a Golgi blocker. In some embodiments, the secreted protein is a cytokine. In some embodiments, the cytokine is IFN-γ, TNF-alpha, IL-17A, IL-2, IL-3, IL-4, GM-CSF, IL-10, IL-13, granzyme B, perforin, or a combination thereof. In some embodiments, the cell surface protein is CD39, CD69, CD103, CD25, PD-1, TIM-3, OX-40, 4-1BB, CD137, CD3, CD28, CD4, CD8, CD45RA, CD45RO, GITR, FoxP3, or a combination thereof.

In some embodiments, the method further comprises identifying a TCR expressed by the antigen-reactive cell. In some embodiments, identifying the TCR comprises sequencing a TCR repertoire of the subset of the plurality of engineered cells. In some embodiments, the method further comprises administering the antigen-reactive cell or a cell comprising a sequence encoding the TCR of the antigen-reactive cell into the subject. In some embodiments, the method further comprises, prior to (a), isolating a primary cancer cell from the subject. In some embodiments, the method further comprises conducting transcriptomic or genomic analysis of the primary cancer cell and cancer cell lines to identify the cancer cell line having a transcriptomic profile or genomic alteration that resembles a primary cancer cell isolated from the subject.

In another aspect, the present disclosure provides a pharmaceutical composition comprising an antigen-reactive cell or a cell comprising a sequence encoding a TCR of the antigen-reactive cell identified by a method described herein.

In another aspect, the present disclosure provides a composition for identifying an antigen-reactive cell that recognizes an endogenous antigen of a cancer cell line in complex with an MHC molecule expressed by a subject, comprising: a cell that is a cancer cell line expressing an endogenous antigen in complex with an exogenous MHC molecule, wherein the exogenous MHC molecule is the MHC molecule expressed by the subject or derived from the subject; and a T cell expressing a natively paired TCR derived from the subject, wherein a gene expression profile, a transcriptomic profile or a genomic alternation of the cancer cell line resembles that of a cancer cell from the subject.

In some embodiments, a correlation coefficient of the gene expression profile, the transcriptomic profile or the genomic alteration between the cancer cell line and the primary cancer cell or the tumor sample is equal to or greater than about 0.1. In some embodiments, the cancer cell line does not comprise or present an exogenous antigen. In some embodiments, an endogenous MHC molecule of the cancer cell line is inactivated. In some embodiments, the cancer cell line is null for an endogenous MHC molecule. In some embodiments, the cancer cell line is null for all endogenous MHC molecules. In some embodiments, the endogenous MHC molecule comprises a MHC class I molecule, a MHC class II molecule, or a combination thereof. In some embodiments, the MHC class I molecule comprises HLA-A, HLA-B, HLA-C, or any combination thereof. In some embodiments, an alpha chain of the MHC class I molecule (MHC-I alpha) is inactivated. In some embodiments, a gene encoding the alpha chain of the MHC class I molecule is inactivated. In some embodiments, a beta-2-microglobulin (B2M) of the MHC class I molecule is inactivated. In some embodiments, a gene encoding the B2M of the MHC class I molecule is inactivated. In some embodiments, the MHC class II molecule comprises HLA-DP, HLA-DM, HLA-DOA, HLA-DOB, HLA-DQ, HLA-DR, or any combination thereof. In some embodiments, an alpha chain or a beta chain of the MHC class II molecule is inactivated. In some embodiments, a gene encoding the alpha chain or the beta chain of the MHC class II molecule is inactivated. In some embodiments, a gene regulating transcription of the MHC class II molecule is inactivated. In some embodiments, the gene is CIITA. In some embodiments, the exogenous MHC molecule of the cancer cell line comprises a MHC class I molecule, a MHC class II molecule, or a combination thereof, derived from the subject. In some embodiments, the MHC class I molecule comprises HLA-A, HLA-B, HLA-C, or any combination thereof. In some embodiments, the MHC class II molecule comprises HLA-DP, HLA-DM, HLA-DOA, HLA-DOB, HLA-DQ, HLA-DR, or any combination thereof. In some embodiments, the exogenous MHC molecule comprises an MHC-I alpha derived from the subject and an endogenous B2M. In some embodiments, the exogenous MHC molecule comprises both an MHC-I alpha and a B2M derived from the subject. In some embodiments, the exogenous MHC molecule is a fusion protein of the MHC-I alpha and the B2M (B2M-MHC-I-alpha fusion). In some embodiments, the MHC-I alpha and the B2M is linked by a linker. In some embodiments, the linker is (G₄S)n, wherein G is glycine, S is serine, and n is an integer from 1 to 10. In some embodiments, the exogenous MHC molecule comprises an MHC-II alpha and an MHC-II beta derived from the subject. In some embodiments, the T cell are a plurality of T cells, each expressing a different natively paired TCR derived from the subject. In some embodiments, the plurality of T cells comprise at least 10 different natively paired TCRs derived from the subject.

In another aspect, the present disclosure provides a method for evaluating an anti-cancer activity of a TCR-expressing cell, comprising: (a) providing a plurality of cells, wherein the plurality of cells is derived from a cancer line and express an endogenous antigen in complex with an exogenous MHC molecule, wherein the exogenous MHC molecule is an MHC molecule expressed by a subject or derived from the subject; (b) contacting the plurality of cells with a plurality of TCR-expressing cells expressing a plurality of TCRs derived from the same subject, wherein the plurality of TCRs or a fraction thereof recognizes the endogenous antigen in complex with the exogenous MHC molecule of the plurality of cells or a fraction thereof; and (c) subsequent to contacting in (b), quantifying (i) the fraction of the plurality of cells that are recognized by the plurality of TCR-expressing cells or a fraction thereof, (ii) the fraction of the plurality of TCR-expressing cells that recognize the plurality of cells or a fraction thereof, and/or (iii) a cytokine secreted by the plurality of TCR-expressing cells or a fraction thereof. In some embodiments, an endogenous MHC molecule of the plurality of cells is inactivated. In some embodiments, the plurality of cells is null for an endogenous MHC molecule. In some embodiments, the plurality of cells is null for all endogenous MHC molecules. In some embodiments, the endogenous MHC molecule comprises a MHC class I molecule, a MHC class II molecule, or a combination thereof. In some embodiments, an alpha chain of the MHC class I molecule (MHC-I alpha) is inactivated. In some embodiments, a gene encoding the alpha chain of the MHC class I molecule is inactivated. In some embodiments, a beta-2-microglobulin (B2M) of the MHC class I molecule is inactivated. In some embodiments, a gene encoding the B2M of the MHC class I molecule is inactivated. In some embodiments, an alpha chain or a beta chain of the MHC class II molecule is inactivated. In some embodiments, a gene encoding the alpha chain or the beta chain of the MHC class II molecule is inactivated. In some embodiments, a gene regulating transcription of the MHC class II molecule is inactivated. In some embodiments, the exogenous MHC molecule of the plurality of cells comprises a MHC class I molecule, a MHC class II molecule, or a combination thereof, derived from the subject. In some embodiments, the exogenous MHC molecule comprises an MHC-I alpha derived from the subject and an endogenous B2M. In some embodiments, the exogenous MHC molecule comprises both an MHC-I alpha and a B2M derived from the subject. In some embodiments, the exogenous MHC molecule is a fusion protein of the MHC-I alpha and the B2M (B2M-MHC-I-alpha fusion). In some embodiments, the MHC-I alpha and the B2M is linked by a linker. In some embodiments, the exogenous MHC molecule comprises an MHC-II alpha and an MHC-II beta derived from the subject. In some embodiments, the plurality of TCR-expressing cells is isolated from the same subject. In some embodiments, the plurality of TCR-expressing cells comprises a primary T cell. In some embodiments, the plurality of TCR-expressing cells comprises an engineered cell. In some embodiments, the engineered cell expresses an exogenous TCR. In some embodiments, quantifying the fraction of (i) or (ii) comprising using a flow cytometry based method. In some embodiments, the flow cytometry based method is FACS or MACS. In some embodiments, quantifying the fraction of (i) comprising determining an amount of lactate dehydrogenase released from the fraction.

In another aspect, the present disclosure provides a composition comprising a panel of MHC-engineered cancer cell lines derived from a same cancer type, comprising: a first sub-panel comprising at least two MHC-engineered cancer cell lines derived from a same first parental cancer cell line; and a second sub-panel comprising at least two MHC-engineered cancer cell lines derived from a same second parental cancer cell line; and wherein the at least two MHC-engineered cancer cell lines of the first sub-panel or the second sub-panel expresses a different exogenous MHC molecule.

In some embodiments, the at least two MHC-engineered cancer cell lines of the first sub-panel or the second sub-panel do not express a same exogenous and/or endogenous MHC molecule. In some embodiments, the at least two MHC-engineered cancer cell lines comprise at least about 10, 20, 30, 40, 50, 60, 70, 80, 90, 100, or more MHC-engineered cancer cell lines, each MHC-engineered cancer cell line expressing a different exogenous MHC molecule. In some embodiments, the first parental cancer cell line and the second parental cancer cell line are different. In some embodiments, an endogenous MHC molecule of the at least two MHC-engineered cancer cell lines of the first sub-panel or the second sub-panel is inactivated. In some embodiments, the exogenous MHC molecule is expressed by a subject or derived from the subject. In some embodiments, the composition further comprises a plurality of T cells. In some embodiments, each cancer cell line of the at least two MHC-engineered cancer cell lines in the first sub-panel or the second sub-panel is mixed with the plurality of T cells. In some embodiments, the plurality of T cells comprises at least two different natively paired TCRs. In some embodiments, the natively paired TCRs are derived from the same subject. In some embodiments, the panel of MHC-engineered cancer cell lines is derived from bladder cancer, bone cancer, brain cancer, breast cancer, colon cancer, ovarian cancer, head/neck cancer, leukemia, lymphoma, liver cancer, lung cancer, melanoma, pancreatic cancer, soft-tissue sarcoma, or stomach cancer.

In another aspect, the present disclosure provides a method for identifying an antigen-reactive cell that recognizes an endogenous antigen in complex with an MHC molecule expressed by a subject, the method comprising: (a) providing an antigen-presenting cell (APC) expressing an endogenous antigen in complex with an exogenous MHC molecule, wherein the exogenous MHC molecule is the MHC molecule expressed by the subject or derived from the subject; (b) contacting the APC with a plurality of TCR-expressing cells derived from the subject, wherein the plurality of TCR-expressing cells or a subset of the plurality of TCR-expressing cells recognizes the endogenous antigen in complex with the exogenous MHC of the APC, and wherein the plurality of TCR-expressing cells or a subset of the plurality of TCR-expressing cells that recognizes the endogenous antigen (i) is attached to a label secreted from the APC or a label transferred by a label-transferring enzyme associated with the APC upon recognizing the endogenous antigen, or (ii) expresses an activation marker upon recognizing the endogenous antigen; and (c) identifying the subset of the plurality of TCR-expressing cells based on the label or the activation marker, thereby identifying the antigen-reactive cell.

In some embodiments, identifying comprises enriching the subset of the plurality of TCR-expressing cells. In some embodiments, the APC expresses at least about 100 endogenous antigens. In some embodiments, the method further comprises determining whether to administer a cancer drug to the subject based on a fraction of the subset of the plurality of TCR-expressing cells in the plurality of TCR-expressing cells or the number of the TCR-expressing cells in the subset. In some embodiments, the method further comprises quantifying the number of the subset of the plurality of TCR-expressing cells. In some embodiments, the method further comprises quantifying the number of the plurality of TCR-expressing cells prior to contacting in (b). In some embodiments, the method further comprises determining a fraction of the subset of the plurality of TCR-expressing cells in the plurality of TCR-expressing cells. In some embodiments, the method further comprises determining whether to administer a cancer drug to the subject based on the fraction or the number of the TCR-expressing cells in the subset. In some embodiments, the method further comprises administering a cancer drug to the subject determined as being suitable for treatment with the cancer drug based on the fraction. In some embodiments, the method further comprises not administering a cancer drug to the subject determined as being unsuitable for treatment with the cancer drug based on the fraction. In some embodiments, the method further comprises increasing a dose of the cancer drug to the subject. In some embodiments, the method further comprises decreasing a dose of the cancer drug to the subject. In some embodiments, the cancer drug is an immune cell regulator. In some embodiments, the immune cell regulator is a cytokine or an immune checkpoint inhibitor.

In some embodiments, the method further comprises determining a TCR sequence of the subset of the plurality of TCR-expressing cells. In some embodiments, the method further comprises delivering a polynucleotide molecule having the TCR sequence into a recipient cell for expression. In some embodiments, the recipient cell does not comprise the TCR sequence prior to delivering. In some embodiments, an endogenous TCR of the recipient cell is inactivated. In some embodiments, the recipient cell is a T cell. In some embodiments, the T cell is an autologous T cell or an allogenic T cell. In some embodiments, the method further comprises administering the recipient cell or derivative thereof into the subject. In some embodiments, the subset of the plurality of TCR-expressing cells expresses at least two different TCRs. In some embodiments, the method further comprises determining sequences of the at least two different TCRs. In some embodiments, the method further comprises delivering a plurality of polynucleotide molecules encoding the at least two different TCRs into a plurality of recipient cells for expression. In some embodiments, the method further comprises contacting the plurality of recipient cells with the APC or an additional APC. In some embodiments, the method further comprises enriching a recipient cell from the plurality of recipient cells, which recipient cell recognizes the APC or the additional APC. In some embodiments, the label comprises a detectable moiety, which detectable moiety is detectable by flow cytometry. In some embodiments, the detectable moiety is a biotin, a fluorescent dye, a peptide, digoxigenin, or a conjugation handle. In some embodiments, the conjugation handle comprises an azide, an alkyne, a DBCO, a tetrazine, or a TCO. In some embodiments, the label comprises a substrate recognized by the label-transferring enzyme. In some embodiments, the label is a cytokine secreted by the APC. In some embodiments, the label-transferring enzyme is a transpeptidase or a glycosyltransferase. In some embodiments, the transpeptidase is a sortase. In some embodiments, the glycosyltransferase is a fucosyltransferase. In some embodiments, the label-transferring enzyme is expressed by the APC or is supplied outside and attached to the APC. In some embodiments, the label-transferring enzyme is a transmembrane protein. In some embodiments, the label-transferring enzyme is attached to the APC via covalent or non-covalent interaction. In some embodiments, the APC is derived from a subject. In some embodiments, the APC is a cancer cell line. In some embodiments, the subject has cancer. In some embodiments, the cancer cell line is derived from a same cancer type as the cancer of the subject. In some embodiments, the plurality of TCR-expressing cells comprises T cells. In some embodiments, the T cells are tumor-infiltrating T cells or peripheral T cells. In some embodiments, the T cells express LAG3, CD39, CD69, CD103, CD25, PD-1, TIM-3, OX-40, 4-1BB, CD137, CD3, CD28, CD4, CD8, CD45RA, CD45RO, GITR, FoxP3, or any combinations thereof. In some embodiments, the plurality of TCR-expressing cells comprises a label-accepting moiety, which label-accepting moiety receives the label.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure”, “Fig.”, and “FIGURE” herein) of which:

FIG. 1 depicts an example of using MHC-personalized cell line described herein in personalized T cell therapy.

FIGS. 2A-2F depict experimental data showing that multiple exogenous MHC alleles can be co-expressed in a cell line and achieve sufficient expression level and sufficient ability to present intracellularly expressed antigens. FIG. 2A shows data of T cells after being co-cultured with K562 cells comprising one exogenous HLA and an mRNA of a tandem minigene (TMG) encoding several epitopes including an HLA-A*02:01-restricted NY-ESO-1 epitope.

FIG. 2B shows data of T cells after being co-cultured with K562 cells comprising three exogenous HLAs and an mRNA of a TMG encoding several epitopes including an HLA-A*02:01-restricted NY-ESO-1 epitope. FIG. 2C shows data of T cells after being co-cultured with K562 cells comprising six exogenous HLAs and an mRNA of a TMG encoding several epitopes including an HLA-A*02:01-restricted NY-ESO-1 epitope. FIG. 2D shows data of T cells after being co-cultured with K562 cells comprising one exogenous HLA and an mRNA encoding an irrelevant epitope. FIG. 2E shows data of T cells after being co-cultured with K562 cells comprising three exogenous HLA and an mRNA encoding an irrelevant epitope.

FIG. 2F shows data of T cells after being co-cultured with K562 cells comprising six exogenous HLA and an mRNA encoding an irrelevant epitope.

FIGS. 3A-3F depict experimental data showing that B2M-MHC-I-alpha fusion can be abundantly expressed and transported to cell surface in MHC-null cells. FIG. 3A shows data detecting surface expression of MHC-I-alpha in K562 cells without exogenous HLA. FIG. 3B shows data detecting surface expression of MHC-I-alpha in K562 cells comprising an mRNA encoding an exogenous MHC allele, HLA-A*02:01. FIG. 3C shows data detecting surface expression of MHC-I-alpha in K562 cells without exogenous HLA and with B2M knocked out (K562-B2M^{KO}). FIG. 3D shows data detecting surface expression of MHC-I-alpha in K562-B2M^{KO} cells expressing an exogenous HLA-A*02:01. FIG. 3E shows data detecting surface expression of MHC-I-alpha in K562-B2M^{KO} cells expressing B2M-HLA-A*02:01 fusion. FIG. 3F shows data detecting surface expression of MHC-I-alpha in K562-B2M^{KO} cells expressing B2M-HLA-C*08:02 fusion.

FIGS. 4A-4K depict experimental data showing that B2M-MHC-I-alpha fusion can efficiently present intracellularly expressed antigens in MHC-null cells. T cells were analyzed by FACS after being co-cultured with three different MHC-engineered cell lines. FIG. 4A shows data for T cells after being co-cultured with K562/A*02:01 cells in the absence of exogenous antigen. FIG. 4B shows data for T cells after being co-cultured with K562-B2M^{KO}/A*02:01 cells in the absence of exogenous antigen. FIG. 4C shows data for T cells after being co-cultured with K562-B2M^{KO}/B2M-A*02:01 cells in the absence of exogenous antigen. FIG. 4D shows data for T cells after being co-cultured with K562/A*02:01 cells in the presence of antigen. FIG. 4E shows data for T cells after being co-cultured with K562-B2M^{KO}/A*02:01 cells in the presence of antigen. FIG. 4F shows data for T cells after being co-cultured with K562-B2M^{KO}/B2M-A*02:01 cells in the presence of antigen. FIG. 4G shows data for T cells after being co-cultured with K562/A*02:01 cells expressing the antigen from a TMG. FIG. 4H shows data for T cells after being co-cultured with K562-B2M^{KO}/A*02:01 cells expressing the antigen from a TMG. FIG. 4I shows data for T cells after being co-cultured with K562-B2M^{KO}/B2M-A*02:01 cells expressing the antigen from a TMG. FIG. 4J shows data for T cells without co-culture. FIG. 4K shows data for T cells after being co-cultured with K562-B2M^{KO}/Ag-B2M-A*02:01 cells.

FIGS. 5A-5F depict experimental data showing that B2M-MHC-I-alpha fusion can efficiently present endogenous antigens in cancer cells. FIG. 5A shows data of T cells after being co-cultured with PANC1 cell line without expressing any exogenous MHC. FIG. 5B shows data of T cells after being co-cultured with PANC1 cell line expressing an exogenous C*08:02. FIG. 5C shows data of T cells after being co-cultured with PANC1 cell line expressing an exogenous B2M-C*08:02 fusion. FIG. 5D shows data of T cells after being co-cultured with AsPC1 cell line without expressing any exogenous MHC. FIG. 5E shows data of T cells after being co-cultured with AsPC1 cell line expressing an exogenous C*08:02. FIG. 5F shows data of T cells after being co-cultured with AsPC1 cell line expressing an exogenous B2M-C*08:02 fusion.

FIG. 6A depicts experimental data showing kinetics of surface expression of exogenous HLA alleles in MALME3M cancer cell line.

FIG. 6B depicts experimental data showing kinetics of surface expression of exogenous HLA alleles in HMBC cancer cell line.

FIG. 7 depicts an example workflow of TCR identification using synthetic library and cancer cell line.

FIG. 8 depicts detection of HLA-A02:01 in HLA-A02:01 positive (HLA-A02:01+) cancer cell lines.

FIG. 9 depicts flow cytometry plots from four different co-cultures of engineered T cells co-cultured with HLA-A02:01 negative or positive cancer cell lines, where the cells displayed are live synthetic TCR-T cells stained with “pre” and “post” CD137.

FIG. 10A depicts a volcano plots of FACS data showing that the model TCRs along with other unknown TCRs were enriched in the positive control co-culture with HMCB-TMG but were not enriched in the HLA-A02:01 negative cell line SKMEL.

FIG. 10B depicts a volcano plots of MACS data showing that the model TCRs along with other unknown TCRs were enriched in the positive control co-culture with HMCB-TMG but were not enriched in the HLA-A02:01 negative cell line SKMEL

FIG. 11A depicts bar graphs showing expression of identified TCRs in cells. The highest recovery of CD3 was observed 48 hrs post electroporation (EP), indicating TCR expression.

FIG. 11B depicts double-knockout cells (with endogenous TRAC and TRBC knocked out) expressing the identified TCRs were co-cultured with HLA-A02:01 positive or negative cancer cell line and the percentage of the activated population of cells were determined by CD137 upregulation.

FIG. 12 depicts experimental data showing results of a killing assay using the identified TCRs co-cultured with APCs, where the APCs are HLA-A02:01 positive expressing a tandem mini gene (TMG) containing known antigens (MUT) or other antigens (WT).

FIG. 13A depicts experimental data showing the upregulation of an early activation marker CD137 only in response to the parental cell line expressing the patient's restricting HLA.

FIG. 13B depicts experimental data of cell lysis as monitored by an lactate dehydrogenase (LDH) assay.

FIG. 13C depicts experimental data of a co-culture assay, where apoptosis was monitored by a Caspase-Glo® 3/7 assay.

FIG. 13D depicts experimental data of a co-culture assay, where cytokine release from activated T cells was measured.

FIG. 14 depicts a volcano plot for individual TCR sequences as a function of fold enrichment (compared to pre-selection frequencies) and P value.

### DETAILED DESCRIPTION OF THE INVENTION

In this disclosure, the use of the singular includes the plural unless specifically stated otherwise. Also, the use of “or” means “and/or” unless stated otherwise. Similarly, “comprise,” “comprises,” “comprising” “include,” “includes,” and “including” are not intended to be limiting.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “enriching,” “isolating,” “separating,” “sorting,” “purifying,” “selecting” or equivalents thereof can be used interchangeably and refer to obtaining a subsample with a given property from a sample. For example, enriching can comprise obtaining a cell population or cell sample that contains at least about 2%, 5%, 10%, 20%, 30%, 40%, 50%, 55%, 60%, 65%, 70%, 75%, 80%, 85%, 90%, 95%, or 100% of the desired cell lineage or a desired cell having a certain cell phenotype, e.g., expressing a certain cell marker or not expressing a certain cell marker gene characteristic of that cell phenotype.

The term “cancer cell line,” as used herein, refers to an immortalized cell line derived from a cancer or tumor cell. The cancer cell line can comprise immortal cells that continually divide and grow over time under laboratory conditions. The immortalized cell line can be cultured for at least about 10, 20, 30, 40, 50, or more generations.

The term “subject,” as used herein, refers to an organism such as a mammal, which can be the object of a treatment, an observation or an experiment. The subject can be an individual, a host, or a patient (e.g., a cancer patient). Examples of subjects include, but are not limited to, horses, cows, camels, sheep, pigs, goats, dogs, cats, rabbits, guinea pigs, rats, mice (e.g., humanized mice), gerbils, non-human primates (e.g., macaques), humans and the like, non-mammals, including, e.g., non-mammalian vertebrates, such as birds (e.g., chickens or ducks), fish (e.g., sharks) or frogs, and non-mammalian invertebrates, as well as transgenic species thereof. In some cases, a subject can be a single organism (e.g., human). The subject can be a human having a tumor. In some cases, a subject can be a group of individuals comprising a small cohort having either a common immune factor to study and/or a disease, and/or a cohort of individuals without the disease (e.g., negative/normal control). A subject from whom samples are obtained can have a condition (e.g., a disease, a disorder, an allergy, an infection, cancer or autoimmune disorder or the like) and can be compared against a negative control subject who does not have the condition.

The term “derived” used in the context of a molecule or a cell refers to a molecule or a cell obtained or originated from a subject or a sample. A molecule derived from a subject or a sample can be a molecule isolated from the subject or the sample. A molecule derived from a subject or a sample can be a copy or a variant of a reference molecule contained (e.g., expressed) in or obtained from the subject or the sample. For example, a polypeptide molecule or a polynucleotide molecule derived from a subject or a sample can be a copy (e.g., an amplified copy, a chemically or enzymatically synthesized copy) of a reference molecule expressed in the subject or the sample. The polypeptide molecule or the polynucleotide molecule may have a sequence having at least about 30%, 35%, 40%, 45%, 50%, 55%, 60%, 65%, 70%, 75%, 80%, 85%, 90%, 95%, or 100% sequence identity with the reference molecule from the subject or the sample. A cell derived from a subject or a sample can be a cell isolated from the subject or the sample. A cell derived from a subject or a sample can be a copy or a variant of a reference cell contained in or obtained from the subject or the sample. For example, a cell derived from a subject or a sample can be an offspring cell of the reference cell from the subject or the sample during expansion or division. The cell derived from the subject or the sample may have been engineered or manipulated such that it may have a genetic profile (e.g., genomic or transcriptomic profile) or phenotypic profile different from the reference cell from the subject or the sample.

The term “exogenous,” as used herein, refers to a substance present in cells or organisms other than its own native source. For example, a cancer cell line may express HLA-A*02:01 and/or HLA*11:01 but does not express HLA*A24:02. If a nucleic acid sequence encoding the HLA*A24:02 is introduced to the cancer cell line, the HLA*A24:02 or the nucleic acid sequence encoding it can be referred to as exogenous to the cancer cell line. On the other hand, the term “endogenous” refers to a substance that is native to the cells or organisms. In this example, HLA-A*02:01 and/or HLA*11:01 can be referred to as endogenous to the cancer cell line.

The term “exogenously expressing” or “exogenously expressed” refers to an expression of a polypeptide from an exogenous polynucleotide sequence (e.g., a polynucleotide sequence not derived or originated from the host cell) introduced to the host cell. An exogenous protein can be a protein expressed by an exogenous polynucleotide sequence that is not derived or originated from the host cell.

The term “cognate pair,” as used herein, refers to an original or native pair of two nucleic acid molecules or proteins encoded by the two nucleic acid molecules that are contained within or derived from an individual cell. The cognate pair can be natively paired chains within the individual cell. For example, a cognate pair of T-cell receptor (TCR) can be a natively paired TCR alpha and beta chains within or derived from an individual cell. For another example, a cognate pair of T-cell receptor (TCR) can be a natively paired TCR gamma and delta chains within or derived from an individual cell.

The term “tumor-experienced,” as used herein, refers to being contacted with or exposed to a tumor cell or derivative thereof, an offspring of a tumor cell, or a tumor antigen. In some cases, a tumor-experienced T cell may have been exposed to a tumor cell or a tumor antigen. In some cases, a tumor-experienced T cell may be a PD-thigh cell.

### Overview

Tumor-associated antigens (TAAs) and tumor-specific antigens (TSAs) can be expressed in not only primary tumors but also cancer cell lines of the same, or even different organ/tissue origin. For example, some TAAs expressed in the tumor of a given lung patient may also be expressed in some lung cancer cell lines such as NCI-H1734, HCC2935, NCI-H3255, HCC4006, and RERFLCAD1 among many others. Although antigen-reactive T cells may be screened by using primary tumor sample, most of the time, the primary tumor sample cannot be reliably obtained in sufficient quality and/or quantity. The compositions and methods provides herein use cancer cell line to identify antigen-reactive (e.g., tumor-reactive) T-cell receptors (TCRs), which overcome the limitations with using primary tumor sample for screening antigen-reactive T cells. The compositions or methods provided herein can be non-invasive since a tumor sample may not need to be obtained from a patient. The compositions or methods provided herein can be used to formulate personalized immunotherapies for subjects having a disease such as cancer.

The cancer cell line, however, may express a different set of WIC molecules than the patient. In some cases, patient's autologous dendritic cells (DCs, such as monocyte-derived DCs, or MoDCs, MDDCs) can be fed with cancer cell line, since the autologous DCs express the patient's WIC molecules. The autologous DCs fed with cancer cell lines may be used as an alternative to autologous DCs fed with autologous cancer cells. The cancer cell line or autologous cancer cells can be killed or lysed first (e.g., by irradiation, freeze-thaw cycle and/or chemicals such as mitomycin C and hypochlorous acid). However, autologous DCs in sufficient quantity may not be available, and the TAA/TSAs expressed in the cancer cell line may not be sufficiently presented by the autologous DCs under certainly conditions. In some other cases, the cancer cell line can be engineered (or personalized or WIC-personalized) by exogenously express the patient's MHC(s) in the cancer cell line. Optionally, the expression of the cancer cell line's endogenous MHC(s) can be abolished to reduce the chance of T cell or TCR activation due to alloreactivity.

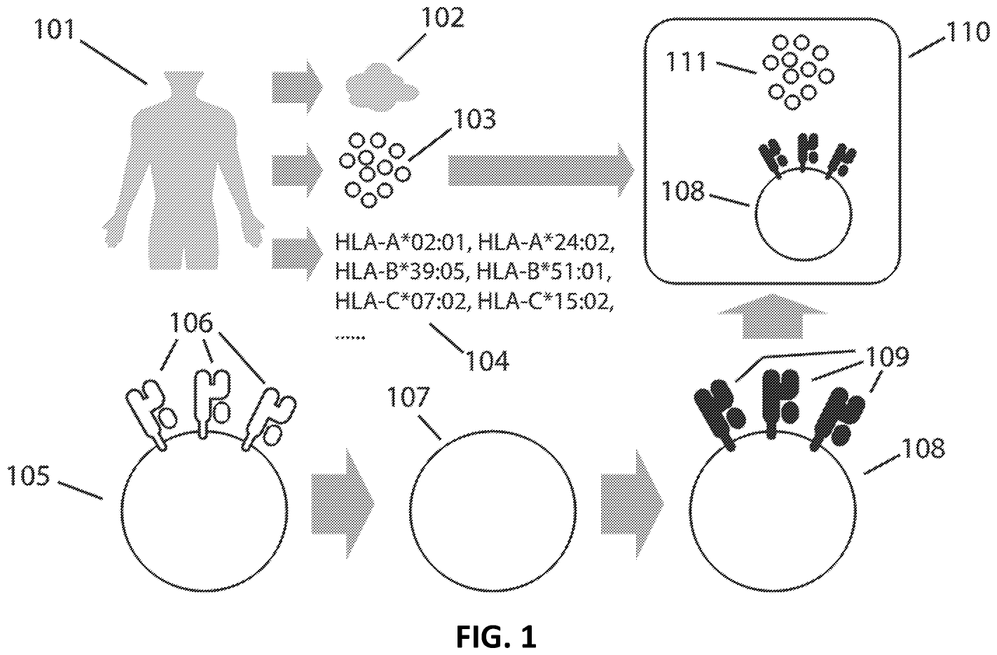

FIG. 1 shows an example of using WIC-personalized cell line described herein in personalized T cell therapy. One or more information can be obtained from a subject 101 (e.g., a cancer patient in need of a treatment). A tumor sample 102 may be obtained from a subject 101. T cells 103 may also be obtained from the subject 101. HLA alleles 104 carried by the subject 101 may also be determined. A cancer cell line 105 can be chosen or identified which may have similar gene expression profile as the tumor sample 102. The endogenous MHC molecules 106 of the cancer cell line 105 may be inactivated to make the MHC-null version of the cancer cell line 107. The WIC-null cancer cell line 107 can be engineered to express exogenous MHC molecules 109 to generate the MHC-engineered cancer cell line 108. The genes encoding these exogenous WIC molecules can be chosen based on HLA alleles 104 and delivered to the cancer cell line 107 using methods described herein. TCR-expressing cells 111 can be mixed (e.g., cocultured in 110) with the MHC-engineered cancer cell line 108 for TCR identification. The TCRs in the TCR-expressing cells 111 may overlap with or be derived from the TCRs in the T cells 103 from the subject 101.

An example method provided herein for identifying an antigen-reactive cell that recognizes an endogenous antigen of a cancer cell line in complex with an MHC molecule expressed by a subject can comprise: (a) providing a cell that is a cancer cell line expressing an endogenous antigen in complex with an exogenous WIC molecule, wherein the exogenous MHC molecule is the WIC molecule expressed by the subject or derived from the subject; (b) contacting the cancer cell line with a first plurality of TCR-expressing cells, wherein the first plurality of TCR-expressing cells or a subset of the first plurality of TCR-expressing cells is activated by the endogenous antigen in complex with the exogenous MHC of the cancer cell line; and (c) subsequent to contacting in (b), enriching the subset of the first plurality of TCR-expressing cells, thereby identifying the antigen-reactive cell that recognizes the endogenous antigen of the cancer cell line. Related compositions are also provided herein.

The compositions and methods can also be used for evaluating an anti-cancer activity of a TCR-expressing cell. For example, a method for evaluating an anti-cancer activity of a TCR-expressing cell can comprise: (a) providing a plurality of cells, wherein the plurality of cells is derived from a cancer cell line and express an endogenous antigen in complex with an exogenous MHC molecule, wherein the exogenous WIC molecule is an MHC molecule expressed by a subject or derived from the subject; (b) contacting the plurality of cells with a plurality of TCR-expressing cells expressing a plurality of TCRs derived from the same subject, wherein the plurality of TCRs or a fraction thereof recognizes the endogenous antigen in complex with the exogenous MHC molecule of the plurality of cells or a fraction thereof; and (c) subsequent to contacting in (b), quantifying (i) the fraction of the plurality of cells that are recognized by the plurality of TCR-expressing cells or a fraction thereof, (ii) the fraction of the plurality of TCR-expressing cells that recognize the plurality of cells or a fraction thereof, and/or (iii) a cytokine secreted by the plurality of TCR-expressing cells or a fraction thereof.

### T-Cell Receptor (TCR)

The TCR can be used to confer the ability of T cells to recognize antigens (e.g., T cell epitopes) associated with various cancers or infectious organisms. The TCR can be made up of both an alpha (α) chain and a beta (β) chain or a gamma (γ) and a delta (δ) chain. The proteins which make up these chains can be encoded by DNA, which employs a unique mechanism for generating the tremendous diversity of the TCR. This multi-subunit immune recognition receptor can associate with the CD3 complex and bind peptides presented by the MHC class I and II proteins on the surface of antigen-presenting cells (APCs). Binding of a TCR to the antigenic peptide on the APC can be a central event in T-cell activation, which occurs at an immunological synapse at the point of contact between the T cell and the APC.

The TCR may recognize the T cell epitope in the context of an major histocompatibility complex (MHC) class I molecule. MHC class I proteins can be expressed in all nucleated cells of higher vertebrates. The MHC class I molecule is a heterodimer composed of a 46-kDa heavy chain which is non-covalently associated with the 12-kDa light chain beta-2-microglobulin (or β-2-microglobulin or B2M). The human MHC is also called the human leukocyte antigen (HLA) complex. In humans, there are several MHC alleles, such as, for example, HLA-A2, HLA-A1, HLA-A3, HLA-A24, HLA-A28, HLA-A31, HLA-A33, HLA-A34, HLA-B7, HLA-B45 and HLA-Cw8. In some embodiments, the MHC class I allele is an HLA-A2 allele, which in some populations is expressed by approximately 50% of the population. In some embodiments, the HLA-A2 allele can be an HLA-A*0201, *0202, *0203, *0206, or *0207 gene product. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206 and 23% HLA-A*0203.

In some embodiments, the TCR may recognize the T cell epitope in the context of an MHC class II molecule. MHC class II proteins can be expressed in a subset of APCs. In humans, there are several MHC class II alleles, such as, for example, DR1, DR3, DR4, DR7, DR52, DQ1, DQ2, DQ4, DQ8 and DPI. In some embodiments, the MHC class II allele is an HLA-DRB1*0101, an HLA-DRB*0301, an HLA-DRB*0701, an HLA-DRB*0401 or an HLA-DQB1*0201 gene product.

The TCR chains can comprise a variable domain (or variable region) and a constant domain (or constant region). A full-length constant domain/region can comprise an extracellular portion (referred to as “extracellular constant domain” herein), a hinge region, a transmembrane region, and a cytoplasmic tail. In various embodiments, a constant domain can be a full-length constant domain or a portion thereof, for example, the extracellular constant domain. The variable domain of TCRα and δ chains is encoded by a number of variable (V) and joining (J) genes, while TCRβ and γ chains are additionally encoded by diversity (D) genes. During VDJ recombination, one random allele of each gene segment is recombined with the others to form a functional variable domain. Recombination of the variable domain with a constant gene segment can result in a functional TCR chain transcript. Additionally, random nucleotides may be added and/or deleted at the junction sites between the gene segments. This process can lead to strong combinatorial (depending on which gene regions will recombine) and junctional diversity (depending on which and how many nucleotides will be added/deleted), resulting in a large and highly variable TCR repertoire, which can ensure the identification of a plethora of antigens. Additional diversity can be achieved by the pairing (also referred to as “assembly”) of α and β or γ and δ chains to form a functional TCR. By recombination, random insertion, deletion and substitution, the small set of genes that encode the T cell receptor has the potential to create between 10¹⁵ and 10²⁰ TCR clonotypes. As used herein, a “clonotype” refers to a population of immune cells that carry an identical immunoreceptor. For example, a clonotype refers to a population of T cells that carry an identical TCR, or a population of B-cells that carry an identical BCR (or antibody). “Diversity” in the context of immunoreceptor diversity refers to the number of immunoreceptor (e.g., TCR, BCR and antibody) clonotypes in a population. The higher diversity in clonotype may indicate higher diversity in cognate pair (e.g., native pair) combination.

Each TCR chain can contain three hypervariable loops in its structure, termed complementarity determining regions (CDR1-3). CDR1 and 2 are encoded by V genes and may be functional for interaction of the TCR with the MHC complex. CDR3, however, is encoded by the junctional region between the V and J or D and J genes and therefore can be highly variable. CDR3 may be the region of the TCR in direct contact with the peptide antigen. CDR3 can be used as the region of interest to determine T cell clonotypes. The sum of all TCRs by the T cells of an individual or a sample is termed the TCR repertoire or TCR profile. The TCR repertoire can change with the onset and progression of diseases. Therefore, determining the immune repertoire status under different disease conditions, such as cancer, autoimmune, inflammatory and infectious diseases may be useful for disease diagnosis and prognosis.

The TCR may be a full-length TCR as well as antigen-binding portion or antigen-binding fragment (also called MHC-peptide binding fragment) thereof. In some embodiments, the TCR is an intact or full-length TCR. In some embodiments, the TCR is an antigen-binding portion that is less than a full-length TCR but that binds to a specific antigenic peptide bound to an MHC molecule, e.g., an MHC-peptide complex. An antigen-binding portion or fragment of a TCR can contain only a portion of the structural domains of a full-length or intact TCR, but yet is able to bind the epitope (e.g., MHC-peptide complex) to which the full TCR binds. In some cases, an antigen-binding portion or fragment of a TCR contains the variable domains of a TCR, such as variable a chain and variable R chain of a TCR, sufficient to form a binding site for binding to a specific MHC-peptide complex, such as generally where each chain contains three complementarity determining regions. Polypeptides or proteins having a binding domain which is an antigen-binding domain or is homologous to an antigen-binding domain are included.

### Methods for TCR Identification

The present disclosure provides compositions and methods to identify antigen-reactive cells or TCRs (e.g., subject-derived TCRs) that are reactive to an antigen of interest, thereby allowing for the discovery of therapeutically relevant antigen-reactive cells or TCRs. The identified antigen-reactive cells or TCRs can be tumor reactive or can recognize tumor antigens. The present disclosure also provides methods to evaluate or analyze anti-cancer activity of a TCR-expressing cell. In various embodiments, the cancer cell line or the TCR-expressing cell described herein comprises (e.g., expresses) subject-specific MHC molecules or TCRs, allowing for the formulation of personalized cell-based immunotherapy.

The compositions and methods provided herein can be used to identify an antigen-reactive cell or a TCR of the antigen-reactive cell that recognizes an endogenous antigen of a cancer cell line in complex with an MHC molecule expressed by a subject (e.g., a human patient). For example, in some aspects, the method can comprise providing a cell that is a cancer cell line expressing an endogenous antigen in complex with an exogenous MHC molecule. The exogenous MHC molecule is the MHC molecule expressed by the subject or derived from the subject. Next, the cancer cell line can be contacted (e.g., cocultured) with a first plurality of TCR-expressing cells. In some cases, the cancer cell line can be contacted with a mixture comprising the first plurality of TCR-expressing cells. Upon contacting with the cancer cell line, the first plurality of TCR-expressing cells or a subset of the first plurality of TCR-expressing cells can be activated by the endogenous antigen in complex with the exogenous MHC of the cancer cell line. Subsequent to contacting, the subset of the first plurality of TCR-expressing cells can be identified. For example, the subset of the first plurality of TCR-expressing cells can be enriched or selected from the first plurality of TCR-expressing cells. The antigen-reactive cell or the TCR of the antigen-reactive cell that recognizes the endogenous antigen of the cancer cell line can be identified from the enriched or selected subset. In some cases, identifying described herein can comprise enriching the subset that can be activated by the endogenous antigen in complex with the exogenous MHC of the cancer cell line. In some cases, identifying can comprise selecting the subset or separating the subset from those that do not recognize the endogenous antigen in complex with the exogenous MHC of the cancer cell line. As described herein, enriching can comprise expanding the subset by coculturing the subset with APCs (including artificial APCs) or isolating the subset by flow cytometry-based methods such as FACS or MACS. In some cases, selecting can comprise separating the subset by flow cytometry-based methods.

The exogenous MHC molecule can be exogenous to the cancer cell line. The exogenous MHC molecule can be derived from the subject. Various methods can be used to obtain the information of which MHC allele or alleles a subject expresses. For example, a peripheral blood sample can be obtained from the subject and genomic DNA can be extracted. The MHC gene loci can be amplified and sequenced. The sequences obtained from sequencing can be compared to reference MHC sequences from various databases. Alternatively, the MHC allele or alleges expressed by a subject can be determined by polymerase chain reaction or antibody-based methods.

Optionally, the method can further comprise providing a non-cancer cell expressing an additional endogenous antigen in complex with an exogenous MHC molecule. The exogenous MHC molecule can be derived from the same subject. The non-cancer cell can exogenously express at least one, two, three, four, five, six, seven, eight, nine, ten or more different MHC molecules identified in a subject. The non-cancer cell can be used as a negative control to select antigen-reactive cells that are self-reactive (e.g., cells that recognize self-antigens or autoantigens) and may not be used to formulate immunotherapies to treat a patient. Next, the non-cancer cell can be contacted with a second plurality of TCR-expressing cells. The second plurality or a subset of the second plurality of TCR-expressing cells can be activated by the additional endogenous antigen in complex with the exogenous MHC of the non-cancer cell. The additional endogenous antigen can be the same as or different from the endogenous antigen expressed by the cancer cell line. The non-cancer cell may not express the endogenous antigen expressed by the cancer cell line. The non-cancer cell may express the endogenous antigen expressed by the cancer cell line at a lower level. The non-cancer cell may express the endogenous antigen expressed by the cancer cell line, but may not present the endogenous antigen expressed by the cancer cell line.

Optionally, in some cases, a negative selection can be carried out using a cancer cell line which does not express endogenous MHC molecules such as MHC-null cancer cell line described herein. These MHC-null cancer cell line can be used to select TCR-expressing cells that recognize non-MHC restricted antigens on the surface of the cancer cell line. These selected TCR-expressing cells may not recognize the endogenous antigens of the cancer cell line that are also tumor antigens.

The negative selection may be optional. If the negative selection is carried out, the first plurality and the second plurality can be aliquots from a same sample. The first plurality and the second plurality of TCR-expressing cells can be derived from a same sample. The first plurality and the second plurality of TCR-expressing cells can express a same TCR. The first plurality and the second plurality of TCR-expressing cells can comprise a same population of TCRs (e.g., a population of at least about 5, 10, 20, 50, 100, 200, 500, 1,000, 10,000, 100,000, 1,000,000 or more different TCRs). The first plurality or the second plurality of TCR-expressing cells may express different TCRs. The TCRs can be derived from a subject, and these TCRs can be subject-specific TCRs. The method can further comprise identifying (e.g., enriching or selecting) the subset of the second plurality of TCR-expressing cells.

The identifying described herein can comprise selecting the subset of the first plurality of TCR-expressing cells and/or the subset of the second plurality of TCR-expressing cells based on a marker. For example, selecting the subset of the first plurality of TCR-expressing cells and/or the subset of the second plurality of TCR-expressing cells can comprise using FACS or MACS based on the marker. In some cases, the selection may be based on binding to soluble, fluorescently labeled, or surface-bound peptide MHC complex (pMHC), pMHC tetramer or pMHC oligomer. The selection may be based on marker expression on the TCR-expressing cells after the cells contact MHC-bound antigen. The marker may be a cell surface marker. The cell surface marker may be CD39, CD69, CD103, CD25, PD-1, TIM-3, OX-40, 4-1BB, CD137, CD3, CD28, CD4, CD8, CD45RA, CD45RO, GITR, FoxP3, as well as other T cell activation markers, or a combination thereof. The selection may be based on calcium influx. The marker may be intracellular protein or a secreted protein. The intracellular protein may be a transcription factor or may be a phosphorylated protein. The secreted protein may be a cytokine or a chemokine (e.g., IFN-γ, TNF-alpha, IL-17A, IL-2, IL-3, IL-4, GM-CSF, IL-10, IL-13, granzyme B, perform, or a combination thereof). When using a secreted protein as the marker, inhibitors of protein trafficking may be added to the cell. The inhibitor of protein trafficking may be a Golgi blocker. The Golgi blocker may be Brefeldin A, Monensin or the like. The secreted protein may be IL-2, IL-10, IL-15, TNF-alpha, or INF-gamma. The selection may also be based on reporter gene expression or a reporter protein. The reporter protein may be a fluorescent protein (such as GFP and mCherry). The reporter gene expression may be under the control of a transcription factor which is regulated by TCR signaling. Examples of these transcription factors include, but are not limited to, AP-1, NFAT, NF-kappa-B, Runx1, Runx3, etc.

The method can further comprise identifying a TCR that is expressed in the subset of the first plurality of TCR-expressing cells, but not in the subset of the second plurality of TCR-expressing cells. The method can further comprise identifying a TCR of a cell in the subset of the first plurality of TCR-expressing cells that is activated by the endogenous antigen in complex with the exogenous MHC of the cancer cell line, and that is in a cell in the second plurality of TCR-expressing cells that is not activated by the additional endogenous antigen in complex with the exogenous MHC of the non-cancer cell. Various sequencing methods can be used to identify the TCR that is expressed in the subset of the first plurality of TCR-expressing cells, but not in the subset of the second plurality of TCR-expressing cells.

The non-cancer cell can be a stem cell, a normal cell, or a primary healthy cell. The non-cancer cell can be a mammalian cell such as a human cell. The non-cancer cell can be obtained from a healthy subject or a non-cancer sample from a patient. The non-cancer cell can be immortalized. For example, the non-cancer cell can be an immortalized primary cell by overexpressing SV40. The stem cell can be an induced pluripotent stem cell (iPSC). The non-cancer cell can be an differentiated iPSC. The non-cancer cell can express an autoimmune regulator (AIRE).

The endogenous MHC molecule (e.g., gene or protein product) of the cancer cell line or the non-cancer cell can be inactivated (e.g., down regulated, knocked down, or knocked out). The endogenous MHC molecule that are inactivated may not be expressed on the cell surface. A gene encoding a MHC molecule or a subunit thereof can be inactivated. A gene regulating the expression of the gene encoding a MHC molecule or a subunit thereof can be inactivated. The protein product of the gene encoding a MHC molecule or a subunit thereof can be inactivated, for example, by degradation or inhibition. The protein product of the gene regulating the expression of the gene encoding a MHC molecule or a subunit thereof can be inactivated. The endogenous MHC molecule, when being inactivated, may have an expression level at most about 60%, 50%, 40%, 30%, 20%, 10%, 5%, 1%, 0.1% or less than normal expression level in the cancer cell line. The endogenous MHC molecule may be completely inactivated such that no expression can be detected using various methods. The cancer cell line with endogenous MHC molecule inactivated can be an MHC-null cancer cell line. The non-cancer cell can be an MHC-null non-cancer cell. The cancer cell line or non-cancer cell can be null for an endogenous MHC molecule. The cancer cell line or non-cancer cell can be null for at least 1, 2, 3, 4, 5, 6, 7, 8, 9, or more endogenous MHC molecules. In some cases, the cancer cell line or non-cancer cell can be null for all endogenous MHC molecules (including all class I or class II MHC molecules). The endogenous MHC molecule can comprise a MHC class I molecule, a MHC class II molecule, or a combination thereof. The MHC class I molecule can comprise HLA-A, HLA-B, HLA-C, or any combination thereof.

Various gene editing methods can be used to inactivate a gene encoding a MHC molecule or a subunit thereof, or inactivate a gene regulating the expression of the gene encoding a MHC molecule or a subunit thereof. In some cases, an alpha chain of the MHC class I molecule (MHC-I alpha) can be inactivated. In some cases, a gene encoding the alpha chain of the MHC class I molecule can be inactivated. In some cases, a beta-2-microglobulin (B2M) of the MHC class I molecule can be inactivated. In some cases, a gene encoding the B2M of the MHC class I molecule is inactivated. In some cases, one or more genes encoding MHC molecules can be inactivated. For example, both gene encoding B2M and gene encoding alpha chain of MHC class I molecule can be inactivated. The MHC class II molecule can comprise HLA-DP, HLA-DM, HLA-DOA, HLA-DOB, HLA-DQ, HLA-DR, or any combination thereof. In some cases, an alpha chain or a beta chain of the MHC class II molecule can be inactivated. In some cases, a gene encoding the alpha chain or the beta chain of the MHC class II molecule can be inactivated. In some cases, a gene regulating transcription of the MHC class II molecule can be inactivated. For example, the gene CIITA can be inactivated. In some cases, both genes encoding MHC class II molecules and genes regulating transcription of the MHC class II molecules can be inactivated.

The exogenous MHC molecule of the cancer cell line or the non-cancer cell can comprise a MHC class I molecule, a MHC class II molecule, or a combination thereof, derived from the subject (e.g., the same subject from which the TCRs are obtained). The MHC class I molecule can comprise HLA-A, HLA-B, HLA-C, or any combination thereof. The MHC class II molecule can comprise HLA-DP, HLA-DM, HLA-DOA, HLA-DOB, HLA-DQ, HLA-DR, or any combination thereof. The exogenous MHC molecule can comprise an MHC-I alpha derived from the subject and an endogenous B2M. The exogenous MHC molecule can comprise both an MHC-I alpha and a B2M derived from the subject. The exogenous MHC molecule can be a fusion protein of the MHC-I alpha and the B2M (B2M-MHC-I-alpha fusion). The MHC-I alpha and the B2M can be linked by a linker. The linker can be (G₄S)n, wherein G is glycine, S is serine, and n is an integer from 1 to 10. The exogenous MHC molecule can comprise an MHC-II alpha and an MHC-II beta derived from the subject.

The plurality of TCR-expressing cells can be isolated from the same subject. The plurality of TCR-expressing cells can comprise a primary T cell. The primary T cell can be a tumor-infiltrating T cell. The primary T cell can be a peripheral T cell. The peripheral T cell can be a tumor-experienced T cell, which may have been contacted with the cancer cells or offspring of the cancer cells, or may have been exposed to tumor antigens. The tumor-experienced T cell may be PD-1+ T cell. The tumor-experienced T cell may have a high PD-1 expression. For example, when measuring PD-1 expression on the T cell surface, cells having PD-1 expression level of the top at least about 0.1%, 1%, 2%, 3%, 4%, 5%, 6%, 7%, 8%, 9%, 10% or more can be regarded as the T cell having a high PD-1 expression. The peripheral T cell may be a PD-1+ T cell. The primary T cell can be a CD4+ T cell, a CD8+ T cell, or a combination thereof. The primary T cell can be a cytotoxic T cell, a memory T cell, a regulatory T cell, a national killer T cell, an alpha beta T cell, a gamma delta T cell, or any combination thereof.

The plurality of TCR-expressing cells can comprise an engineered cell. The engineered cell can be various types of cells described herein. The engineered cell can express an exogenous TCR. The exogenous TCR can be derived from a primary T cell isolated from the same subject. The exogenous TCR can be subject-derived or subject-specific TCR.

The method can further comprise, prior to providing the cancer cell line, isolating a primary cancer cell or a tumor/cancer sample from the subject. The primary cancer cell can be obtained from various tissue samples described herein, for example, peripheral blood sample or a tumor tissue sample. The method can further comprise conducting transcriptomic (e.g., gene expression profile) or genomic analysis of the primary cancer cell or the tumor sample and some candidate cancer cell lines to identify the cancer cell line having a transcriptomic profile (e.g., gene expression profile) or genomic alteration (e.g., mutations) that resembles a primary cancer cell or a tumor sample isolated from the subject. The primary cancer cell and the cancer cell line can be from the same tissue origin. The gene expression profile, the transcriptomic profile or genomic alteration of the cancer cell line can be substantially similar to the primary cancer cell or the tumor sample. Transcriptomics (or gene expression profiling) can be used to measure the expression level of mRNAs (transcripts) in a cell population at a certain time. The gene expression profile between two samples can be compared by various methods. For example, the gene expression profile of each sample can be obtained by RNA-Seq or expression microarray. In some embodiments, RNA-Seq is used. In some cases, the RNA-Seq platforms used between the patient's cancer sample and cell lines, or among different cell lines, may be different. In these cases, tools such as ComBat can be used to correct for these sequencing platform differences or batch effects. The transcript counts can be summarized to the gene level and transcript per million (TPM) values can be obtained using standard methods. The data from different samples can be upper-quartile normalized and log-transformed.

A subset of genes can be used to calculate the Spearman's correlation between the patient's cancer sample and a cell line. The subset can be chosen based on whether the gene is correlated with purity of tumor sample, or based on variability of this gene in different tumor samples in the same cancer type or different cell lines. Public databases such as The Cancer Genome Atlas (TCGA) can be a useful resource for this and other purposes. For example, tumor purity estimates for all TCGA samples can be obtained using the ABSOLUTE46 method from the TCGA PanCan site or using ESTIMATE47. For a given cancer type, genes that have high correlations with tumor purity can be removed and the gene expression data can be adjusted for tumor purity using linear regression. Afterwards, the 5000 most variable genes ranked by interquartile range (IQR) across the primary tumor samples can be selected.

The gene expression profile of the patient's tumor sample can be purity-adjusted by comparing the gene expression profile of the patient's tumor and that of the TCGA data of the same cancer type using the methods described above. After this adjustment, the Spearman correlation between the patient's tumor sample and the cell line can be calculated using the normalized and log-transformed TPM values of the 5000 selected genes. The Spearman correlation coefficient may be used to describe the resemblance between the patient's tumor and a cell line. The resemblance may be considered sufficient if the correlation coefficient is equal to or greater than about 0, 0.1, 0.2, 0.3, 0.4, 0.5, 0.6, 0.7, 0.8 or more.

The method can further comprise identifying a TCR of the enriched subset of the plurality of TCR-expressing cells. The further comprising identifying a TCR (or the sequence of the TCR) expressed by the antigen-reactive cell. In some cases, identifying the TCR can comprise sequencing a TCR repertoire of the subset of the plurality of TCR-expressing cells. A frequency of each unique TCR of the subset can be determined in the sequencing data, which can be referred to as post-selection frequency. In some cases, a TCR repertoire of the plurality of TCR-expressing cells prior to contacting with the cancer cell line is subject to sequencing. A frequency of each unique TCR can be determined in the sequencing data, which can be referred to as pre-selection frequency. A TCR expressed by the antigen-reactive cell can be determined by comparing the post-selection frequency and the pre-selection frequency.

Various sequencing methods can be used herein. Various sequencing methods include, but are not limited to, Sanger sequencing, high-throughput sequencing, sequencing-by-synthesis, single-molecule sequencing, sequencing-by-ligation, RNA-Seq, Next generation sequencing (NGS), Digital Gene Expression, Clonal Single MicroArray, shotgun sequencing, Maxim-Gilbert sequencing, or massively-parallel sequencing. The TCR-expressing cells can be used as input for single-cell RNA-Seq methods such as inDrop or DropSeq. For example, the sequencing may use single cell barcoding (e.g., partitioning the TCR-expressing cells into individual compartment, barcoding nucleic acids released from a single cell, sequencing the nucleic acids, and pair the TCR chains from a single cell based on a same barcode). The sequencing may not comprise using a barcode if the sequence encoding the paired TCR chains within a cell has been fused or linked in a single continuous polynucleotide chain.

Optionally, the TCR or a sequence encoding the TCR identified herein can be introduced into a host cell (or a recipient cell) for expressing the TCR. The host cell can be administered into a subject in need thereof.

The method can further comprise administering (i) the antigen-reactive cell or (ii) a cell (e.g., a host cell) comprising the TCR of the antigen-reactive cell or (iii) a cell comprising a sequence encoding the TCR of the antigen-reactive cell into the subject. In some cases, the method can further comprise administering a therapeutically effective amount of the antigen-reactive cells or cells comprising the TCRs of the antigen-reactive cells into the subject. The antigen-reactive cell or a cell comprising the TCR of the antigen-reactive cell can be used to manufacture a medicament or pharmaceutical composition for administration into a subject in need thereof. For example, the TCR of the antigen-reactive cell can be sequenced to determine the sequence of the paired TCR in the antigen-reactive cell. A polynucleotide comprising the sequence encoding the paired TCR can then be delivered into another host cell, which can be used to manufacture a medicament or pharmaceutical composition for administration into a subject in need thereof. Various delivery methods or vectors described in the present disclosure can be used to deliver the polynucleotide comprising the sequence encoding the paired TCR into another host cell.

The plurality of TCR-expressing cells described herein can express a plurality of TCRs comprising at least about 10, 20, 30, 40, 50, 100, 200, 500, 1,000, 5,000, 10,000, 100,000, 1,000,000, or more different cognate pairs (e.g., natively paired TCRs) derived from the same subject. The plurality of TCRs can further comprise V regions from a plurality of V genes. The plurality of TCR-expressing cells can be engineered cells. The engineered cells can exogenously express the plurality of TCRs.

The cancer cell line used to identify antigen-reactive cells can comprise at least about 50, 100, 1,000, 10,000, 100,000, 1,000,000, 10,000,000, 100,000,000 or more cells.

The enriched subset of the plurality of TCR-expressing cells described herein may be administered directly into the subject in need thereof. In some cases, the enriched subset may not be clear of the cancer cell line and as such may cause issues when administering into the subject. The method can further comprise killing the cancer cell line prior to contacting the cancer cell line with the plurality of TCR-expressing cells. The killing can comprise irradiating or treating the cancer cell line with a chemical compound. The chemical compound can be a cytotoxic compound. Examples of cytotoxic compound include, but are not limited to, the cytotoxic compound is cis-platin, cyclophosphamide, nitrogen mustard, trimethylene thiophosphoramide, carmustine, busulfan, chlorambucil, belustine, uracil mustard, chlomaphazin, dacabazine, cytosine arabinoside, fluorouracil, methotrexate, mercaptopuirine, azathioprime, procarbazine, doxorubicin, bleomycin, dactinomycin, daunorubicin, mithramycin, mitomycin, mytomycin C, daunomycin, or any combination thereof.

In some other aspects, the present disclosure provides methods for identifying an antigen-reactive cell or a TCR of the antigen-reactive cell that recognizes an antigen in complex with an MHC molecule expressed by a subject. For example, the method can comprise providing a cancer cell line expressing an antigen in complex with an exogenous MHC molecule. The exogenous MHC molecule can be the MHC molecule expressed by the subject or derived from the subject. Next, the cancer cell line can be contacted with a plurality of engineered cells expressing a plurality of TCRs comprising at least about 20, 30, 40, 50, 100, 200, 500, 1,000, 5,000, 10,000, 100,000, 1,000,000, or more different cognate pairs derived from the same subject. The plurality or a subset of the plurality of engineered cells can be activated by the antigen in complex with the exogenous MHC of the cancer cell line. Subsequent to contacting, the plurality or the subset of the plurality of engineered cells can be enriched to identify the antigen-reactive cell or the TCR of the antigen-reactive cell.

The antigen can be endogenous to the cancer cell line. The cancer cell line may not express an exogenous antigen or may not present an exogenous antigen. The antigen can be a tumor-associated antigen (TAA) or a tumor-specific antigen (TSA).

The cancer cell line may not be derived from the same subject. The cancer cell line may be derived from another subject, e.g., a healthy donor. The cancer cell line can be any type of cancer cell line described herein. The cancer cell line can have a transcriptomic profile or genomic alteration that resembles a primary cancer cell isolated from the subject.

The plurality of engineered cells can comprise a plurality of TCRs derived from a primary cell (e.g., a primary T cell) isolated from the subject. The primary cell can be a T cell. The T cell can be a tumor-infiltrating T cell. The T cell can be a peripheral T cell. The peripheral T cell can be a tumor-experienced T cell. The peripheral T cell can be a PD-1+ T cell. The T cell can be a CD4+ T cell, a CD8+ T cell, or a combination thereof. The T cell can be a cytotoxic T cell, a memory T cell, a national killer T cell, an alpha beta T cell, a gamma delta T cell, or any combination thereof. The plurality of engineered cells described herein can exogenously express the plurality of TCRs. For example, sequences encoding the plurality of TCRs can be introduced into the engineered cells for expressing the plurality of TCRs.

The plurality or the subset of the plurality of engineered cells can be enriched (e.g., selected or sorted) based on a marker. For example, FACS or MACS can be used to select the cells based on the marker. The marker can be a reporter protein. The reporter protein can be a fluorescent protein. The marker can be a cell surface protein, an intracellular protein or a secreted protein. The marker can be the intracellular protein or the secreted protein, and the method can further comprise, prior to selecting, fixing and/or permeabilizing the plurality of engineered cells. The method can further comprise contacting the plurality of engineered cells with a Golgi blocker. The secreted protein can be a cytokine. The cytokine can be IFN-γ, TNF-alpha, IL-17A, IL-2, IL-3, IL-4, GM-CSF, IL-10, IL-13, granzyme B, perforin, or a combination thereof. The cell surface protein can be CD39, CD69, CD103, CD25, PD-1, TIM-3, OX-40, 4-1BB, CD137, CD3, CD28, CD4, CD8, CD45RA, CD45RO, GITR, FoxP3, or a combination thereof.

The TCR expressed by the antigen-reactive cell can be identified. For example, sequencing can be used to analyze a TCR repertoire of the subset of the plurality of engineered cells and identify the TCR of the antigen-reactive cell. The antigen-reactive cell or a cell comprising the TCR of the antigen-reactive cell can be administered into the subject.

In some cases, the method can further comprise isolating a primary cancer cell from the subject prior to providing the cancer cell line. Transcriptomic or genomic analysis of the primary cancer cell and some candidate cancer cell lines can be conducted to identify the cancer cell line having a transcriptomic profile or genomic alteration that resembles a primary cancer cell isolated from the subject.

The present disclosure also provides a method for evaluating or analyzing an anti-cancer activity of a TCR-expressing cell. For example, in some cases, the method can comprise providing a plurality of cells derived from a cancer cell line. The plurality of cells can express an endogenous antigen in complex with an exogenous MHC molecule. The exogenous MHC molecule can be an MHC molecule expressed by a subject or derived from the subject in need thereof (e.g., a cancer patient). Next, the plurality of cells can be contacted with a plurality of TCR-expressing cells expressing a plurality of TCRs derived from the same subject (e.g., the subject from whom the MHC molecule is derived). Upon contacting, the plurality of TCRs or a fraction thereof can recognize (e.g., interact or bind) the endogenous antigen in complex with the exogenous MHC molecule of the plurality of cells or a fraction thereof. Subsequent to contacting, a fraction of the plurality of cells that are recognized by the plurality of TCR-expressing cells or a fraction thereof can be quantified. For example, the fraction of the plurality of cells that are recognized by the plurality of TCR-expressing cells or a fraction thereof can be quantified by flow cytometry based methods (e.g., FACS or MACS) or optofluidic technology (e.g., commercially available from Berkeley Lights). The fraction of the plurality of cells that are recognized by the plurality of TCR-expressing cells or a fraction thereof may be lysed or dead cells. The fraction of the plurality of cells that are recognized by the plurality of TCR-expressing cells or a fraction thereof can be quantified based on a marker. In some cases, the fraction can be determined by FACS or MACS based on a marker which can be used to label lysed or dead cells. The marker can be related to apoptosis (e.g., caspase 3) or can be a dye for staining lysed or dead cells. In some cases, a lactate dehydrogenase (LDH) assay can be used to determine the amount of LDH released from lysed or dead cells, which can be used to calculate the amount of lysed or dead cells in a sample. In some cases, a fraction (e.g., the activated fraction) of the plurality of TCR-expressing cells that recognize the plurality of cells or a fraction thereof can be quantified, e.g., by flow cytometry based methods or optofluidic technology. The fraction of the plurality of TCR-expressing cells that recognize the plurality of cells or a fraction thereof can be quantified based on a marker. In some cases, the fraction of the plurality of TCR-expressing cells that recognize the plurality of cells or a fraction thereof can be determined by FACS or MACS based on a marker. The marker can be CD39, CD69, CD103, CD25, PD-1, TIM-3, OX-40, 4-1BB, CD137, CD3, CD28, CD4, CD8, CD45RA, CD45RO, GITR, CD107a, TNF-alpha, or FoxP3. As described herein, the optofluidic technology can comprise distributing cells within a sample into individual compartments using microfluidic devices, and detecting a signal associated with the subset of cells with the property of interest. The signal may be generated only within the compartments containing the cells with the property of interest. For example, the signal can be associated with lysed or dead cells when determining the fraction of cells that are recognized by the plurality of TCR-expressing cells or can be associated with secreted cytokines from T cells when determining the fraction of activated T cells. In some cases, an amount or level of a cytokine secreted by the plurality of TCR-expressing cells or a fraction thereof can also be quantified (e.g., cytokine release assay). Examples of cytokines include, but not limited to, IFN-γ, TNF-alpha, IL-17A, IL-2, IL-3, IL-4, GM-CSF, IL-10, IL-13, granzyme B and perforin. Alternatively, the plurality of cells can be APCs expressing an exogenous MHC molecule. The APCs can be professional or non-professional APCs. The APCs can be cancer cell lines described herein, or can be isolated from a subject. The APC can be an autologous APC. The TCR-expressing cells can be any one of the TCR-expressing cells described herein.

### Labeling of Antigen-Reactive Cells

The antigen-reactive cells (e.g., antigen-reactive TCR-expressing cells) can be selected or enriched based on a marker as described herein. The antigen-reactive cells may upregulate a cell surface marker or reporter gene following interaction with an APC such as the cancer cell line described herein due to triggering the TCR signaling pathways. T cells that upregulate the cell surface marker or reporter gene can be quantified (e.g., by fluorescent microscopy or by flow cytometry) or enriched (e.g., by FACS or MACS).

Besides cell markers or reporter genes, other methods can be used to label the antigen-reactive cells, which can then be selected by the methods described herein such as FACS or MACS. In some cases, the antigen-presenting cell (APC) such as the cancer cell line described herein can be engineered to label the TCR-expressing cell that interacts with the APC. For example, the APC may be engineered to be associated with (e.g., express or be attached with) a label-transferring enzyme, which can catalyze the transfer of a label to the TCR-expressing cell that is physically interacting with the APC. The label can be a detectable label. The label can comprise a substrate of the label-transferring enzyme. The label can comprise a detectable moiety. The detectable moiety can be attached to a substrate that can be recognized by the label-transferring enzyme. In some cases, the TCR-expressing cell can be associated with (e.g., express or be attached with) a label-accepting moiety, which can then be attached to the label under the catalyzation of the label-transferring enzyme. The TCR-expressing cell can express the label-accepting moiety endogenously or exogenously. The TCR-expressing cell can be attached to the label-accepting moiety chemically, for example, through a chemical linkage.

A non-limiting example of such label-transferring enzyme can be a transpeptidase. The transpeptidase can be a sortase, such as sortase A (SrtA), sortase B, archaeosortase A, exosortase A, rhombosortase, or PorU. In some cases, the transpeptidase is SrtA, which can be found in the genome of many bacteria such as Staphylococcus aureus. SrtA can use a peptide (e.g., a LPXTG penta-peptide) as the substrate and transfer this substrate to an N-terminal triglycine moiety that is present on the TCR-expressing cell. The SrtA may comprise one or more mutations selected from the group consisting of P94S, D124G, D160N, D165A, Y187L, E189R, K190E, K196T, F200L, and any combination thereof, which can modulate the activity of SrtA.

The N-terminal triglycine moiety can be attached onto the surface of the TCR-expressing cell. For example, a chemically synthesized peptide comprising the N-terminal triglycine moiety can be chemically conjugated to the TCR-expressing cell. An example method for such chemical conjugation may comprise reacting a trans-cyclooctene (TCO) group to a tetrazine group in what is known as copper-free click chemistry. For example, the TCO group can be conjugated to the peptide through a thiol maleimide reaction, and the tetrazine group can be conjugated to the TCR-expressing cell surface using the NHS ester group. Then the TCO-modified peptide can be attached to the cell culture media and react with the tetrazine-modified cell surface. Unreacted TCO-modified peptide can be washed away. It should be understood that the click chemistry used herein may not be limited to copper-free click chemistry. Other click chemistry or other chemical conjugation may be used, including but not limited to azide-alkyne cycloaddition (e.g., copper-catalyzed azide-alkyne cycloaddition and ruthenium-catalyzed azide-alkyne cycloaddition), alkyne-nitrone cycloadditions, alkene and tetrazine inverse-demand Diels-Alder, or alkene and tetrazole photoclick reaction.

The label can comprise a substrate, for example, a substrate peptide. The substrate such as the LPXTG penta-peptide can be modified with a detectable moiety such as biotin, a fluorescent dye, digoxigenin, a peptide tag (e.g., HIS-tag or FLAG-tag), or a conjugation handle. The detectable moiety can be detected by flow cytometry directly or indirectly. For example, the fluorescent dye can be detected directly. For another example, the substrate can be modified with a conjugation handle to which another detectable moiety can be attached through a variety of reactions such as click chemistry reactions, and can be detected indirectly. The detectable moiety can be attached to the substrate prior to the substrate being transferred by the enzyme to the label-accepting moiety. Alternatively, the detectable moiety can be attached after the substrate has been transferred to the label-accepting moiety of the TCR-expressing cell.

The label-transferring enzyme can be expressed on the surface of the APC. For example, the label-transferring enzyme can be fused to a signal peptide (e.g., a N-terminal signal peptide), and in some cases, the label-transferring enzyme can be further fused to a transmembrane domain (e.g., a C-terminal transmembrane domain). In some cases, SrtA can be expressed on the surface of the APC. For example, SrtA can be expressed by fusing SrtA to a signal peptide. The signal peptide can be N-terminal signal peptide such as that of the B2M (MSRSVALAVLALLSLSGLEA). SrtA can be further fused to a transmembrane domain. The transmembrane domain can be a C-terminal transmembrane domain such as that of the alpha chain of a Class I MHC molecule (VGIIAGLVLLGAVITGAVVAAVMW). Various signal peptides or transmembrane domain sequences from other proteins may be used. These sequences may be found in UniProt database. Alternatively, SrtA can also be fused to a scaffold protein, which is a membrane protein or membrane-anchored protein with a known interaction partner. Examples of scaffold protein include, but are not limited to, single-chain antibody, HER2, CD40, CD40L, and many other cell surface proteins. These fusion proteins can be expressed intracellularly inside the APC and transported to the cell surface naturally. When SrtA is fused with a scaffold protein, the TCR-expressing cell may be associated with (e.g., express, engineered to express, or labeled with) the interaction partner of the scaffold protein. The scaffold protein and the interaction partner may be modified (e.g., introducing mutations) so that their interaction alone may not drive the interaction between the APC and the TCR-expressing cell. As a non-limiting example, the scaffold protein can be CD40L and its interaction partner can be CD40; and K142E and R202E mutations can be introduced to CD40L to reduce its affinity to CD40. The interaction partner may also comprise a N-terminal triglycine moiety to accept the LPXTG substrate and the detectable moiety attached to it.

Another non-limiting example of label-transferring enzyme can be glycosyltransferase. The glycosyltransferase can transfer saccharide moieties from a nucleotide sugar substrate (e.g., UDP-glucose, UDP-galactose, UDP-GlcNAc, UDP-GalNAc, UDP-xylose, UDP-glucuronic acid, GDP-mannose, GDP-fucose, and CMP-sialic acid) to a nucleophilic glycosyl accepter molecule. The nucleophilic glycosyl accepter molecule can be oxygen-, carbon-, nitrogen-, or sulfur-based.

For example, the glycosyltransferase can be H. pylori α-1,3-fucosyltransferase (FucT). FucT can transfer the fucose moiety of GDP-fucose, the natural substrate of FucT, to N-acetyllactosamine (LacNAc) or α2,3-sialyl LacNAc which can be found on the surface of many types of mammalian cells including T cells. FucT can also tolerate certain modifications on its substrate. For example, a detectable moiety such as biotin, fluorophore or a conjugation handle can be linked to the fucose moiety of GDP-fucose through the C-5 position on the fucose. Therefore, the FucT can be attached to the surface of a APC, and transfer, for example, the fucose-biotin moiety of the GDP-fucose-biotin to the surface of the TCR-expressing cell that is interacting with the APC. The biotin in this example can also be replaced by many other detectable moieties.

FucT can be attached to the surface of APC using various methods. For example, FucT can be fused with a signaling peptide at its N-terminal signal peptide and C-terminal transmembrane domain, and expressed intracellularly inside the APC, as described above for SrtA. Alternatively, FucT can be produced outside the APC, and attached to the surface of APC biochemically. Various chemistries can be used including the click chemistry described herein. For example, FucT can be modified with TCO using TCO-NHS to form TCO-FucT. The APC can be modified with tetrazine using tetrazine-NHS. Then the TCO-FucT can be contacted with the tetrazine-modified APC to attach FucT to the APC. Alternatively, GDP-fucose-tetrazine may be used to further convert TCO-FucT to GDP-fucose-FucT, where the GDP-fucose moiety and the FucT are linked through the reaction product of tetrazine-TCO click chemistry. GDP-fucose-FucT can be incubated with APC where the FucT moiety can catalyze the reaction of attaching itself (or another GDP-fucose-FucT molecule) to the LacNAc or sialyl LacNAc on the APC. These biochemical methods can also be used to attach SrtA to the surface of APC.

In some cases, the TCR-expressing cell that recognizes the APC can be labeled by co-culturing the APC and the TCR-expressing cells, after which the complex formed by interacting APC and TCR-expressing cell can be captured. For example, such complex can be stabilized using low concentration of fixatives such as 0.1% to 0.5% paraformaldehyde. The complex can be separated from non-interacting APC and TCR-expressing cells based on size, which may manifest as light scattering in flow cytometry and FACS. To facilitate isolation of the complex, the APC and TCR-expressing cell may also be stained with different fluorescent dyes. For example, the APC can be stained with a green dye and the TCR-expressing cell can be stained with a red dye. During FACS, particles possessing both the greed and red dyes (presumed to be complexed formed by the APC and TCR-expressing cell) can be separated from particles possessing dyes of only one color. Using this method, the APC itself can be considered a label attached to the TCR-expressing cell that can recognize the antigen presented by the APC.

With or without the use of the fixative, the complex formed by the APC and TCR-expressing cell can be encapsulated in individual compartments such as water-in-oil droplets in an emulsion. General methods of making such water-in-oil emulsions include but are not limited to microfluidics, vortexing, or shaking. Any of these methods can be used. The density of the APCs and TCR-expressing cells in the aqueous phase before emulsion generation can be controlled to be sufficiently low so that if an APC and a TCR-expressing cell are not interacting at the time of emulsion generation, they may be unlikely to be partitioned in the same droplet. The APC may produce a label which may attach to the TCR-expressing cell in the same droplet even if the TCR-expressing cell dissociates from the APC after emulsification. For example, the APC may secrete a protein capable of binding to the TCR-expressing cell directly or indirectly. For example, the TCR-expressing cell may be first labeled with a bi-specific antibody which recognize a cell surface protein of the T cell (e.g., CD45, CD2) as well as a cytokine (e.g., TNF-alpha). The APC can be engineered to secrete the cytokine recognized by the bi-specific antibody. To reduce the production of the cytokine before emulsion generation, the expression cassette of this cytokine may be under the control of an inducible promoter (e.g., a TetOn promoter), and the inducer (e.g., tetracycline, doxycycline) can be added to the media immediately before emulsification. The emulsion can be demulsified and the cytokine bound to the TCR-expressing cell can serve as a label to quantify or enrich the TCR cells that can recognize the APC.

In some cases, provided herein are methods for identifying an antigen-reactive cell that recognizes an endogenous antigen in complex with an MHC molecule expressed by a subject. The subject may have a condition such as cancer. The method can comprise providing an antigen-presenting cell (APC) expressing an endogenous antigen in complex with an exogenous MHC molecule. The exogenous MHC molecule can be the MHC molecule expressed by the subject or derived from the subject. Next, the APC can be contacted with a plurality of TCR-expressing cells derived from the subject. The plurality of TCR-expressing cells or a subset of the plurality of TCR-expressing cells can recognize the endogenous antigen in complex with the exogenous MHC of the APC. The plurality of TCR-expressing cells or a subset of the plurality of TCR-expressing cells that recognizes the endogenous antigen can be attached to a label secreted from the APC or a label transferred by a label-transferring enzyme associated with the APC upon recognizing the endogenous antigen, or they can express an activation marker upon recognizing the endogenous antigen. Next, the subset of the plurality of TCR-expressing cells based on the label or the activation marker can be identified, thereby identifying the antigen-reactive cell. The identifying can comprise enriching the subset of the plurality of TCR-expressing cells. The APC can express at least about 10, 50, 100, 200, 300 or more endogenous antigens.

The methods provided herein can be used for companion diagnosis. For example, the method can further comprise determining whether to administer a cancer drug to the subject. Determination can be based on a fraction of the subset of the plurality of TCR-expressing cells in the plurality of TCR-expressing cells or the number of the TCR-expressing cells in the subset that recognizes the antigen. The number of the subset of the plurality of TCR-expressing cells can be quantified, e.g., by flow cytometry. Various markers (e.g., cell surface marker or secreted cytokines) disclosed herein that indicate T cell activation can be used. The number of the plurality of TCR-expressing cells prior to contacting with the APC can be quantified. The fraction of the subset of the plurality of TCR-expressing cells in the plurality of TCR-expressing cells can be determined based on the quantification. Whether or not to administer a cancer drug to the subject can be determined based on the fraction or the number of the TCR-expressing cells in the subset. In some cases, a cancer drug can be administered to the subject determined as being suitable for treatment with the cancer drug based on the fraction. In some other cases, a cancer drug may not be administered to the subject determined as being unsuitable for treatment with the cancer drug based on the fraction. The methods can also be used to determine whether to increase or decrease a dose of the cancer drug. In some cases, a dose of the cancer drug to the subject can be increased. In some cases, a dose of the cancer drug to the subject can be decreased.

The cancer drug can be an immune cell regulator. The immune cell regulator can be a cytokine or an immune checkpoint inhibitor.

The methods described herein can further comprise determining a TCR sequence of the subset of the plurality of TCR-expressing cells. Next, a polynucleotide molecule having the TCR sequence can be delivered (e.g., introduced, transformed, transduced or transfected) into a recipient cell for expression. The recipient cell can be a host cell for TCR expression. The recipient cell can be any type of cell disclosed in the “TCR-expressing cell” section. For example, the recipient cell can be a T cell. The T cell can be an autologous T cell or an allogenic T cell. The recipient cell may not comprise the TCR sequence prior to delivering. In some cases, an endogenous TCR of the recipient cell can be inactivated (e.g., knocked out or knocked down). The recipient cell or derivative thereof (e.g., copy or offspring of the recipient cell) can be delivered into the subject, for example, as a treatment.

In some cases, the subset of the plurality of TCR-expressing cells that recognizes the antigen can express at least two different TCRs. The sequences of the at least two different TCRs can be determined. Next, a plurality of polynucleotide molecules comprising the at least two different TCRs can be delivered into a plurality of recipient cells for expression. The recipient cells expressing the TCRs may be further selected. For example, the plurality of recipient cells can be contacted with the APC or an additional APC. Next, a recipient cell from the plurality of recipient cells can be enriched (e.g., by FACS or MACS), which recipient cell recognizes the APC or the additional APC.

The label described herein can comprise a detectable moiety. The detectable moiety can be detectable by flow cytometry. The detectable moiety can be a biotin, a fluorescent dye, a peptide, digoxigenin, or a conjugation handle. The conjugation handle can comprise, for example, an azide, an alkyne, a DBCO, a tetrazine, or a TCO. The label can comprise a substrate recognized by the label-transferring enzyme. The label is a cytokine secreted by the APC. The label-transferring enzyme can be a transpeptidase (e.g., sortase) or a glycosyltransferase (e.g., fucosyltransferase). The label-transferring enzyme can be expressed by the APC or may be supplied outside and attached to the APC. The label-transferring enzyme can be a transmembrane protein. The label-transferring enzyme can be attached to the APC via covalent or non-covalent interaction. The APC can be derived from a subject. The APC can be a cancer cell line described herein. The cancer cell line may be derived from a same cancer type as the cancer of the subject.

The plurality of TCR-expressing cells can comprise T cells. The T cells can be tumor-infiltrating T cells or peripheral T cells. The T cells can express LAG3, CD39, CD69, CD103, CD25, PD-1, TIM-3, OX-40, 4-1BB, CD137, CD3, CD28, CD4, CD8, CD45RA, CD45RO, GITR, or FoxP3, or any combinations thereof. The plurality of TCR-expressing cells can comprise a label-accepting moiety for receiving the label.

It should be understood that the methods provided herein may be used for TCR identification with various APCs not limited to cancer cell lines described herein. The APC can be professional or non-professional APCs. The APC can be a primary cell isolated from a subject such as a healthy subject or a subject having a condition. The APC may be engineered to express a subject-specific MEW. The APC can be cancer-mimicking APC or cmAPC, which may carry similar antigens as a cancer cell. The APC may be a cell line. The APC may not be immortalized.

### TCR-Expressing Cells

In various aspects, a plurality of TCR-expressing cells can be used in the methods described herein for identifying an antigen-reactive cell from the plurality of TCR-expressing cells. The TCR-expressing cells can be primary T cells obtained from a subject or engineered cells expressing subject-derived or subject-specific TCRs. The subject-derived or subject-specific TCRs can be specific to the subject or the tumor of the subject.

The TCR-expressing cells can be T cells. The T cells can be CD4+ T cells, CD8+ T cells, or CD4+/CD8+ T cells. The TCR-expressing cells such as T cells can be obtained from a subject (e.g., primary T cells). The TCR-expressing cells may be obtained from any sample described herein. For example, the sample can be a peripheral blood sample. The peripheral blood cells can be enriched for a particular cell type (e.g., mononuclear cells, red blood cells, CD4+ cells, CD8+ cells, immune cells, T cells, NK cells, or the like). The peripheral blood cells can also be selectively depleted of a particular cell type (e.g., mononuclear cells, red blood cells, CD4+ cells, CD8+ cells, immune cells, T cells, NK cells, or the like).

The T cell can be obtained from a tissue sample comprising a solid tissue, with non-limiting examples including a tissue from brain, liver, lung, kidney, prostate, ovary, spleen, lymph node (e.g., tonsil), thyroid, thymus, pancreas, heart, skeletal muscle, intestine, larynx, esophagus, and stomach. Additional non-limiting sources include bone marrow, cord blood, tissue from a site of infection, ascites, pleural effusion, spleen tissue, and tumors. The T cell can be derived or obtained from a healthy donor, from a patient diagnosed with cancer or from a patient diagnosed with an infection. The T cell can be part of a mixed population of cells which present different phenotypic characteristics.

The T cells can be helper T cells, cytotoxic T cells, memory T cells, regulatory T cells, natural killer T cells, alpha beta T cells, or gamma delta T cells. In certain aspects of the present disclosure, T cells can be obtained from a unit of blood collected from a subject using a variety of techniques, such as Ficoll™ separation. Cells from the circulating blood of an individual can be obtained by apheresis. The apheresis product may contain lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media for subsequent processing steps. In some cases, the cells can be washed with phosphate buffered saline (PBS). The wash solution may lack calcium or magnesium or other divalent cations. Initial activation steps in the absence of calcium can lead to magnified activation. A washing step may be accomplished by methods such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

The TCR-expressing cells can be T cells isolated from a sample and selected with certain properties by various methods. The T cells can be isolated from peripheral blood lymphocytes or tissues by lysing the red blood cells and depleting the monocytes, for example, by centrifugation through a PERCOLL™ gradient or by counterflow centrifugal elutriation. When isolating T cells from tissues (e.g., isolating tumor-infiltrating T cells from tumor tissues), the tissues made be minced or fragmented to dissociate cells before lysing the red blood cells or depleting the monocytes. A specific subpopulation of T cells, such as CD3+, CD28+, CD4+, CD8+, CD45RA+, and CD45RO+ T cells, can be further isolated by positive or negative selection techniques. For example, T cells can be isolated by incubation with anti-CD3/anti-CD28 (e.g., 3×28)-conjugated beads, such as DYNABEADS™ M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least or equal to about 1, 2, 3, 4, 5, or 6 hours. In yet another aspect, the time period is 10 to 24 hours. In an aspect, the incubation time period is about 24 hours. Longer incubation times may be used to isolate T cells in any situation where there are few T cells as compared to other cell types, such as in isolating tumor infiltrating lymphocytes (TILs) from tumor tissue or from immunocompromised individuals. Further, use of longer incubation times can increase the efficiency of capture of CD8+ T cells. Thus, by simply shortening or lengthening the time T cells are allowed to bind to the anti-CD3/anti-CD28 beads and/or by increasing or decreasing the ratio of beads to T cells, subpopulations of T cells can be selected for or against at culture initiation or at other time points during the process. Additionally, by increasing or decreasing the ratio of anti-CD3 and/or anti-CD28 antibodies on the beads or other surface, subpopulations of T cells can be selected for or against at culture initiation or at other desired time points. In some cases, multiple rounds of selection can be used. In certain aspects, the selection procedure can be performed and the “unselected” cells (cells that may not bind to the anti-CD3/anti-CD28 beads) can be used in the activation and expansion process. “Unselected” cells can also be subjected to further rounds of selection.

Enrichment of a T cell population by negative selection can be accomplished with a combination of antibodies directed to surface markers unique to the negatively selected cells. An example method can be cell sorting and/or selection via negative magnetic immune adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain aspects, it may be useful to enrich for or positively select for regulatory T cells which typically express CD4+, CD25+, CD62Lhi, GITR+, and FoxP3+. Alternatively, in certain aspects, T regulatory cells are depleted by anti-C25 conjugated beads or other similar method of selection.

A T cell population can be selected that expresses one or more of IFN-γ, TNF-alpha, IL-17A, IL-2, IL-3, IL-4, GM-CSF, IL-10, IL-13, granzyme B, and perforin, or other molecules, e.g., other cytokines and transcription factors such as T-bet, Eomes, Tcf1 (TCF7 in human). Methods for screening for cell expression can be determined, e.g., by the methods described in PCT Publication No.: WO 2013/126712.

For isolation of a population of cells by positive or negative selection, the concentration of cells and surface (e.g., particles such as beads) can be varied. In certain aspects, the volume in which beads and cells are mixed together may be decreased (e.g., increase the concentration of cells) to ensure maximum contact of cells and beads. For example, in an aspect, a concentration of 2 billion cells/mL is used. In another aspect, a concentration of 1 billion cells/mL is used. In a further aspect, greater than 100 million cells/mL is used. In a further aspect, a concentration of cells of at least about 10, 15, 20, 25, 30, 35, 40, 45, or 50 million cells/mL is used. In some aspects, a concentration of cells of at least about 75, 80, 85, 90, 95, or 100 million cells/mL is used. In some aspects, a concentration of cells of at least about 125 or 150 million cells/mL can be used. Using high concentrations can result in increased cell yield, cell activation, and cell expansion. Further, use of high cell concentrations can allow more efficient capture of cells that may weakly express cell surface markers of interest, such as CD28-negative T cells, or from samples where there are many tumor cells present (e.g., leukemic blood, tumor tissue, etc.). Such populations of cells may have therapeutic value. For example, using high concentration of cells can allow more efficient selection of CD8+ T cells that may have weaker CD28 expression.

In some cases, lower concentrations of cells may be used. By significantly diluting the mixture of T cells and surface interactions between the particles and cells can be minimized. This can select for cells that express high amounts of desired antigens to be bound to the particles. For example, CD4+ T cells can express higher levels of CD28 and can be more efficiently captured than CD8+ T cells in dilute concentrations. In some aspects, the concentration of cells used is at least about 5×10⁵/mL, 5×10⁶/mL, or more. In other aspects, the concentration used can be from about 1×10⁵/mL to 1×10⁶/mL, and any integer value in between. In other aspects, the cells may be incubated on a rotator for varying lengths of time at varying speeds at either 2-10° C. or at room temperature.

T cells can also be frozen after a washing step. The freeze and subsequent thaw step may provide a more uniform product by removing granulocytes and to some extent monocytes in the cell population. After the washing step that removes plasma and platelets, the cells may be suspended in a freezing solution. While many freezing solutions and parameters may be useful in this context, one method that can be used involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing Hespan and PlasmaLyte A. The cells can then be frozen to −80° C. and stored in the vapor phase of a liquid nitrogen storage tank. Cell may be frozen by uncontrolled freezing immediately at −20° C. or in liquid nitrogen. In certain aspects, cryopreserved cells are thawed and washed and allowed to rest for one hour at room temperature prior to use.

Also contemplated in the context of the present disclosure is the collection of blood samples or apheresis product from a subject at a time period prior to when cells (e.g., TCR-expressing cells) might be needed. In some cases, a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present disclosure, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Thus, it is contemplated within the context of the present disclosure to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Besides primary T cells obtained from a subject, the TCR-expressing cells may be cell-line cells, such as cell-line T cells. Examples of cell-line T cells include, but are not limited to, Jurkat, CCRF-CEM, HPB-ALL, K-T1, TALL-1, MOLT 16/17, and HUT 78/H9.

The TCR-expressing cell can be a T cell obtained from an in vitro culture. T cells can be activated or expanded in vitro by contacting with a tissue or a cell. See “Activation and Expansion” section. For example, the T cells isolated from a patient's peripheral blood can be co-cultured with cells presenting tumor antigens such as tumor cells, tumor tissue, tumorsphere, tumor lysate-pulsed APC or tumor mRNA-loaded APC. The cells presenting tumor antigens may be APC pulsed with or engineered to express a defined antigen, a set of defined antigens or a set of undefined antigens (such as tumor lysate or total tumor mRNA). For example, in the cases of presenting defined antigens, an APC can express one or more minigenes encoding one or more short epitopes (e.g., from 7 to 13 amino acids in length) with known sequences. An APC can also express two or more minigenes from a vector containing sequences encoding the two or more epitopes. In the cases of presenting undefined antigens, an APC can be pulsed with tumor lysate or total tumor mRNA. The cells presenting tumor antigens may be irradiated before the co-culture. The co-culture may be in media comprising reagents (e.g., anti-CD28 antibody) that may provide co-stimulation signal or cytokines. Such co-culture may stimulate (e.g., activate) and/or expand tumor antigen-reactive T cells. These cells may be selected or enriched using cell surface markers described herein (e.g., CD25, CD69, CD137). Using this method, tumor antigen-reactive T cells can be pre-enriched from the peripheral blood of the patient. These pre-enriched T cells can be used as the TCR-expressing cells in the methods described herein. The pre-enriched T cells (e.g., CD137+) may contain T cells that acquired marker (e.g., CD137) expression during the co-culture, and may also contain T cells that already express the marker at blood draw. The latter population may nevertheless be tumor reactive. This method can offer an easier alternative to isolating tumor-infiltrating lymphocytes (TILs) described.

The TCR-expressing cell can be a tumor-infiltrating lymphocyte (TIL), e.g., tumor-infiltrating T cells. A TIL can be isolated from an organ afflicted with a cancer. One or more cells can be isolated from an organ with a cancer that can be a brain, heart, lungs, eye, stomach, pancreas, kidneys, liver, intestines, uterus, bladder, skin, hair, nails, ears, glands, nose, mouth, lips, spleen, gums, teeth, tongue, salivary glands, tonsils, pharynx, esophagus, large intestine, small intestine, rectum, anus, thyroid gland, thymus gland, bones, cartilage, tendons, ligaments, suprarenal capsule, skeletal muscles, smooth muscles, blood vessels, blood, spinal cord, trachea, ureters, urethra, hypothalamus, pituitary, pylorus, adrenal glands, ovaries, oviducts, uterus, vagina, mammary glands, testes, seminal vesicles, penis, lymph, lymph nodes or lymph vessels. One or more TILs can be from a brain, heart, liver, skin, intestine, lung, kidney, eye, small bowel, or pancreas. TILs can be from a pancreas, kidney, eye, liver, small bowel, lung, or heart. The one or more cells can be pancreatic islet cells, for example, pancreatic β cells. In some cases, a TIL can be from a gastrointestinal cancer. A TIL culture can be prepared a number of ways. For example, a tumor can be trimmed from non-cancerous tissue or necrotic areas. A tumor can then be fragmented to about 2-3 mm in length. In some cases, a tumor can be fragmented from about 0.5 mm to about 5 mm in size, from about 1 mm to about 2 mm, from about 2 mm to about 3 mm, from about 3 mm to about 4 mm, or from about 4 mm to about 5 mm. Tumor fragments can then be cultured in vitro utilizing media and a cellular stimulating agent such as a cytokine. In some cases, IL-2 can be utilized to expand TILs from a tumor fragment. A concentration of IL-2 can be about 6000 IU/mL. A concentration of IL-2 can also be about 2000 IU/mL, 3000 IU/mL, 4000 IU/mL, 5000 IU/mL, 6000 IU/mL, 7000 IU/mL, 8000 IU/mL, 9000 IU/mL, or up to about 10000 IU/mL. Once TILs are expanded, they can be subject to in vitro assays to determine tumor reactivity. For example, TILs can be evaluated by FACs for CD3, CD4, CD8, and CD58 expression. TILs can also be subjected to cocultured, cytotoxicity, ELISA, or ELISPOT assays. In some cases, TIL cultures can be cryopreserved or undergo a rapid expansion. A cell, such as a TIL, can be isolated from a donor of a stage of development including, but not limited to, fetal, neonatal, young and adult.

The TCR-expressing cells can be T cells, B cells, NK cells, macrophages, neutrophils, granulocytes, eosinophils, red blood cells, platelets, stem cells, iPSCs, mesenchymal stem cells, or an engineered from thereof. In addition, the TCR-expressing cell can be a cell line cell. The cell line can be tumorigenic or artificially immortalized cell line. Examples of cell lines include, but are not limited to, CHO-K1 cells, HEK293 cells, Caco2 cells, U2-OS cells, NIH 3T3 cells, NSO cells, SP2 cells, CHO-S cells, DG44 cells, K-562 cells, U-937 cells, MRCS cells, IMIt90 cells, Jurkat cells, HepG2 cells, HeLa cells, HT-1080 cells, HCT-116 cells, Hu-h7 cells, Huvec cells, and Molt 4 cells. The TCR-expressing cell can be an autologous T cell or an allogeneic T cell.

The TCR-expressing cells can be an engineered cell. The engineered cell can be an engineered T cell. The engineered cell can express an exogenous molecule (e.g., a TCR). The engineered cell can be genetically modified to express a subject-derived or subject-specific TCR. The engineered cell can be genetically modified to express a subject-derived or subject-specific TCR expressed by a primary T cell obtained from the subject having a condition (e.g., cancer). The engineered cell can be a primary cell (e.g., primary T cell obtained from various sources including a healthy donor) genetically modified to express a subject-derived or subject-specific TCR of a subject having a condition. For example, a primary T cell can be obtained from a healthy donor and engineered to express a TCR of a patient having a cancer. The primary T cell can be isolated from a blood sample from the healthy donor. The primary T cell can be a peripheral T cell. The primary T cell can be obtained from various sources or by various methods described herein. The engineered cells can be other types of cells obtained from a subject, including but not limited to B cells, NK cells, macrophages, neutrophils, granulocytes, eosinophils, red blood cells, platelets, stem cells, iPSCs, and mesenchymal stem cells. The engineered cell can be a cell line cell described herein. In some cases, a library of TCR-expressing cells that are engineered cells can be used in the methods described herein for TCR identification. The library can be a synthetic library, where each cell of the engineered cells within the synthetic library exogenously expresses a TCR. The TCR expressed by the engineered cell can be a subject-derived or subject-specific TCR. In some cases, the TCR-expressing cells such as T cells can comprise an endogenous TCR, and the endogenous TCR can be inactivated (e.g., knocked out or knocked down).

A polynucleotide or a sequence encoding a subject-derived or subject-specific TCR may be delivered into a cell for expression. A polynucleotide encoding a subject-derived or subject-specific TCR may be delivered into a cell as a linear or circular nucleic acid molecule to generate the engineered cell. In some cases, the polynucleotide can be delivered (e.g., electroporated, transfected, transduced or transformed) into a cell by electroporation. In some cases, the polynucleotide can be delivered into a cell by a carrier such as a cationic polymer. In some cases, a vector comprising a sequence encoding a subject-derived or subject-specific TCR can be delivered into a cell. In some cases, the subject-derived or subject-specific TCR can be expressed in the cell. The TCR can be expressed from a vector (or an expression vector) such as plasmid, transposon (e.g., Sleeping Beauty, Piggy Bac), and a viral vector (e.g., adenoviral vector, AAV vector, retroviral vector and lentiviral vector). Additional examples of a vector include a shuttle vector, a phagemide, a cosmid and an expression vector. Non-limiting examples of plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some cases, a vector is a nucleic acid molecule as introduced into a cell, thereby producing a transformed cell (e.g., an engineered cell). A vector may include nucleic acid sequences that permit it to replicate in a cell, such as an origin of replication. A vector may also include one or more selectable marker genes and other genetic elements. A vector can be an expression vector that includes a paired TCR-encoding polynucleotide according to the present disclosure operably linked to sequences allowing for the expression of the TCR. A vector can be a viral or a non-viral vector, such a retroviral vector (including lentiviral vectors), adenoviral vectors including replication competent, replication deficient and gutless forms thereof, adeno-associated virus (AAV) vectors, simian virus 40 (SV-40) vectors, bovine papilloma vectors, Epstein-Barr vectors, herpes vectors, vaccinia vectors, Moloney murine leukemia vectors, Harvey murine sarcoma virus vectors, murine mammary tumor virus vectors, Rous sarcoma virus vectors and non-viral plasmids.

In some cases, the vector is a self-amplifying RNA replicon, also referred to as self-replicating (m)RNA, self-replication (m)RNA, self-amplifying (m)RNA, or RNA replicon. The self-amplifying RNA replicon is an RNA that can replicate itself. In some embodiments, the self-amplifying RNA replicon can replicate itself inside of a cell. In some embodiments, the self-amplifying RNA replicon encodes an RNA polymerase and a molecule of interest. The RNA polymerase may be a RNA-dependent RNA polymerase (RDRP or RdRp). The self-amplifying RNA replicon may also encode a protease or an RNA capping enzyme. In some embodiments, the self-amplifying RNA replicon vector is of or derived from the Togaviridae family of viruses known as alphaviruses which can include Eastern Equine Encephalitis virus (EEE), Venezuelan Equine Encephalitis virus (VEE), Everglades virus, Mucambo virus, Pixuna virus, Western Equine Encephalitis virus (WEE), Sindbis virus, South African Arbovirus No. 86, Semliki Forest virus, Middelburg virus, Chikungunya virus, Onyong-nyong virus, Ross River virus, Barmah Forest Virus, Getah Virus, Sagiyama virus, Bebaru virus, Mayaro virus, Una virus, Aura virus, Whataroa virus, Babanki virus, Kyzylagach virus, Highlands J Virus, Fort Morgan virus, Ndumu virus, Buggy Creek virus, and any other virus classified by the International Committee on Taxonomy of Viruses (ICTV) as an alphavirus. In some embodiments, the self-amplifying RNA replicon is or contains parts from an attenuated form of the alphavirus, such as the VEE TC-83 vaccine strain. In some embodiments, the self-amplifying RNA replicon vector is an attenuated form of the virus that allows for expression of the molecules of interests without cytopathic or apoptotic effects to the cell. In some embodiments, the self-amplifying RNA replicon vector has been engineered or selected in vitro, in vivo, ex vivo, or in silica for a specific function (e.g., prolonged or increased TCR expression) in the host cell, target cell, or organism. For example, a population of host cells harboring different variants of the self-amplifying RNA replicon can be selected based on the expression level of one or more molecules of interested (encoded in the self-amplifying RNA replicon or in the host genome) at different time point. In some embodiments, the selected or engineered self-amplifying RNA replicon has been modified to reduce the type I interferon response, the innate antiviral response, or the adaptive immune response from the host cell or organism which results in the RNA replicon's protein expression persisting longer or expressing at higher levels in the host cell, target cell, or organism. In some embodiments, this optimized self-amplifying RNA replicon sequence is obtained from an individual cell or population of cells with the desired phenotypic trait (e.g., higher or more sustained expression of the molecules of interest, or reduced innate antiviral immune response against the vector compared to the wildtype strains or the vaccine strains). In some embodiments, the cells harboring the desired or selected self-amplifying RNA replicon sequence are obtained from a subject (e.g., a human or an animal) with beneficial response characteristics (e.g., an elite responder or subject in complete remission) after being treated with a therapeutic agent comprising a self-amplifying RNA replicon. In some embodiments, the self-amplifying RNA replicon vector can express additional agents. In some embodiments, the additional agents include cytokines such as IL-2, IL-12, IL-15, IL-10, GM-CSF, TNF alpha, granzyme B, or a combination thereof. In some embodiments, the additional agent is capable of modulating the expression of the TCR, either by directly affecting the expression of the TCR or by modulating the host cell phenotype (e.g., inducing apoptosis or expansion). In some embodiments, the self-amplifying RNA replicon can contain one or more sub-genomic sequence(s) to produce one or more sub-genomic polynucleotide(s). In some embodiments, the sub-genomic polynucleotides act as functional mRNA molecules for translation by the cellular translation machinery. A sub-genomic polynucleotide can be produced via the function of a defined sequence element (e.g., a sub-genomic promoter or SGP) on the self-amplifying RNA replicon that directs a polymerase to produce the sub-genomic polynucleotide from a sub-genomic sequence. In some embodiments, the SGP is recognized by an RNA-dependent RNA polymerase (RDRP or RdRp). In some embodiments, multiple SGP sequences are present on a single self-amplifying RNA replicon and can be located upstream of sub-genomic sequence encoding for a TCR, a constituent of the TCR, or an additional agent. In some embodiments, the nucleotide length or composition of the SGP sequence can be modified to alter the expression characteristics of the sub-genomic polynucleotide. In some embodiments, non-identical SGP sequences are located on the self-amplifying RNA replicon such that the ratios of the corresponding sub-genomic polynucleotides are different from instances where the SGP sequences are identical. In some embodiments, non-identical SGP sequences direct the production of a TCR and an additional agent (e.g., a cytokine) such that they are produced at a ratio relative to one another that leads to increased expression of the TCR, increased or faster expansion of the target cell without cytotoxic effects to the target cell or host, or dampens the innate or adaptive immune response against the RNA replicon. In some embodiments, the location of the sub-genomic sequences and SGP sequences relative to one another and the genomic sequence itself can be used to alter the ratio of sub-genomic polynucleotides relative to one another. In some embodiments, the SGP and sub-genomic sequence encoding the TCR can be located downstream of an SGP and sub-genomic region encoding the additional agent such that the expression of the TCR is substantially increased relative to the additional agent. In some embodiments, the RNA replicon or SGP has been selected or engineered to express an optimal amount of the cytokine such that the cytokine promotes the expansion of the T cell or augments the therapeutic effect of the TCR but does not cause severe side effects such as cytokine release syndrome, cytokine storm, or neurological toxicity.

The various vectors described herein can be used to deliver or introduce other genes of interest (e.g., nucleic acids encoding WIC molecules) disclosed in the present disclosure into a host cell.

In some embodiments, provided herein is a vector comprising a paired TCR-encoding polynucleotide encoding a TCRα chain and a TCRβ chain. In some embodiments, provided herein is a vector comprising a paired TCR-encoding polynucleotide encoding a TCRγ chain and a TCR chain. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccina virus, hepatitis B virus, human papillomavirus or a pseudotype thereof. In some embodiments, the vector is a non-viral vector. In some embodiments, the non-viral vector can be formulated into a nanoparticle, a cationic lipid, a cationic polymer, a metallic nanopolymer, a nanorod, a liposome, a micelle, a microbubble, a cell-penetrating peptide, or a liposphere.

The expression of the two TCR chains can be driven by two promoters or by one promoter. In some cases, two promoters are used. In some cases, the two promoters, along with their respective protein-coding sequences for the two chains, can be arranged in a head-to-head, a head-to-tail, or a tail-to-tail orientation. In some cases, one promoter is used. The two protein-coding sequences can be linked, optionally in frame, such that one promoter can be used to express both chains. And in such cases, the two protein-coding sequences can be arranged in a head-to-tail orientation and can be connected with ribosome binding site (e.g., internal ribosomal binding site or IRES), protease cleavage site, or self-processing cleavage site (such as a sequence encoding a 2A peptide) to facilitate bicistronic expression. In some cases, the two chains can be linked with peptide linkers so that the two chains can be expressed as a single-chain polypeptide. Each expressed chain may contain the full variable domain sequence including the rearranged V(D)J gene. Each expressed chain may contain the full variable domain sequence including CDR1, CDR2, and CDR3. Each expressed chain may contain the full variable domain sequence including FR1, CDR1, FR2, CDR2, FR3, and CDR3. In some cases, each expressed chain may further contain a constant domain sequence.

To create expression vectors, additional sequences may be added to the sequence encoding the gene of interest such as the TCR. These additional sequences can include vector backbone (e.g., elements for the vector's replication in target cell or in temporary host such as E. coli), promoters, IRES, sequence encoding the self-cleaving peptide, terminators, accessory genes (such as payloads), as well as partial sequences of the paired TCR-encoding polynucleotides (such as part of the sequences encoding the constant domains).

Protease cleavage sites include, but are not limited to, an enterokinase cleavage site: (Asp)4Lys; a factor Xa cleavage site: Ile-Glu-Gly-Arg; a thrombin cleavage site, e.g., Leu-Val-Pro-Arg-Gly-Ser; a renin cleavage site, e.g., His-Pro-Phe-His-Leu-Val-Ile-His; a collagenase cleavage site, e.g., X-Gly-Pro (where X is any amino acid); a trypsin cleavage site, e.g., Arg-Lys; a viral protease cleavage site, such as a viral 2A or 3C protease cleavage site, including, but not limited to, a protease 2A cleavage site from a picornavirus, a Hepatitis A virus 3C cleavage site, human rhinovirus 2A protease cleavage site, a picornavirus 3 protease cleavage site; and a caspase protease cleavage site, e.g., DEVD recognized and cleaved by activated caspase-3, where cleavage occurs after the second aspartic acid residue. In some embodiments, the present disclosure provides an expression vector comprising a protease cleavage site, wherein the protease cleavage site comprises a cellular protease cleavage site or a viral protease cleavage site. In some embodiments, the first protein cleavage site comprises a site recognized by furin; VP4 of IPNV; tobacco etch virus (TEV) protease; 3C protease of rhinovirus; PC5/6 protease; PACE protease, LPC/PC7 protease; enterokinase; Factor Xa protease; thrombin; genenase I; MMP protease; Nuclear inclusion protein a(N1a) of turnip mosaic potyvirus; NS2B/NS3 of Dengue type 4 flaviviruses, NS3 protease of yellow fever virus; ORF V of cauliflower mosaic virus; KEX2 protease; CB2; or 2A. In some embodiments, the protein cleavage site is a viral internally cleavable signal peptide cleavage site. In some embodiments, the viral internally cleavable signal peptide cleavage site comprises a site from influenza C virus, hepatitis C virus, hantavirus, flavivirus, or rubella virus.

A suitable IRES element to include in the vector of the present disclosure can comprise an RNA sequence capable of engaging a eukaryotic ribosome. In some embodiments, an IRES element of the present disclosure is at least about 250 base pairs, at least about 350 base pairs, or at least about 500 base pairs. An IRES element of the present disclosure can be derived from the DNA of an organism including, but not limited to, a virus, a mammal, and a Drosophila. In some cases, a viral DNA from which an IRES element is derived includes, but is not limited to, picornavirus complementary DNA (cDNA), encephalomyocarditis virus (EMCV) cDNA and poliovirus cDNA. Examples of mammalian DNA from which an IRES element is derived includes, but is not limited to, DNA encoding immunoglobulin heavy chain binding protein (BiP) and DNA encoding basic fibroblast growth factor (bFGF). An example of Drosophila DNA from which an IRES element is derived includes, but is not limited to, an Antennapedia gene from Drosophila melanogaster. Addition examples of poliovirus IRES elements include, for instance, poliovirus IRES, encephalomyocarditis virus IRES, or hepatitis A virus IRES. Examples of flaviviral IRES elements include hepatitis C virus IRES, GB virus B IRES, or a pestivirus IRES, including but not limited to bovine viral diarrhea virus IRES or classical swine fever virus IRES.

Examples of self-processing cleavage sites include, but are not limited to, an intein sequence; modified intein; hedgehog sequence; other hog-family sequence; a 2A sequence, e.g., a 2A sequence derived from Foot and Mouth Disease Virus (FMDV); and variations thereof for each.

A vector for recombinant gene expression (e.g., TCR expression) may include any number of promoters, wherein the promoter is constitutive, regulatable or inducible, cell type specific, tissue-specific, or species specific. Further examples include tetracycline-responsive promoters. The vector can be a replicon adapted to the host cell in which the TCR is to be expressed, and it can comprise a replicon functional in a bacterial cell as well, for example, Escherichia coli. The promoter can be constitutive or inducible, where induction is associated with the specific cell type or a specific level of maturation, for example. Alternatively, a number of viral promoters can be suitable. Examples of promoters include the β-actin promoter, SV40 early and late promoters, immunoglobulin promoter, human cytomegalovirus promoter, retrovirus promoter, elongation factor 1A (EF-1A) promoter, phosphoglycerate kinase (PGK) promoter, and the Friend spleen focus-forming virus promoter. The promoters may or may not be associated with enhancers, wherein the enhancers may be naturally associated with the particular promoter or associated with a different promoter.

Promoters used in mammalian cells can be constitutive (Herpes virus TK promoter; SV40 early promoter; Rous sarcoma virus promoter; cytomegalovirus promoter; mouse mammary tumor virus promoter) or regulated (metallothionein promoter, for example). Vectors can be based on viruses that infect particular mammalian cells, e.g., retroviruses, vaccinia and adenoviruses and their derivatives. Promoters can include, without limitation, cytomegalovirus, adenovirus late, and the vaccinia 7.5K promoters. Enolase is an example of a constitutive yeast promoter, and alcohol dehydrogenase is an example of regulated promoter. The selection of the specific promoters, transcription termination sequences and other optional sequences, such as sequences encoding tissue specific sequences, can be determined by the type of cell in which expression is carried out.

The TCR expressed from the TCR-expressing vectors may be in their natural form or may be in an engineered form. In some cases, the engineered form is a single-chain TCR fragment. In some cases, the engineered form is a TCR-CAR. Existing methods can also be used to introduce functional sequences (e.g., linkers, CD28 TM domains) to paired TCR-encoding polynucleotide in order to create TCR-expressing vectors that express these engineered forms of TCRs. In some cases, polynucleotides encoding one or more additional subunits of a TCR complex may be delivered into a cell to generate the engineered cell. The one or more additional subunits can comprise CD3 epsilon, CD3 beta, CD3 gamma, CD3 zeta, or any combinations thereof.

### Activation and Expansion

The TCR-expressing cell can be a T cell. The T cell can be expanded or stimulated by contact with an agent that stimulates a CD3 TCR complex and a co-stimulatory molecule on the surface of the T cells to create an activation signal for the T cell. The activation and/or expansion can be performed prior to contacting the TCR-expressing cells with the antigen-presenting cell (e.g., the cancer cell line described herein. For example, chemicals such as calcium ionophore A23187, phorbol 12-myristate 13-acetate (PMA), or mitogenic lectins like phytohemagglutinin (PHA) can be used to create an activation signal for the T cell. As non-limiting examples, T cell populations may be stimulated in vitro such as by contact with an anti-CD3 antibody or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. For example, the agents providing each signal may be in solution or coupled to a surface. The ratio of particles to cells may depend on particle size relative to the target cell. In further embodiments, the cells, such as T cells, are combined with agent-coated beads, the beads and the cells are subsequently separated, and then the cells are cultured. In an alternative embodiment, prior to culture, the agent-coated beads and cells are not separated but are cultured together. Conditions appropriate for T cell culture can include an appropriate media (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-g, IL-4, IL-7, GM-CSF, IL-10, IL-2, IL-15, TGFβ, and TNF-α or any other additives for the growth of cells. Other additives for the growth of cells include, but are not limited to, surfactant, plasmanate, and reducing agents such as N-acetyl-cysteine and 2-mercaptoethanoi. Media can include RPMI 1640, A1M-V, DMEM, MEM, a-MEM, F-12, X-Vivo 1, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. The target cells can be maintained under conditions necessary to support growth, for example, an appropriate temperature (e.g., 37° C.) and atmosphere (e.g., air plus 5% CO₂). T cells that have been exposed to varied stimulation times may exhibit different characteristics. The T cell can be activated or expanded by co-culturing with tissue or cells. The cells used to activate T cells can be APC or artificial APC (aAPC).

In some cases, stimulation of T cells can be performed with antigen and irradiated, histocompatible APCs, such as feeder PBMCs. In some cases, cells can be grown using non-specific mitogens such as PHA and allogenic feeder cells. Feeder PBMCs can be irradiated at 40Gy. Feeder PBMCs can be irradiated from about 10 Gy to about 15 Gy, from about 15 Gy to about 20 Gy, from about 20Gy to about 25 Gy, from about 25 Gy to about 30 Gy, from about 30 Gy to about 35 Gy, from about 35 Gy to about 40 Gy, from about 40 Gy to about 45 Gy, from about 45 Gy to about 50 Gy. In some cases, a control flask of irradiated feeder cells only can be stimulated with anti-CD3 and IL-2.

### Antigens

The cancer cell line described herein can comprise (e.g., express or represent) an antigen. The antigen can be a target antigen. The antigen can be a tumor antigen. The antigen can be an endogenous antigen to the cancer cell line. The antigen may be same or different from an antigen expressed by a cancer cell. The cancer cell line can comprise (e.g., express) at least about 5, 10, 20, 50, 100, 200, 300, 400, 500, 600, 700, 800, 900, 1,000 or more endogenous antigens. The cancer cell line can represent the endogenous antigen. In some cases, the cancer cell line can represent the endogenous antigen in complex with an exogenous MHC. For example, the exogenous MHC can be derived from a subject in need of a treatment.

In some cases, the antigen-presenting cell (APC) described herein can comprise an antigen. The antigen can be an endogenous antigen to the APC. The APC can comprise (e.g., express) at least about 5, 10, 20, 50, 100, 200, 300, 400, 500, 600, 700, 800, 900, 1,000 or more endogenous antigens. The APC can be an autologous APC.

The antigen or endogenous antigen of the cancer cell line described herein can include a tumor-specific antigen, a tumor-associated antigen, an embryonic antigen on tumor, a tumor-specific membrane antigen, a tumor-associated membrane antigen, a growth factor receptor, a growth factor ligand, or any other type of antigen that is associated with a cancer. The tumor antigen can be a tumor-specific antigen (TSA). The term “TSA,” as used herein, refers to an antigen that is unique to tumor cells and does not occur on other cells in the body. The tumor antigen can be a tumor-associated antigen (TAA). The term “TAA,” as used herein, refers to an antigen that is not unique to a tumor cell and is also expressed on a normal cell. The expression of the antigen on the tumor can occur under conditions that enable the immune system to respond to the antigen. The TAA may be expressed at much higher levels on tumor cells. The TAA can be determined by sequencing a patient's tumor cells and identifying mutated proteins only found in the tumor. These antigens are referred to as “neoantigens.” The tumor antigen can be an epithelial cancer antigen, a prostate specific cancer antigen (PSA) or prostate specific membrane antigen (PSMA), a bladder cancer antigen, a lung cancer antigen, a colon cancer antigen, an ovarian cancer antigen, a brain cancer antigen, a gastric cancer antigen, a renal cell carcinoma antigen, a pancreatic cancer antigen, a liver cancer antigen, an esophageal cancer antigen, a head and neck cancer antigen, a colorectal cancer antigen, a lymphoma antigen, a B-cell lymphoma cancer antigen, a leukemia antigen, a myeloma antigen, an acute lymphoblastic leukemia antigen, a chronic myeloid leukemia antigen, or an acute myelogenous leukemia antigen. Examples of antigens include, but are not limited to, 1GH-IGK, 43-9F, 5T4, 791Tgp72, 9D7, acyclophilin C-associated protein, alpha-fetoprotein (AFP), α-actinin-4, A3, antigen specific for A33 antibody, ART-4, B7, Ba 733, BAGE, BCR-ABL, beta-catenin, beta-HCG, BrE3-antigen, BCA225, BING-4, BRCA1/2, BTAA, CA125, CA 15-3\CA 27.29\BCAA, CA195, CA242, CA-50, calcium activated chloride channel 2, CAGE, CAM43, CAMEL, CAP-1, carbonic anhydrase IX, c-Met, CA19-9, CA72-4, CAM 17.1, CASP-8/m, CCCL19, CCCL21, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD68, CD70, CD70L, CD74, CD79a, CD79b, CD80, CD83, CD95, CD126, CD132, CD133, CD138, CD147, CD154, CDC27, CDK4, CDK4m, CDKN2A, CML6/6, CO-029, CTLA4, CXCR4, CXCR7, CXCL12, cyclin B, HIF-1a, colon-specific antigen-p (CSAp), CEA (CEACAMS), CEACAM6, c-Met, DAM, E2A-PRL, EGFR, EGFRvIII, EGP-1 (TROP-2), EGP-2, ELF2-M, Ep-CAM, EphA3, fibroblast growth factor (FGF), FGF-5, fibronectin, Flt-1, Flt-3, folate receptor, G250 antigen, Ga733VEpCAM, GAGE, gp100, GRO-β, H4-RET, HLA-DR, HM1.24, human chorionic gonadotropin (HCG) and its subunits, HMGB-1, hypoxia inducible factor (HIF-1), HSP70-2M, HST-2, HTgp-175, Ia, IGF-1R, IFN-γ, IFN-α, IFN-β, IFN-λ, IL-4R, IL-6R, IL-13R, IL-15R, IL-17R, IL-18R, IL-2, IL-6, IL-8, IL-12, IL-15, IL-17, IL-18, IL-23, IL-25, immature laminin receptor, insulin-like growth factor-1 (IGF-1), KC4-antigen, KSA, KS-1-antigen, KS1-4, LAGE-1a, Le-Y, LDR/FUT, M344, MA-50, macrophage migration inhibitory factor (MIF), MAGE, MAGE-1, MAGE-3, MAGE-4, MAGE-5, MAGE-6, MART-1, MART-2, TRAG-3, MC1R, mCRP, MCP-1, mesothelin, MIP-1A, MIP-1B, MIF, MG7-Ag, MOV18, MUC1, MUC2, MUC3, MUC4, MUCSac, MUC13, MUC16, MUM-1/2, MUM-3, MYL-RAR, NB/70K, Nm23H1, NuMA, NCA66, NCA95, NCA90, NY-ESO-1, P polypeptide, p15, p16, p53, p185erbB2, p180erbB3, PAM4 antigen, pancreatic cancer mucin, PD1 receptor (PD-1), PD-1 receptor ligand 1 (PD-L1), PD-1 receptor ligand 2 (PD-L2), PI5, placental growth factor, p53, PLAGL2, Pme117 prostatic acid phosphatase, PSA, PRAME, PSMA, P1GF, ILGF, ILGF-1R, IL-6, IL-25, RCAS1, RS5, RAGE, RANTES, Ras, T101, SAGE, SAP-1, 5100, SSX-2, survivin, survivin-2B, SDDCAG16, TA-90\Mac2 binding protein, TAAL6, TAC, TAG-72, TGF-βRII, Ig TCR, TLP, telomerase, tenascin, TRAIL receptors, TRP-1, TRP-2, TSP-180, TNF-α, Tn antigen, Thomson-Friedenreich antigens, tumor necrosis antigens, tyrosinase, VEGFR, ED-B fibronectin, WT-1, XAGE, 17-1A-antigen, complement factors C3, C3a, C3b, C5a, C5, an angiogenesis marker, bc1-2, bc1-6, and K-ras, an oncogene marker and an oncogene product. In some cases, the endogenous antigen presented by the cancer cell line may not be well studied or unknown. The identity or sequence of the endogenous antigen can be determined, for example, by sequencing.

### Cancer Cell Lines

The cancer cell lines described herein can be mammalian cancer cell lines (e.g., human cancer cell lines). The cancer cell lines can be derived from a sample obtained from a human subject having a tumor. The sample can be various samples described herein. The sample can be a liquid sample or a solid tissue sample. For example, the sample can be a tissue from brain, liver, lung, kidney, prostate, ovary, spleen, lymph node (e.g., tonsil), thyroid, thymus, pancreas, heart, skeletal muscle, intestine, larynx, esophagus, or stomach. Additional non-limiting sources include bone marrow, cord blood, tissue from a site of infection, ascites, pleural effusion, spleen tissue, and tumors. As described herein, the antigen-presenting cell (APC) used for TCR identification in various embodiments can be the cancer cell line.

The cancer cell lines can be engineered or personalized to exogenously express one or more MHC molecules derived from a subject such as a cancer patient. These MHC molecules can be referred to as subject-derived or subject-specific MHC molecules. For example, the cancer cell lines can exogenously express a MHC class I molecule, a MHC class II molecule, or a combination thereof, derived from the subject (e.g., the same subject from which the TCRs are obtained). The MHC class I molecule can comprise HLA-A, HLA-B, HLA-C, or any combination thereof. The MHC class II molecule can comprise HLA-DP, HLA-DM, HLA-DOA, HLA-DOB, HLA-DQ, HLA-DR, or any combination thereof. The cancer cell line can exogenously express at least one, two, three, four, five, six, seven, eight, nine, ten or more different MHC molecules (e.g., MHC class I, MHC class II, or a combination thereof). The cancer cell line can exogenously express a subset of or all MHC molecules derived from a subject or identified in a subject. The exogenous MHC molecule can comprise an MHC-I alpha derived from the subject and an endogenous B2M. The exogenous MHC molecule can comprise both an MHC-I alpha and a B2M derived from the subject. The exogenous MHC molecule can be a fusion protein of the MHC-I alpha and the B2M (B2M-MHC-I-alpha fusion). Optionally, the expression of the cell line's endogenous MHCs can be reduced or abolished to reduce the chance of T cell or TCR activation due to alloreactivity. A cancer cell line with reduced or abolished level of endogenous Class I and/or Class II MHC expression may be called an MHC-null (or HLA-null) cancer cell line. If a cancer cell line (whether or not it is MHC-null) expresses one or more exogenous MHC genes (e.g., B2M-MHC-I-alpha fusions), it can be called an MHC-engineered cancer cell line. If the MHC-engineered cancer cell line expresses one or more MHC genes derived from a subject (e.g., a patient or a subject having a condition such as cancer), it can be called an MHC-personalized cancer cell line. The MHC-personalized cancer cell line can express at least about one, two, three, four, five, six, seven, eight, nine, ten or more MHC genes derived from a subject. A polynucleotide or a sequence encoding the exogenous MHC molecule can be delivered into the cancer cell line for example. Various delivering methods or various vectors described in the present disclosure can be used. For example, the delivering methods or vectors used for constructing TCR-expressing vectors can be used to construct vectors comprising the sequence encoding the MHC molecule. For example, the vector can be a plasmid, a transposon (e.g., Sleeping Beauty, Piggy Bac), or a viral vector (e.g., adenoviral vector, AAV vector, retroviral vector and lentiviral vector). The exogenous MHC molecules can be transiently or stably expressed in the cancer cell line. In some cases, the polynucleotide encoding the exogenous MHC molecule can be delivered into the cancer cell line by electroporation. In some cases, the polynucleotide can be delivered into the cancer cell line by a carrier such as a cationic polymer. The polynucleotide can be DNA or RNA. For example, RNA such as mRNA sequence encoding an exogenous MHC molecule can be delivered into a host cell by electroporation. The exogenous MHC molecule can be expressed from a vector such as plasmid, transposon (e.g., Sleeping Beauty, Piggy Bac), and a viral vector (e.g., adenoviral vector, AAV vector, retroviral vector and lentiviral vector). Additional examples of a vector include a shuttle vector, a phagemide, a cosmid and an expression vector. Non-limiting examples of plasmid vectors include pUC, pBR322, pET, pBluescript, and variants thereof. Further, a vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In some cases, a mixture of two or more polynucleotides or sequences encoding two or more MHC genes derived from a subject can be delivered (e.g., electroporated, transfected, or transduced) into the cancer cell line. In some cases, the vector is a self-amplifying RNA replicon.

The cancer cell line can express an endogenous antigen. The endogenous antigen can be a tumor antigen described herein, e.g., a tumor-associated antigen or a tumor-specific antigen. The cancer cell line may not express an exogenous antigen or may not present an exogenous antigen. The endogenous antigen can be expressed from an endogenous polynucleotide of the cancer cell line. The endogenous antigen can be a protein product from an endogenous mRNA, which is transcribed from the genome of the cancer cell line.

The cancer cell lines described herein can be derived from various tissues. The cancer cell lines can be derived from various cancer or tumor types, including but not limited to, bladder cancer, bone cancer, brain cancer, breast cancer, colon cancer, ovarian cancer, head/neck cancer, leukemia, lymphoma, liver cancer, lung cancer, melanoma, pancreatic cancer, soft-tissue sarcoma, and stomach cancer. For example, the cancer cell line can be derived from adrenocortical carcinoma (ACC), bladder urothelial carcinoma (BLCA), breast invasive carcinoma (BRCA), cervical squamous cell carcinoma and endocervical adenocarcinoma (CESC), cholangiocarcinoma (CHOL), colon adenocarcinoma (COAD), rectum adenocarcinoma (READ), colorectal adenocarcinoma (COADREAD), lymphoid neoplasm diffuse large B-cell lymphoma (DLBC), esophageal carcinoma (ESCA), glioblastoma multiforme (GBM), brain lower grade glioma (LGG), head and neck squamous cell carcinoma (HNSC), kidney renal clear cell carcinoma (KIRC), kidney renal papillary cell carcinoma (KIRP), acute myeloid leukemia (LAML), chronic myelogenous leukemia (LCML), liver hepatocellular carcinoma (LIHC), lung adenocarcinoma (LUAD), lung squamous cell carcinoma (LUSC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), sarcoma (SARC), skin cutaneous melanoma (SKCM), stomach adenocarcinoma (STAD), testicular germ cell tumors (TGCT), thymoma (THYM), thyroid carcinoma (THCA), uterine carcinosarcoma (UCS), uterine corpus endometrial carcinoma (UCEC), or uveal melanoma (UVM).

The cancer cell line can be various types of cell lines. The cancer cell line can be selected based on which cancer a subject has. The selected cancer cell line can then be used to screen antigen-reactive T cells or TCRs as described herein. The cancer cell line can be derived from BLCA. For example, the cancer cell line can be RT4, CAL29, RT112, SW780, or KMBA2. The cancer cell line can be derived from BRCA. For example, the cancer cell line can be BT483, HCC1500, ZR7530, HCC38, or HCC1143. The cancer cell line can be derived from CHOL. For example, the cancer cell line can be SNU1079, SNU478, SNU869, SNU245, or HUCCT1. The cancer cell line can be derived from COADREAD. For example, the cancer cell line can be SW837, CL34, HCC56, HT55, or LS411N. The cancer cell line can be derived from DLBC. For example, the cancer cell line can be CI1, RI1, DOHH2, WSUDLCL2, or SUDHL6. The cancer cell line can be derived from ESCA. For example, the cancer cell line can be OE21, TE11, TE9, OE19, or OE33. The cancer cell line can be derived from GBM. For example, the cancer cell line can be SNU201, SNU626, CAS1, SNU489, or YKG1. The cancer cell line can be derived from HNSC. For example, the cancer cell line can be SCC15, BICR16, SNU1214, SCC25, or BICR31. The cancer cell line can be derived from KIRC. For example, the cancer cell line can be KMRC20, KMRC3, VMRCRCZ, CAL54, or RCC10RGB. The cancer cell line can be derived from LAML. For example, the cancer cell line can be KASUMI6, KG1, GDM1, OCIAML5, or MEI. The cancer cell line can be derived from LGG. For example, the cancer cell line can be H4, NMCG1, TM31, SW1088, or HS683. The cancer cell line can be derived from LIHC. For example, the cancer cell line can be HEPG2, JHH5, HUH7, HUH1, or HEP3B217. The cancer cell line can be derived from LUAD. For example, the cancer cell line can be NCIH3255, HCC2935, NCIH1734, RERFLCAD1, or HCC4006. The cancer cell line can be derived from LUSC. For example, the cancer cell line can be SW900, NCIH2170, HCC95, LUDLU1, or KNS62. The cancer cell line can be derived from MESO. For example, the cancer cell line can be ISTMES2, JL1, ISTMES1, NCIH2452, or MPP89. The cancer cell line can be derived from OV. For example, the cancer cell line can be CAOV4, KURAMOCHI, COV362, OVSAHO, or JHOS4. The cancer cell line can be derived from PAAD. For example, the cancer cell line can be PATU8988S, CAPAN1, TCCPAN2, PANC0504, or PANC0327. The cancer cell line can be derived from PRAD. For example, the cancer cell line can be VCAP, MDAPCA2B, LNCAPCLONEFGC, DU145, or 22RV1. The cancer cell line can be derived from SKCM. For example, the cancer cell line can be HS939T, MALME3M, UACC257, HS944T, or RPMI7951. The cancer cell line can be derived from STAD. For example, the cancer cell line can be HUG1N, SNU620, SNU16, SNU601, or GSU. The cancer cell line can be derived from THCA. For example, the cancer cell line can be ML1, BCPAP, FTC133, 8305C, or 8505C. The cancer cell line can be derived from UCEC. For example, the cancer cell line can be MFE280, KLE, RL952, JHUEM3, or EFE184.

Additional examples of cancer cell lines described herein include, but are not limited to, 253J, 253JBV, 5637, 639V, 647V, BC3C, BFTC905, CAL29, HS172T, HT1197, HT1376, J82, JMSU1, KMBC2, KU1919, RT112, RT4, SCABER, SW1710, SW780, T24, TCCSUP, UBLC1, UMUC1, UMUC3, VMCUB1 or other types of cancer cell lines derived from BLCA; AU565, BT20, BT474, BT483, BT549, CAL120, CAL148, CAL51, CAL851, CAMA1, DU4475, EFM19, EFM192A, HCC1143, HCC1187, HCC1395, HCC1419, HCC1428, HCC1500, HCC1569, HCC1599, HCC1806, HCC1937, HCC1954, HCC202, HCC2157, HCC2218, HCC38, HCC70, HDQP1, HMC18, HS281T, HS343T, HS578T, HS606T, HS739T, HS742T, JIMT1, KPL1, MCF7, MDAMB134VI, MDAMB157, MDAMB175VII, MDAMB231, MDAMB361, MDAMB415, MDAMB436, MDAMB453, MDAMB468, SKBR3, T47D, UACC812, UACC893, ZR751, ZR7530 or other types of cancer cell lines derived from BRCA; HUCCT1, SNU1079, SNU1196, SNU245, SNU308, SNU478, SNU869 or other types of cancer cell lines derived from CHOL; C2BBE1, CCK81, CL11, CL14, CL34, CL40, COL0201, COL0320, COL0678, CW2, GP2D, HCC56, HCT116, HCT15, HS255T, HS675T, HS698T, HT115, HT29, HT55, KM12, LOVO, LS1034, LS123, LS180, LS411N, LS513, MDST8, NCIH508, NCIH716, NCIH747, OUMS23, RCM1, RKO, SKCO1, SNU1033, SNU1040, SNU1197, SNU175, SNU407, SNU503, SNU61, SNU81, SNUC1, SNUC2A, SNUC4, SNUC5, SW1116, SW1417, SW1463, SW403, SW48, SW480, SW620, SW837, SW948, T84 or other types of cancer cell lines derived from COADREAD; A3KAW, A4FUK, CI1, DB, DOHH2, HT, KARPAS422, MC116, NUDHL1, NUDUL1, OCILY19, PFEIFFER, RI1, RL, SUDHL10, SUDHL4, SUDHL5, SUDHL6, SUDHL8, TOLEDO, U937, WSUDLCL2 or other types of cancer cell lines derived from DLBC; COL0680N, ECGI10, KYSE140, KYSE150, KYSE180, KYSE270, KYSE30, KYSE410, KYSE450, KYSE510, KYSE520, KYSE70, OE19, OE21, OE33, TE1, TE10, TE11, TE14, TE15, TE4, TE5, TE6, TE8, TE9 or other types of cancer cell lines derived from ESCA; 42MGBA, 8MGBA, A172, AM38, CAS1, CCFSTTG1, DBTRGOSMG, DKMG, GAMG, GB1, GMS10, GOS3, KALS1, KG1C, KNS42, KNS60, KNS81, KS1, LN18, LN229, M059K, SF126, SF295, SNU1105, SNU201, SNU466, SNU489, SNU626, T98G, U118MG, U251MG, U87MG, YH13, YKG1 or other types of cancer cell lines derived from GBM; A253, BHY, BICR16, BICR18, BICR22, BICR31, BICR56, BICR6, CAL27, CAL33, FADU, HS840T, HSC2, HSC3, HSC4, PECAPJ15, PECAPJ49, SCC15, SCC25, SCC4, SCC9, SNU1041, SNU1066, SNU1076, SNU1214, SNU899, YD10B, YD15, YD38, YD8 or other types of cancer cell lines derived from HNSC; 769P, 7860, A498, A704, ACHN, CAKI1, CAKI2, CAL54, KMRC1, KMRC2, KMRC20, KMRC3, OSRC2, RCC10RGB, SNU1272, SNU349, VMRCRCZ or other cancer cell lines derived from KIRC; AML193, EOL1, F36P, GDM1, HEL, HEL9217, HL60, KASUMI1, KASUMI6, KG1, MO7E, MEI, MOLM13, MOLM16, MONOMAC1, MONOMAC6, MV411, NB4, NOMO1, OCIAML2, OCIAML3, OCIAML5, OCIM1, P31FUJ, PL21, SIGM5, SKM1, TF1, THP1 or other types of cancer cell lines derived from LAML; GI1, H4, HS683, NMCG1, SNU738, SW1088, SW1783, TM31 or other types of cancer cell lines derived from LGG; HEP3B217, HEPG2, HLF, HUH1, HUH6, HUH7, JHH1, JHH2, JHH4, JHH5, JHH6, JHH7, LI7, NCIH684, PLCPRF5, SKHEP1, SNU182, SNU387, SNU398, SNU423, SNU449, SNU475, SNU761, SNU878, SNU886 or other types of cancer cell lines derived from LIHC; A549, ABC1, CAL12T, CALU3, CORL105, DV90, HCC1171, HCC1833, HCC2108, HCC2279, HCC2935, HCC4006, HCC44, HCC78, HCC827, HS229T, HS618T, LU65, LXF289, MORCPR, NCIH1299, NCIH1355, NCIH1395, NCIH1435, NCIH1437, NCIH1563, NCIH1568, NCIH1573, NCIH1623, NCIH1648, NCIH1650, NCIH1651, NCIH1666, NCIH1693, NCIH1703, NCIH1734, NCIH1755, NCIH1781, NCIH1792, NCIH1793, NCIH1838, NCIH1944, NCIH1975, NCIH2009, NCIH2023, NCIH2030, NCIH2073, NCIH2085, NCIH2087, NCIH2106, NCIH2110, NCIH2122, NCIH2126, NCIH2172, NCIH2228, NCIH2291, NCIH23, NCIH2342, NCIH2347, NCIH2405, NCIH2444, NCIH322, NCIH3255, NCIH358, NCIH441, NCIH522, NCIH650, NCIH838, NCIH854, PC14, RERFLCAD1, RERFLCAD2, RERFLCKJ, RERFLCMS, SKLU1 or other types of cancer cell line derived from LUAD; CALU1, EBC1, EPLC272H, HARA, HCC15, HCC1588, HCC95, HLFA, KNS62, LC1F, LK2, LOUNH91, LUDLU1, NCIH1385, NCIH1869, NCIH2170, NCIH226, NCIH520, RERFLCAI, RERFLCSQ1, SKMES1, SQ1, SW1573, SW900 or other types of cancer cell lines derived from LUSC; ACCMESO1, DM3, ISTMES1, ISTMES2, JL1, MPP89, MSTO211H, NCIH2052, NCIH2452, NCIH28, RS5 or other types of cancer cell lines derived from MESO; 59M, A2780, CAOV3, CAOV4, COV318, COV362, COV644, EFO21, EF027, ES2, FUOV1, HEYA8, IGROV1, JH005, JHOM1, JHOM2B, JHOS2, JHOS4, KURAMOCHI, MCAS, OAW28, OAW42, OC314, ONCODG1, OV56, OV7, OV90, OVCAR4, OVCAR8, OVISE, OVK18, OVMANA, OVSAHO, OVTOKO, RMGI, RMUGS, SKOV3, SNU119, SNUB, SNU840, TOV112D, TOV21G, TYKNU or other types of cancer cell lines derived from OV; ASPC1, BXPC3, CAPAN1, CAPAN2, CFPAC1, DANG, HPAC, HPAFII, HS766T, HUPT3, HUPT4, KP2, KP3, KP4, L33, MIAPACA2, PANC0203, PANC0213, PANC0327, PANC0403, PANC0504, PANC0813, PANC1, PANC1005, PATU8902, PATU8988S, PATU8988T, PK1, PK45H, PK59, PSN1, QGP1, SNU213, SNU324, SNU410, SU8686, SUIT2, SW1990, T3M4, TCCPAN2, YAPC or other types of cancer cell lines derived from PAAD; 22RV1, DU145, LNCAPCLONEFGC, MDAPCA2B, NCIH660, PC3, VCAP or other types of cancer cell lines derived from PRAD; A101D, A2058, A375, C32, CJM, COL0679, COL0741, COL0783, COL0792, COLO800, COL0829, G361, HMCB, HS294T, HS600T, HS695T, HS834T, HS839T, HS852T, HS895T, HS934T, HS936T, HS939T, HS940T, HS944T, HT144, IGR1, IGR37, IPC298, K029AX, LOXIMVI, MALME3M, MDAMB435S, MELHO, MELJUSO, MEWO, RPMI7951, SH4, SKMEL1, SKMEL24, SKMEL28, SKMEL3, SKMEL30, SKMEL31, SKMEL5, UACC257, UACC62, WM115, WM1799, WM2664, WM793, WM88, WM983B or other types of cancer cell lines derived from SKCM; 2313287, AGS, ECC10, ECC12, FU97, GCIY, GSS, GSU, HGC27, HS746T, HUG1N, HUTU80, IM95, KATOIII, KE39, KE97, LMSU, MKN1, MKN45, MKN7, MKN74, NCCSTCK140, NCIN87, NUGC2, NUGC3, NUGC4, OCUM1, RERFGC1B, SH10TC, SNU1, SNU16, SNU216, SNU5, SNU520, SNU601, SNU620, SNU668, SNU719, TGBC11TKB or other types of cancer cell lines derived from STAD; 8305C, 8505C, BCPAP, BHT101, CAL62, FTC133, FTC238, ML1, SW579, TT, TT2609CO₂ or other types of cancer cell lines derived from THCA; AN3CA, COL0684, EFE184, EN, ESS1, HEC108, HEC151, HEC1A, HEC1B, HEC251, HEC265, HEC50B, HEC59, HEC6, JHUEM1, JHUEM2, JHUEM3, JHUEM7, KLE, MFE280, MFE296, MFE319, RL952, SNGM, SNU1077, SNU685, TEN or other cancer cell lines derived from UCEC.

The cancer cell line described herein can be a mixture of two or more types of cancer cell lines. The cancer cell line described herein can be a mixture of two or more types of cancer cell lines derived from a same cancer or tumor type. The cancer cell line described herein can be a mixture of two or more types of cancer cell lines derived from different cancers or tumor types. The cancer cell line described herein can be a mixture of two or more types of cancer cell lines derived from a same tissue. The cancer cell line described herein can be a mixture of two or more types of cancer cell lines derived from different tissues. In some cases, one or more cancer cell lines can be chosen to carry out the methods described herein depending on the cancer or cancers a subject has.

In some aspects, the present disclosure also provides a composition for identifying an antigen-reactive cell that recognizes an endogenous antigen of a cancer cell line in complex with an MHC molecule expressed by a subject. The composition can comprise a cell that is a cancer cell line expressing an endogenous antigen in complex with an exogenous MHC molecule. The exogenous MHC molecule can be the MHC molecule expressed by the subject or derived from the subject. Optionally, the composition can further comprise a T cell expressing a natively paired TCR derived from the subject. A gene expression profile, a transcriptomic profile or a genomic alternation of the cancer cell line can resemble (e.g., be substantially similar with) that of a cancer cell from the subject. For example, a correlation coefficient of the gene expression profile, the transcriptomic profile or the genomic alteration between the cancer cell line and the primary cancer cell or the tumor sample can be equal to or greater than about 0.1, 0.2, 0.3, 0.4, 0.5, 0.6, 0.7, 0.8, 0.9 or more.

The cancer cell line may not comprise or present an exogenous antigen. In some cases, an endogenous MHC molecule of the cancer cell line can be inactivated (e.g., down regulated, knocked out or knocked down). To inactivate a gene or a protein product of a gene, the gene encoding the protein can be knocked out or knocked down. The cancer cell line can be null for an endogenous MHC molecule. The cancer cell line can be null for all endogenous MHC molecules. The endogenous MHC molecule can comprise a MHC class I molecule, a MHC class II molecule, or a combination thereof. The MHC class I molecule can comprise HLA-A, HLA-B, HLA-C, or any combination thereof. In some cases, an alpha chain of the MHC class I molecule (MHC-I alpha) can be inactivated. For example, a gene encoding the alpha chain of the MHC class I molecule can be inactivated. In some cases, a beta-2-microglobulin (B2M) of the MHC class I molecule can be inactivated. For example, a gene encoding the B2M of the MHC class I molecule can be inactivated. The MHC class II molecule can comprise HLA-DP, HLA-DM, HLA-DOA, HLA-DOB, HLA-DQ, HLA-DR, or any combination thereof. In some cases, an alpha chain or a beta chain of the MHC class II molecule can be inactivated. For example, a gene encoding the alpha chain or the beta chain of the MHC class II molecule can be inactivated. For example, a gene regulating transcription of the MHC class II molecule can be inactivated. The gene can be Class II major histocompatibility complex transactivator (CIITA). The exogenous MHC molecule of the cancer cell line can comprise a MHC class I molecule, a MHC class II molecule, or a combination thereof, derived from the subject. The MHC class I molecule can comprise HLA-A, HLA-B, HLA-C, or any combination thereof. The MHC class II molecule can comprise HLA-DP, HLA-DM, HLA-DOA, HLA-DOB, HLA-DQ, HLA-DR, or any combination thereof. The exogenous MHC molecule can comprise an MHC-I alpha derived from the subject and an endogenous B2M. The exogenous MHC molecule can comprise both an MHC-I alpha and a B2M derived from the subject. The exogenous MHC molecule can be a fusion protein of the MHC-I alpha and the B2M (B2M-MHC-I-alpha fusion). The MHC-I alpha and the B2M can be linked by a linker. The linker can be (G₄S)ₙ, wherein G is glycine, S is serine, and n can be any integer from 1 to 10. The exogenous MHC molecule can comprise an MHC-II alpha and an MHC-II beta derived from the subject. The T cell can comprise a plurality of T cells, each expressing a different natively paired TCR derived from the subject. The plurality of T cells can comprise at least 10 different natively paired TCRs derived from the subject.

In some aspects, the present disclosure also provides a composition comprising a panel of MHC-engineered cancer cell lines derived from a same cancer type. For example, the panel of MHC-engineered cancer cell lines can be derived from bladder cancer, bone cancer, brain cancer, breast cancer, colon cancer, ovarian cancer, head/neck cancer, leukemia, lymphoma, liver cancer, lung cancer, melanoma, pancreatic cancer, soft-tissue sarcoma, or stomach cancer. The panel can comprise a first sub-panel comprising at least two MHC-engineered cancer cell lines derived from a same first parental cancer cell line. As described herein, if a first cell that is a cancer cell line is engineered (e.g., by exogenously expressing an MHC molecule) to form the second cell, then the first cell can be called the parental cell line. The parental cell line can be the original cell line which has not been engineered with subject-specific HLA(s). The panel can comprise a second sub-panel comprising at least two MHC-engineered cancer cell lines derived from a same second parental cancer cell line. The at least two MHC-engineered cancer cell lines of the first sub-panel or the second sub-panel can express a different exogenous MHC molecule. The at least two MHC-engineered cancer cell lines of the first sub-panel or the second sub-panel may not express a same exogenous and/or endogenous MHC molecule. The at least two MHC-engineered cancer cell lines may comprise at least about 10, 20, 30, 40, 50, 60, 70, 80, 90, 100, or more MHC-engineered cancer cell lines, each MHC-engineered cancer cell line expressing a different exogenous MHC molecule. For example, each two of them may not express a same exogenous and/or endogenous MHC molecule.

The first parental cancer cell line and the second parental cancer cell line can be different. The endogenous MHC molecule of the at least two MHC-engineered cancer cell lines of the first sub-panel or the second sub-panel can be inactivated. The exogenous MHC molecule can be expressed by a subject or derived from the subject. The panel can comprise three or more sub-panels. In each sub-panel, there may be three or more MHC-engineered cancer cell lines derived from a same parental cancer cell line, each expressing a different exogenous MHC molecule.

As a non-limiting example, the panel of MHC-engineered cancer cell lines can be derived from colorectal. The first sub-panel can comprise MHC-engineered cancer cell lines derived from parental cancer cell line SW837. The second sub-panel can comprise MHC-engineered cancer cell lines derived from parental cancer cell line HT55. A patient of colon cancer may have the following Class I MHC genes: HLA-A*02:01, HLA-A*24:02, HLA-B*39:05, HLA-B*51:01, HLA-C*07:02, and HLA-C*15:02. In the first sub-panel, one cell may be engineered to express HLA-A*02:01, and another cell may be engineered to express any of the above Class I MHC genes except HLA-A*02:01 (e.g., HLA-B*39:05). In the second sub-panel, one cell may be engineered to express HLA-C*07:02, and another cell may be engineered to express any of the above Class I MHC genes except HLA-C*07:02 (e.g., HLA-A*24:02).

The composition can further comprise a plurality of T cells. Each cancer cell line of the at least two MHC-engineered cancer cell lines in the first sub-panel or the second sub-panel can be mixed (e.g., co-cultured) with the plurality of T cells. The plurality of T cells can comprise at least two different natively paired TCRs. The natively paired TCRs can be derived from the same subject.

### Gene Delivery

Various methods of delivering (or introducing) and expressing genes or genetic materials (e.g., nucleic acid molecules encoding proteins of interest) into a cell can be used. The proteins of interest described herein can be exogenous MHC molecules or TCR chains. In the context of an expression vector, the vector can be readily introduced into a host cell, e.g., APC, cancer cell line, or T cell. For example, the expression vector can be transferred into a host cell by physical, chemical, or biological methods. Physical methods for introducing a nucleic acid molecule into a host cell include, but are not limited to, calcium phosphate precipitation, lipofection, particle bombardment, microinjection, electroporation, and the like. Biological methods for introducing a nucleic acid molecule of interest into a host cell include, but are not limited to, the use of DNA and RNA vectors. Viral vectors such as retroviral vectors, lentiviral vectors, adenoviral vectors and adeno-associated viral vectors, can be used for delivering genes into mammalian cells, e.g., human cells. Chemical methods for introducing a nucleic acid molecule into a host cell can include colloidal dispersion systems, such as macromolecule complexes, nanocapsules, microspheres, beads, and lipid-based systems including oil-in-water emulsions, micelles, mixed micelles, and liposomes. An example colloidal system for use as a delivery vehicle in vitro and in vivo is a liposome (e.g., an artificial membrane vesicle). Other methods can include, but are not limited to, delivery of nucleic acids with targeted nanoparticles or other suitable sub-micron sized delivery system.

In the case where a non-viral delivery system is utilized, an example delivery vehicle is a liposome. The use of lipid formulations can be contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform may be used as the solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They can form spontaneously when phospholipids are suspended in an excess of aqueous solution. The lipid components can undergo self-rearrangement before the formation of closed structures and entrap water and dissolved solutes between the lipid bilayers. However, compositions that have different structures in solution than the normal vesicular structure are also encompassed. For example, the lipids may assume a micellar structure or merely exist as nonuniform aggregates of lipid molecules. Also contemplated herein can include lipofectamine-nucleic acid complexes.

### Gene Editing

The cancer cell line (or in some cases, the non-cancer cell) disclosed herein can be engineered to inactivate one or more endogenous MHC molecules. A gene encoding the endogenous MHC molecule or a subunit thereof can be inactivated using a gene editing technique such as clustered regularly interspaced short palindromic repeats (CRISPR®, see, e.g., U.S. Pat. No. 8,697,359), transcription activator-like effector (TALE) nucleases (TALENs, see, e.g., U.S. Pat. No. 9,393,257), meganucleases (endodeoxyribonucleases having large recognition sites comprising double-stranded DNA sequences of 12 to 40 base pairs), zinc finger nuclease (ZFN, see, e.g., Urnov et al., Nat. Rev. Genetics (2010) v11, 636-646), or megaTAL nucleases (a fusion protein of a meganuclease to TAL repeats) methods. Alternatively, a gene of interest described herein can be knocked down using techniques such as RNA interference (RNAi).

These gene-editing techniques may share a common mode of action in binding a user-defined sequence of DNA and mediating a double-stranded DNA break (DSB). DSB may then be repaired by either non-homologous end joining (NHEJ) or—when donor DNA is present—homologous recombination (HR), an event that introduces the homologous sequence from a donor DNA fragment. Additionally, nickase nucleases generate single-stranded DNA breaks (SSB). DSBs may be repaired by single strand DNA incorporation (ssDI) or single strand template repair (ssTR), an event that introduces the homologous sequence from a donor DNA.

Genetic modification of genomic DNA can be performed using site-specific, rare-cutting endonucleases that are engineered to recognize DNA sequences in the locus of interest. Methods for producing engineered, site-specific endonucleases are known in the art. For example, zinc-finger nucleases (ZFNs) can be engineered to recognize and cut predetermined sites in a genome. ZFNs are chimeric proteins comprising a zinc finger DNA-binding domain fused to the nuclease domain of the Fokl restriction enzyme. The zinc finger domain can be redesigned through rational or experimental methods to produce a protein that binds to a pre-determined DNA sequence (e.g., sequence with 18 basepairs in length). By fusing this engineered protein domain to the Fokl nuclease, it is possible to target DNA breaks with genome-level specificity. ZFNs can be used to target gene addition, removal, and substitution in a wide range of eukaryotic organisms. Likewise, TAL-effector nucleases (TALENs) can be generated to cleave specific sites in genomic DNA. Like a ZFN, a TALEN comprises an engineered, site-specific DNA-binding domain fused to the Fokl nuclease domain. In this case, however, the DNA binding domain comprises a tandem array of TAL-effector domains, each of which specifically recognizes a single DNA basepair. Compact TALENs have an alternative endonuclease architecture that avoids the need for dimerization. A Compact TALEN can comprise an engineered, site-specific TAL-effector DNA-binding domain fused to the nuclease domain from the I-Teel homing endonuclease. Unlike Fokl, I-Teel may not dimerize to produce a double-strand DNA break so a Compact TALEN can function as a monomer.

Engineered endonucleases based on the CRISPR/Cas9 system can also be used. The CRISPR gene-editing technology can comprise an endonuclease protein whose DNA-targeting specificity and cutting activity can be programmed by a short guide RNA or a duplex crRNA/TracrRNA. A CRISPR endonuclease comprises two components: (1) a caspase effector nuclease, typically microbial Cas9; and (2) a short “guide RNA” or a RNA duplex comprising a 18 to 20 nucleotide targeting sequence that directs the nuclease to a location of interest in the genome. By expressing multiple guide RNAs in the same cell, each having a different targeting sequence, it is possible to target DNA breaks simultaneously to multiple sites in the genome (multiplex genomic editing).

There are two classes of CRISPR systems, each containing multiple CRISPR types. Class I contains type I and type III CRISPR systems that are commonly found in Archaea. And, Class II contains type II, IV, V, and VI CRISPR systems. Although the most widely used CRISPR/Cas system is the type II CRISPR-Cas9 system, CRISPR/Cas systems have been repurposed for genome editing. More than 10 different CRISPR/Cas proteins have been remodeled within last few years. For example, Cas12a (Cpf1) proteins from Acid-aminococcus sp (AsCpf1) or Lachnospiraceae bacterium (LbCpf1) can be used.

Homing endonucleases are a group of naturally occurring nucleases that recognize 15-40 base-pair cleavage sites commonly found in the genomes of plants and fungi. They can be associated with parasitic DNA elements, such as group 1 self-splicing introns and inteins. They can naturally promote homologous recombination or gene insertion at specific locations in the host genome by producing a double-stranded break in the chromosome, which recruits the cellular DNA-repair machinery. Specific amino acid substations can reprogram DNA cleavage specificity of homing nucleases. Meganucleases (MN) are monomeric proteins with innate nuclease activity that are derived from bacterial homing endonucleases and engineered for a unique target site. In some cases, meganuclease is engineered I-CreI homing endonuclease. In other cases, meganuclease is engineered I-SceI homing endonuclease.

In addition to above mentioned gene editing technologies, chimeric proteins comprising fusions of meganucleases, ZFNs, and TALENs can be engineered to generate novel monomeric enzymes that take advantage of the binding affinity of ZFNs and TALENs and the cleavage specificity of meganucleases. For example, megaTAL is a single chimeric protein, which is the combination of the easy-to-tailor DNA binding domains from TALENs with the high cleavage efficiency of meganucleases.

In order to perform the gene editing technique, the nucleases, and in the case of the CRISPR/Cas9 system, a gRNA, can be delivered to the cells of interest. Delivery methods include but are not limited to physical, chemical, and viral methods. In some instances, physical delivery methods can be selected from the methods including but not limited to electroporation, microinjection, or use of ballistic particles. On the other hand, chemical delivery methods may use molecules such calcium phosphate, lipid, or protein. In some embodiments, viral delivery methods can use viruses such as adenovirus, lentivirus, or retrovirus.

### Pharmaceutical Compositions

The present disclosure also provides pharmaceutical compositions comprising an antigen-reactive cell, a TCR identified by the methods described herein, or a cell expressing a TCR identified by the methods described herein in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions of the present disclosure can be formulated for intravenous administration.

Also provided herein is a composition for use as a medicament, the composition comprising an antigen-reactive cell, a TCR identified by the methods described herein, or a cell expressing a TCR identified by the methods described herein. The composition can be a pharmaceutical composition comprising one or more pharmaceutically or physiologically acceptable carriers, diluents or excipients. The composition can be used to treat a disease such as cancer or autoimmune disease.

Pharmaceutical compositions of the present disclosure may be administered in a manner appropriate to the disease to be treated (or prevented). The quantity and frequency of administration can be determined by such factors as the condition of the patient, and the type and severity of the patient's disease, although appropriate dosages may be determined by clinical trials.

The pharmaceutical composition can be substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some cases, the bacterium can be at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, Streptococcus pyogenes group A, and any combinations thereof.

### Administration

Provided herein can be methods for administering a pharmaceutical composition or a therapeutic regime to a subject having a condition such as cancer. The pharmaceutical composition can be a cellular composition comprising an antigen-reactive cell, a TCR, or a cell expressing a TCR identified by the methods described herein. The pharmaceutical composition can be a solution comprising a drug that is an antigen-reactive cell, a TCR, or a cell expressing a TCR identified by the methods described herein. In some instances, the cellular composition can be provided in a unit dosage form. The cellular composition can be resuspended in solution and administered as an infusion. Provided herein can also be a treatment regime that includes immunostimulants, immunosuppressants, antibiotics, antifungals, antiemetics, chemotherapeutics, radiotherapy, and any combination thereof. A treatment regime that includes any of the above can be lyophilized and reconstituted in an aqueous solution (e.g., saline solution). In some instances, a treatment (for example, a cellular treatment) is administered by a route selected from subcutaneous injection, intramuscular injection, intradermal injection, percutaneous administration, intravenous (“i.v.”) administration, intranasal administration, intralymphatic injection, and oral administration. In some instances, a subject is infused with a cellular composition comprising immunoreceptor-programmed recipient cells by an intralymphatic microcatheter.

For a subcutaneous route, a needle may be inserted into fatty tissue just beneath the skin. After a drug is injected, it can move into small blood vessels (capillaries) and can be carried away by the bloodstream. Alternatively, a pharmaceutical composition can reach the bloodstream through the lymphatic vessels. The intramuscular route may be used when larger volumes of the pharmaceutical composition are needed. Because the muscles lie below the skin and fatty tissues, a longer needle may be used. A pharmaceutical composition can be injected into the muscle of the upper arm, thigh, or buttock. For the intravenous route, a needle can be inserted directly into a vein. The pharmaceutical composition can be a solution containing the cells and may be given in a single dose or by continuous infusion. For infusion, the solution can be moved by gravity (from a collapsible plastic bag) or, more commonly, by an infusion pump through thin flexible tubing to a tube (catheter) inserted in a vein, usually in the forearm. In some cases, cells or therapeutic regimes are administered as infusions. An infusion can take place over a period of time. For example, an infusion can be an administration of a cell or therapeutic regime over a period of about 5 minutes to about 5 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, or up to about 5 hours.

In some embodiments, intravenous administration is used to deliver a precise dose quickly and in a well-controlled manner throughout the body. It can also be used for irritating solutions, which would cause pain and damage tissues if given by subcutaneous or intramuscular injection. An intravenous injection may be more difficult to administer than a subcutaneous or intramuscular injection because inserting a needle or catheter into a vein may be difficult, especially if the person is obese. When given intravenously, a drug can be delivered immediately to the bloodstream and tend to take effect more quickly than when given by any other route. Consequently, health care practitioners can closely monitor people who receive an intravenous injection for signs that the drug is working or is causing undesired side effects. Also, the effect of a drug given by this route may tend to last for a shorter time. Therefore, some drugs can be given by continuous infusion to keep their effect constant. For the intrathecal route, a needle can be inserted between two vertebrae in the lower spine and into the space around the spinal cord. The drug can then be injected into the spinal canal. A small amount of local anesthetic can be used to numb the injection site. This route can be used when a drug is needed to produce rapid or local effects on the brain, spinal cord, or the layers of tissue covering them (meninges)—for example, to treat infections of these structures.

In some cases, a pharmaceutical composition comprising a cellular therapy can be administered either alone or together with a pharmaceutically acceptable carrier or excipient, by any routes, and such administration can be carried out in both single and multiple dosages. More particularly, the pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes.

In some cases, a therapeutic regime can be administered along with a carrier or excipient. Examples of carriers and excipients can include dextrose, sodium chloride, sucrose, lactose, cellulose, xylitol, sorbitol, malitol, gelatin, PEG, PVP, and any combination thereof. In some cases, an excipient such as dextrose or sodium chloride can be at a percent from about 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or up to about 15%. In some cases, a method of treating a disease in a subject may comprise transplanting to the subject one or more cells (including organs and/or tissues) comprising engineered cells such as cells exogenously expressing a TCR identified by the methods described herein. Cells prepared by intracellular genomic transplant can be used to treat cancer.

### EXAMPLES

### Example 1: MHC-Personalization of Cancer Cell Line

To abolish the endogenous Class I MEW, the expression of B2M can be knocked out or knocked down. Alternatively, the expression of the alpha chain of Class I MEW (MHC-I alpha) genes such as HLA-A, HLA-B and HLA-C can be knocked out or knocked down. To knock out the aforementioned genes, any gene editing tool such as ZFN, TALEN, CRISPR/Cas9, or their variants can be used. For example, Cas9 and the guide RNA (gRNA) targeting sequences 5′-ACTCACGCTGGATAGCCTCC-3′, 5′-GAGTAGCGCGAGCACAGCTA-3′, 5′-CAGTAAGTCAACTTCAATGT-3′ can be used to knock out B2M.

As another example, Cas9 and the gRNAs targeting sequences 5′-GCCGCCTCCCACTTGCGCT-3′ and 5′-CACATGCAGCCCACGAGCCG-3′, which flank the HLA-A gene can be used to cause the deletion of HLA-A. Using Cas9 and gRNAs targeting upstream sequence of HLA-B and downstream sequence of HLA-C may cause deletion of the HLA-B and HLA-C, which are adjacent to each other. gRNA targeting upstream sequence of HLA-B may target the sequences 5′-ATCCCTAAATATGGTGTCCC-3′ or 5′-TCCCTAAATATGGTGTCCCT-3′. gRNA targeting downstream sequence of HLA-C may target the sequences 5′-GTGATCCGGGTATGGGCAGT-3′ or 5′-TGATCCGGGTATGGGCAGTG-3′. Together, these manipulations may cause the knock-out of MHC-I-alpha genes.

After the knock-out or knock-down of MHC-I is performed, the cells may be stained with anti-MHC-I antibody and the cells with no or low level of MHC-I expressed may be isolated. Afterwards, optionally, monoclonal cell line starting from a single cell may be established.

When the MHC-I alpha genes are knocked out or knocked down, exogenous MHC-I alpha genes can be introduced by any vector such as plasmids, viral vectors, or mRNA. The translation product of the exogenous MHC-I alpha can complex with endogenous B2M.

When the B2M is knocked out or knocked down, MHC-I alpha may be introduced into the patient. In some cases, a fusion protein of B2M and MHC-I alpha (hereby called B2M-MHC-I-alpha fusion) may be introduced into the patient, where the MHC-I alpha is derived from the patient. A linker can be introduced between B2M and MHC-I alpha to facilitate proper folding. The following sequence is an example of such B2M-MHC-I-alpha fusion where the MHC-I-alpha is HLA-A*02:01:

MSRSVALAVLALLSLSGLEAIQRTPKIQVYSRHPAENGKSNFLNCYVSGF
HPSDIEVDLLKNGERIEKVEHSDLSFSKDWSFYLLYYTEFTPTEKDEYAC
RVNHVTLSQPKIVKWDRDMGGGGSGGGGSGGGGSGSHSMRYFFTSVSRPG
RGEPRFIAVGYVDDTQFVRFDSDAASQRMEPRAPWIEQEGPEYWDGETRK
VKAHSQTHRVDLGTLRGYYNQSEAGSHTVQRMYGCDVGSDWRFLRGYHQY
AYDGKDYIALKEDLRSWTAADMAAQTTKHKWEAAHVAEQLRAYLEGTCVE
WLRRYLENGKETLQRTDAPKTHMTHHAVSDHEATLRCWALSFYPAEITLT
WQRDGEDQTQDTELVETRPAGDGTFQKWAAVVVPSGQEQRYTCHVQHEGL
PKPLTLRWEPSSQPTIPIVGIIAGLVLFGAVITGAVVAAVMWRRKSSDRK
GGSYSQAASSDSAQGSDVSLTACKV

Patient-derived Class II MHC (MHC-II) may also be exogenously expressed in cancer cell line. Patient-derived MHC-II alpha and MHC-II beta can be both exogenously expressed in the cancer cell line using a variety of vectors such as plasmids, viral vectors, and mRNA. Similar to the concept above, endogenous MHC-II expression can be reduced or abolished. Note that although MHC-II can be expressed by professional APCs such as dendritic cell and macrophage, they may also be expressed by cancer cells, especially when the cancer cells are contacted by INF-gamma. To abolish endogenous MHC-II expression, all MHC-II genes can be knocked out, or CIITA, the master regulator of MHC-II and its related genes, can be knocked out. CIITA can be knocked out with Cas9 and gRNA targeting the following sequence: 5′-TCCATCTGGTCATAGAAG-3′. Other MHC-II genes such as invariant chain and HLA-DM may also be exogenously expressed in the cancer cell line.

A cancer cell line with reduced level of endogenous Class I and/or Class II MHC expression may be called an MHC-null (or HLA-null) cancer cell line. If a cancer cell line (whether or not it is MHC-null) expresses one or more exogenous MHC genes (including B2M-MHC-I-alpha fusions), it can be called an MHC-engineered cancer cell line. If the MHC-engineered cancer cell line expresses one or more MHC genes that a patient has, it can be called an MHC-personalized cancer cell line.

### Example 2: MHC-Personalization of Non-Cancer Cells

The method described in Example 1 to produce MHC-null cancer cell lines can also be used to produce MHC-null stem cells such as induced pluripotent stem cells (iPSCs). The one or more MHC genes (including B2M-MHC-I-alpha fusion genes) can be stably introduced to iPSCs to via plasmid (via genomic integration), lentiviral vector, or CRISPR knock-in to produce MHC-engineered iPSCs. These MHC-null and MHC-engineered iPSCs can be artificially differentiated into a wide array of cell types (such as lung, liver, neural, pancreatic, heart, immune, hematopoietic stem cells, etc.) that can be considered non-cancer cells. These iPSC-derived non-cancer cells can be further engineered with exogenous MHC genes using the methods described above. Note that while it may be advantageous to stably express exogenous MHC in iPSC, transiently expressing exogenous MHC in iPSC-derived cells is a viable option. Transient expression can be achieved by plasmid, mRNA and AAV vectors. Immortalized primary human cells (e.g., via over-expression of SV40) may also function as non-cancer cells and can be MHC-engineered in the same way as iPSCs and cancer cell lines. If an MHC-engineered non-cancer cell expresses one or more MHC genes of a patient, it can be called an MHC-personalized non-cancer cell.

### Example 3: Discover Patient-Derived Tumor-Reactive TCRs Using MHC-Personalized Cancer Cells

As a non-limiting example, a patient of colon cancer may have the following Class I MHC genes: HLA-A*02:01, HLA-A*24:02, HLA-B*39:05, HLA-B*51:01, HLA-C*07:02, HLA-C*15:02, and the following Class II MHC genes: HLA-DPA1*02:02, HLA-DPB1*02:02, HLA-DPB1*19:01, HLA-DQA1*03:03, HLA-DQA1*01:03, HLA-DQB1*04:01, HLA-DQB1*06:01, HLA-DRA*01:01, HLA-DRB1*04:05, HLA-DRB1*08:03, HLA-DRB4*01:03.

To treat this patient, colorectal cancer cell lines: SW837, LS411N, HT55, CL34, SNU61 can be used. For each cell line, the B2M and CIITA can be knocked out to produce an MHC-null cell line. Then the mRNA encoding each of the B2M-MHC-I-alpha fusion genes and MHC-II genes can be prepared by standard in vitro transcription (IVT), capping and A-tailing. Equal concentration of mRNA of the 6 B2M-MHC-I-alpha fusion genes (HLA-A*02:01, HLA-A*24:02, HLA-B*39:05, HLA-B*51:01, HLA-C*07:02, HLA-C*15:02) can be mixed and electroporated into each MHC-null cell line to produce “MHC-I-personalized cancer cell lines”. Equal concentration of the mRNA of the 11 MHC-II genes (HLA-DPA1*02:02, HLA-DPB1*02:02, HLA-DPB1*19:01, HLA-DQA1*03:03, HLA-DQA1*01:03, HLA-DQB1*04:01, HLA-DQB1*06:01, HLA-DRA*01:01, HLA-DRB1*04:05, HLA-DRB1*08:03, HLA-DRB4*01:03) can be mixed and electroporated into each MHC-null cell line to produce “MHC-II-personalized cancer cell lines”. MHC-I-personalized cancer cell lines and MHC-II-personalized cancer cell lines can be collectively called MHC-personalized cell lines.

The tumor-infiltrating T cells or PD-1-high peripheral T cells can be prepared using standard method or the method described in Example 5. These cells may be subject to sequencing such as single-cell TCR-seq to obtain the paired TCR sequences for each T cell. A total of 1,000 to 10,000 paired TCR sequences may be obtained. All or a subset of these TCR genes may be synthesized in a pool using method described in International Application No. PCT/US2020/026558. The TCR genes may be introduced to peripheral T cells from healthy donor ex vivo using lentiviral vector, resulting in a population of T cells which we call “polyclonal synthetic T cells”. The exogenous (e.g., synthesized) TCRs may have murine constant domains to prevent mispairing between exogenous and endogenous TCRs in the polyclonal synthetic T cells.

To characterize the exogenous TCR gene pool, an aliquot of the polyclonal synthetic T cells can be obtained and the exogenous TCRs can be PCR-amplified using a pair of primers targeting the flanking sequencing of the exogenous TCR on the vector. And amplification product can be analyzed by NextGen Sequencing (NGS) and the frequency of each exogenous TCR in the pool can be recorded. These frequencies of exogenous TCRs in this sample can be called pre-selection frequencies.

The polyclonal synthetic T cells may be co-cultured with each of MHC-I-personalized or MHC-II-personalized cancer cell line, during which the synthetic T cell whose exogenous TCR recognizes the MHC-personalized cancer cell line may be activated. The activated T cells may express an activation marker such as CD137, CD69 and OX40. The activation marker-positive cells can be sorted using fluorescence activated cell sorting (FACS) or magnetic activated cell sorting (MACS). The exogenous TCR genes in the sorted cells can be PCR-amplified using a pair of primers targeting the flanking sequencing of the exogenous TCR on the vector. The amplification product can be analyzed by NGS and the frequency of each exogenous TCR in the pool can be recorded. These frequencies of exogenous TCRs in this sample can be called post-selection frequencies.

If an exogenous TCR's post-selection frequency is higher than its pre-selection frequency by a factor of 3 or more with 2 or more MHC-I-personalized or MHC-personalized cancer cell lines, this TCR can be regarded as a tumor-reactive TCR. Optionally, a control experiment can be performed where the MHC-personalized cancer cell line is replaced with MHC-null cancer cell line or MHC-personalized non-cancer cells whose organ or tissue origin is identical or similar to the MHC-personalized cancer cells. In this case the non-cancer cell may be MHC-personalized iPSC-derived colon cells or epithelial cell. If a TCR shows enrichment in the co-culture with MHC-personalized non-cancer cell, this TCR may be regarded self-reactive and deemed not suitable to be used in TCR-T therapy to the patient. If a TCR is tumor-reactive and not self-reactive, standard TCR-T manufacturing process can be applied to prepare TCR-T cells, which can be administered to the patient for cancer treatment.

### Example 4: Stimulate and Enrich Patient-Derived Tumor-Reactive T Cells

The MHC-personalized cancer cell lines can also be used to stimulate natural, patient-derived T cells (e.g., T cells without genetic manipulation). For example, tumor-infiltrating for peripheral tumor-experienced T cells can be isolated, enriched and optionally expanded using methods described in Example 5. These T cells can be co-cultured with MHC-personalized cancer cell line. The co-stimulation pathway can be induced during the co-culture process. For example, B7 molecules (CD80 and CD86) can be exogenous expressed in the MHC-personalized cancer cell lines. Alternatively, anti-CD28 antibody can be present in the culture media or on the surface of the cell culture vessel. As discussed before, in such co-cultures, the MHC-personalized cancer cell may be replaced by (1) autologous DCs fed with live or killed cancer cell line in the co-culture, (2) autologous DCs fed with live or killed autologous tumor cells, or (3) autologous tumor cells.

After 5 to 30 days of co-culture, the T cells can be collected, purified, subject to quality control (QC) assays and administered (e.g., infused) to the patient. To further increase the fraction of tumor-reactive T cells, after a short co-culture (e.g., 12 hours to 3 days), T cells with upregulated level of activation marker (e.g., CD69, CD25, CD137, OX40) can be isolated using FACS or MACS. These isolated cells may be further expanded (e.g., using the rapid expansion protocol or REP) before administration to the patient.

### Example 5: Obtaining Tumor-Infiltrating T Cells, Peripheral Tumor-Experienced T Cells and the Preparation of Tumor-Pulsed DCs

Obtaining tumor cells and TILs: If a patient of cancer undergoes tumor resection, a portion of the freshly resected tumor (with volume ideally greater than 1 cm 3) can be cryopreserved in liquid nitrogen. Optionally, another portion of the tumor material can be dissociated mechanically and/or enzymatically to small pieces and single-cell or near single-cell suspension. The single-cell or near single-cell suspension can be cryopreserved. The cryopreservation may be carried out in the presence of a cryoprotectant such as DMSO. Fresh or cryopreserved tumor material can be deactivated or lysed to facilitate engulfment by the dendritic cell and ensure that the final infusion product does not contain living cancer cells. The deactivation can be performed by irradiation, chemical treatment, high temperature, or a combination thereof. Tumor-infiltrating T cells can be obtained from the single-cell suspension using CD3 positive selection kit, CD4+CD8 positive selection kit or negative selection kit which are available from commercial sources such as Miltenyi Biotec.

Obtaining MDDC and T cells from peripheral blood: From blood draw or leukapheresis product, T cells and monocytes can be enriched or isolated by magnetic bead-based negative or positive selection. For example, using the CliniMACS CD14 Reagent on a CliniMACS Prodigy instrument (Miltenyi Biotec), monocytes can be routinely enriched to a purity of >95%, a recovery of >80%, with cells viability rate of >95%. The enriched monocyte can be cultured in MACS GMP Cell Culture Bags (Miltenyi Biotec) using a GMP standard procedures for 7 days in a serum-free media such as AIM-V supplemented with GM-CSF (˜1000 U/ml) and IL-4 (˜500 U/ml). Cytokines can be replenished on day 4. DC generated using this procedure (called monocyte-derived DC, MoDC or MDDC) can then be exposed to autologous tumor lysate. The tumor lysate can be generated by a variety of method such as 3 to 10 rounds of freezing/thawing and 10 Gy irradiation with a 5 min-long heating step at 100° C. during the first thawing step. DC-loading with lysate can be carried out with 50-200 μg/ml of protein during 2h. The unloaded and lysate-loaded DC can then be matured with clinical-grade tumor necrosis factor-α (TNFα; ˜50 ng/ml), IFNα (1,000 IU/ml) and poly I:C (20 mg/ml) for 24 hours. The unloaded and lysate-loaded DC can then be aliquoted to 107 to 108 per aliquot and cryopreserved in autologous serum with 10% (volume/volume) dimethylsulfoxide (DMSO) using a cryo-freezing container. The cryopreserved MDDC can be thawed using stand procedure before use. The flow-through of the monocyte enrichment step, now depleted of monocytes, can be a source material to enrich a large quantity of T cells. CliniMACS CD4 GMP MicroBeads, CliniMACS CD8 GMP MicroBeads, or the mixture of the two can be used to enrich CD4+ T cells, CD8+ T cells or pan-T cells, respectively, using the CliniMACS Prodigy system. Optionally, regulatory T cells can be depleted using CliniMACS CD25 Reagent.

Enriching tumor-experienced T cells: The peripheral PD-1-high (or PD-1^{hi}) subpopulation of T cells are of interest because they may be enriched with tumor-experienced T cells. The peripheral PD-1-high T cells can be isolated using FACS or MACS. MACS has the advantage of being easily adaptable to closed, GMP-compliant system to minimize the risk of contamination of such T cells if the T cells or their expansion product will be used in human. MACS selection of PD-1-high cells (rather than all PD-1-positive cells) can be done using biotinylated anti-PD-1 antibody and CliniMACS Anti-Biotin GMP MicroBeads using the following optimization strategy.

First, the T cells and optimal concentration of biotinylated anti-PD-1 antibody can be mixed and incubated for optimal period of time (see below), after which the T cells can be washed with CliniMACS PBS/EDTA and pelleted by centrifugation. The cell pellet can be resuspended with CliniMACS Anti-Biotin Reagent (e.g., 37.5 μl anti-biotin MicroBeads in 1 ml CliniMACS Buffer per 5×10⁶ T cells), incubated for 30 min in the dark at 2-8° C. and washed with CliniMACS PBS/EDTA buffer. The T cells obtained this way can be called PD-1-bead-enriched T cells.

A series of pilot experiment can be done to determine the optimal concentration of biotinylated anti-PD-1 antibody and optimal incubation time with this antibody so that PD-1-high T cells are sufficiently enriched. First, an aliquot of T cells can be stained with (1) biotin-labeled anti-PD-1 antibody or (2) biotin-labeled isotype control. After washing, each of these two samples can be further stained with fluorescent-labeled streptavidin (e.g., phycoerythrin-streptavidin), washed, and analyzed with flow cytometry. A “PD-1 fluorescence threshold” can be determined such that 99% of the T cells stained with biotin-labeled isotype control have fluorescent signal below this PD-1 fluorescence threshold. The median fluorescence intensity (MFI) of the of the T cells stained with biotin-labeled anti-PD-1 antibody that exceed the PD-1 fluorescence threshold can be noted as “MFIPD-1-Pos”. The PD-1-bead-enriched T cells can be stained with fluorescent-labeled streptavidin, washed and analyzed with flow cytometry. The median fluorescence intensity of the PD-1-bead-enriched T cells can be noted as “MFIPD-1-bead-enriched”. The concentration of the biotinylated anti-PD-1 antibody and incubation time can be optimized so that MFIPD-1-bead-enriched is greater than MFIPD-1-Pos by at least 5-fold. The concentration of the biotinylated anti-PD-1 antibody can be varied first logarithmically (e.g., 0.001, 0.01, 0.1, 1, 10, or 100 μg/mL) to identify a range then linearly within this range. The incubation time can be set at 30 min, but if necessary, can be optimized between 5 minutes and 2 hours with 5- to 10-min interval.

An optional method to ensure the high expression level of PD-1 among the PD-1-enriched T cells can be replacing biotin-labeled anti-PD-1 antibody with biotin-/fluorescence-doubled-labeled anti-PD-1 antibody. After the bead-based PD-1-enrichment described above, the T cells can be further FACS sorted based on the fluorescence labeled on the anti-PD-1 antibody, where only T cells exhibiting PD-1-associated fluorescent signal higher than a pre-determined threshold are sorted. The pre-determined threshold can be 4 times MFIPD-1-Pos, which is determined using the method described above, except that the isotype control is also biotin-/fluorescence-doubled-labeled and the staining with fluorescent-labeled streptavidin can be omitted.

To manufacture the biotin-labeled anti-PD-1 antibody or biotin-/fluorescence-doubled-labeled anti-PD-1 antibody, an anti-PD-1 antibody approved for therapeutic use or human in vivo clinical trial can be used, such as nivolumab, pembrolizumab, cemiplimab, sintilimab, tislelizumab, CS1003, and camrelizumab. Biotin and the fluorescence label can be conjugated to the anti-PD-1 antibody using standard coupling chemistry such as via NHS ester. For example, NHS-(PEG)12-biotin or a 1:1 (molar ratio) mixture of NHS-(PEG)12-biotin and NHS-(PEG)12-fluorescein can be mixed with the anti-PD-1 antibody in an amine-free buffer for 10 to 30 min. The coupling reaction can be quenched by amine-containing buffer.

### Example 6: Expressing Multiple Exogenous MHC Alleles in a Cell Line

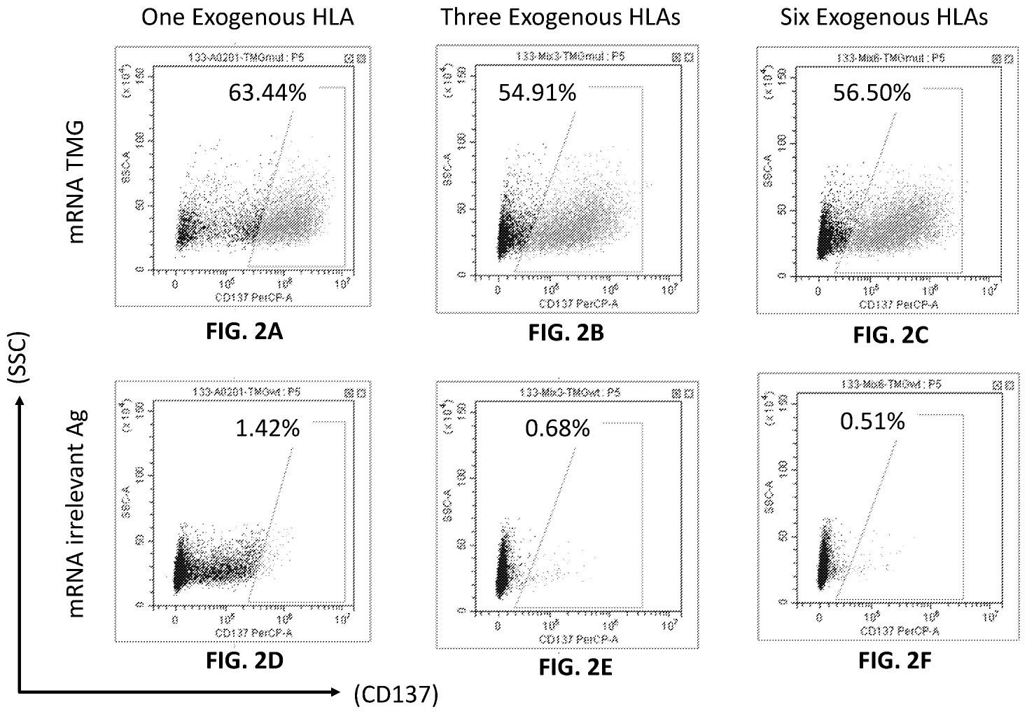

MHC genes can be highly expressed in cells. High expression level of MHC proteins may help present antigens expressed at low level. Exogenously expressed MHC gene may not reach sufficiently high expression level, if multiple exogenous MHC alleles are expressed in a cancer cell line. To test this, 1 μg of mRNA encoding HLA-A*02:01 were electroporated into K562 cells along with either (a) an mRNA of a tandem minigene (TMG) encoding several epitopes including an HLA-A*02:01-restricted NY-ESO-1 epitope (FIG. 2A), or (b) an mRNA encoding an irrelevant epitope (FIG. 2D), and co-cultured these engineered K562 cells with T cells engineered with a TCR that can recognize the A*02:01-restricted NY-ESO-1 epitope (referred to as anti-NY-ESO-1 TCR-T cells). The data shows that the former (FIG. 2A), but not the latter (FIG. 2D) engineered K562 cells can strongly stimulate the anti-NY-ESO-1 TCR-T cells. In addition, two other mRNAs encoding two other Class I MHC alleles (HLA-A*24:02, HLA-B*38:02) were added to the mRNA encoding HLA-A*02:01. The total amount of mRNAs encoding MHC alleles was kept at 1 μg, so the amount of each of the 3 MHC-encoding mRNAs was 0.33 Surprisingly, the level of stimulation of anti-NY-ESO-1 TCR-T cells was essentially unaffected (FIG. 2B, compared to FIG. 2A), although the background stimulation by K562 expressing the irrelevant antigen was somewhat reduced (FIG. 2E). Next, three more mRNAs encoding three more Class I MHC alleles (HLA-B*46:01, HLA-C*01:02, HLA-C*07:02) were added, making the total number of exogenous Class I MHC alleles six. The total amount of mRNAs encoding MHC alleles were kept at 1 μg, therefore the amount of mRNA encoding each MHC allele was only 0.167 Surprisingly, the level of stimulation of anti-NY-ESO-1 TCR-T cells remained largely unaffected (FIG. 2C). The background stimulation was also similar to the previous group (FIG. 2F). Since in this example each person has six Class I MHC alleles, the results shown here suggests that mRNAs encoding all six alleles can be introduced to a cell line and still reach sufficient expression level and sufficient antigen presentation capability.

FIGS. 2A-2F depict experimental data showing that multiple exogenous MHC alleles can be co-expressed in a cell line and achieve sufficient expression level and sufficient ability to present intracellularly expressed antigens. Anti-NY-ESO-1 TCR-T cells were co-cultured with K562 cells that were co-electroporated with (1) either (a) an mRNA of a tandem minigene (TMG) encoding several epitopes including an HLA-A*02:01-restricted NY-ESO-1 epitope, or (b) an mRNA encoding an irrelevant epitope, and (2) (i) an mRNA encoding HLA-A*02:01, (ii) an mRNA encoding HLA-A*02:01 and two other mRNAs encoding two other Class I MHC alleles, or (iii) an mRNA encoding HLA-A*02:01 and five other mRNAs encoding five other Class I MHC alleles. The total amount of mRNA encoding HLA allele(s) was kept constant at 1 μg. After 1 day of co-culture, the anti-NY-ESO-1 TCR-T cells were stained with anti-CD137 antibody and examined with flow cytometry. Only CD8⁺ anti-NY-ESO-1 TCR-T cells are shown. The percentage of anti-NY-ESO-1 TCR-T cells that are CD137⁺ are reported in the figure. SSC indicates side scattering.

### Example 7: Expressing Functional Exogenous MHC Alleles in an MHC-Null Cell Line

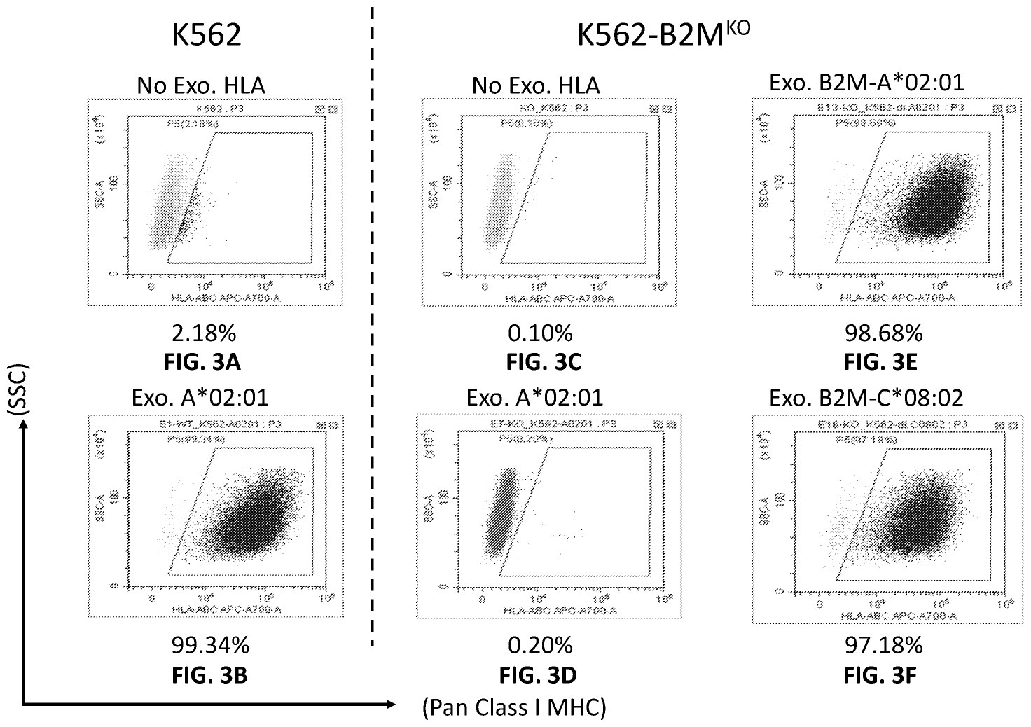

Non-classical MHC alleles such as HLA-E and HLA-G can be introduced to MHC-null cells to avoid recognition or killing by NK cells. However, whether classical MHC alleles can be introduced to MHC-null cells (especially those obtained by B2M knock-out) and function properly in presenting antigens may be less clear. To test this, K562 cell line was used as a model cell line and studied whether exogenous MHC alleles can be expressed and function. K562 cells generally can have low level of MHC-I-alpha expression. This was confirmed in FIG. 3A. However, when an mRNA encoding an exogenous MHC allele, HLA-A*02:01, was transfected into K562 by electroporation, an abundant amount of Class I MHC was detected on the cell surface (FIG. 3B). The data confirm the quality and function of mRNA encoding HLA-A*02:01. Next, B2M was knocked out in K562 using CRISPR/Cas9 to produce K562-B2M K°. The data in FIG. 3C, compared to the data in FIG. 3A, show the success of the knock-out of B2M. When the mRNA encoding HLA-A*02:01 was electroporated into K562-B2M K°, cell surface Class I MHC remained undetectable (FIG. 3D). However, when an mRNA encoding B2M-HLA-A*02:01 fusion (FIG. 3E) or B2M-HLA-C*08:02 fusion (FIG. 3F) (as examples of B2M-MHC-I-alpha fusion) was electroporated into K562-B2M K°, abundant cell surface Class I MHC was once again detected, indicating that exogenous B2M-MHC-I-alpha fusion can be expressed and transported to cell surface in MHC-null cells.

FIGS. 3A-3F depict experimental data showing that B2M-MHC-I-alpha fusion can be abundantly expressed and transported to cell surface in MHC-null cells. Either parental K562 cells or K562-B2M^{KO} cells were stained with an antibody recognizing human pan Class I MHC (FIG. 3A and FIG. 3C). These two cell lines were also transfected with mRNA encoding HLA-A*02:01, B2M-HLA-A*02:01 fusion, or B2M-HLA-C*08:02 fusion and stained the same way (FIG. 3B, FIG. 3D, FIG. 3E and FIG. 3F). The percentage of positively stained cells are reported in the figure. SSC indicates side scattering.

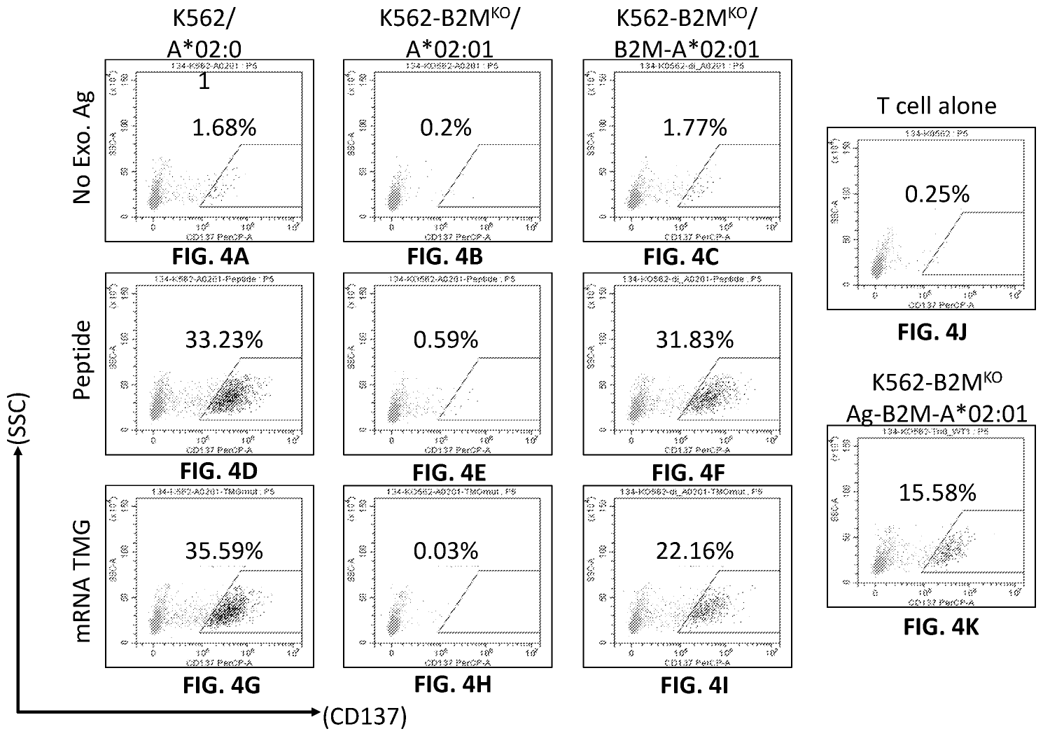

T cells engineered with a TCR that recognizes HLA-A*02:01-restricted WT-1 epitope (referred to as anti-WT-1 TCR-T cells) was used as a sensor to test whether the B2M-MHC-I-alpha fusion can present intracellularly expressed antigens. The cell surface CD137 on the anti-WT-1 TCR-T cells can be upregulated after the TCR-T cell is stimulated by target cell through TCR signaling. Parental K562 cells were electroporated with mRNA encoding HLA-A*02:01 to form K562/A*02:01. K562-B2M^{KO} cells were electroporated with mRNA encoding HLA-A*02:01 or B2M-HLA-A*02:01 to form K562-B2M^{KO}/A*02:01 or K562-B2M^{KO}/B2M-A*02:01, respectively. As negative controls, none of these three MHC-engineered cell lines stimulated anti-WT-1 TCR-T cells (FIG. 4A, FIG. 4B and FIG. 4C), compared to anti-WT-1 TCR-T cells without co-culture (FIG. 4J). When WT-1 peptide was added to the culture media, K562/A*02:01 (FIG. 4D) and K562-B2M^{KO}/B2M-A*02:01 (FIG. 4F), but not K562-B2M^{KO}/A*02:01 (FIG. 4E), can stimulate the T cells, indicating the importance of B2M-MHC-I-alpha fusion in the B2M^{KO} background. Similarly, when an mRNA of a tandem minigene (TMG) encoding several epitopes including the WT-1 epitope was co-transfected with the mRNA encoding HLA*02:01 or B2M-HLA*02:01, it can be seen that K562/A*02:01 (FIG. 4G) and K562-B2M^{KO}/B2M-A*02:01 (FIG. 4I), but not K562-B2M^{KO}/A*02:01 (FIG. 4H), can stimulate the T cells, again demonstrating the importance of B2M-MHC-I-alpha fusion and its ability to present intracellularly expressed antigens. Surprisingly, this level of activation is similar to that achieved by a fusion protein comprising antigen peptide, B2M, and MHC-I-alpha (referred to as Ag-B2M-A*02:01, see FIG. 4K) expressed from an electroporated mRNA, which has been used to present antigens as an artificially high level (since all exogenously expressed HLA is physically linked to its antigen). Overall, the B2M-WIC-I-alpha fusion demonstrated surprisingly strong capability to present intracellularly expressed antigens on a B2M^{KO} background.

FIGS. 4A-4K depict experimental data showing that B2M-WIC-I-alpha fusion can efficiently present intracellularly expressed antigens in WIC-null cells. Various version of K562 cells were co-cultured with anti-WT-1 TCR-T cells. After co-culture for 1 day, the anti-WT-1 TCR-T cells were stained with anti-CD137 antibody and examined by flow cytometry. In FIG. 4A, FIG. 4D and FIG. 4G, parental K562 cells were electroporated with an mRNA encoding HLA-A*02:01. In FIG. 4B, FIG. 4E and FIG. 4H, K562-B2M^{KO} cells were electroporated with an mRNA encoding HLA-A*02:01. In FIG. 4C, FIG. 4F and FIG. 4I, K562-B2M^{KO} cells were electroporated with an mRNA encoding B2M-HLA-A*02:01 fusion. In FIG. 4K, K562-B2M^{KO} cells were electroporated with an mRNA encoding antigen-B2M-HLA-A*02:01 fusion, where the antigen is the WT-1 epitope recognized by the anti-WT-1 TCR-T cells. In FIG. 4D, FIG. 4E and FIG. 4F, the WT-1 epitope peptide was added in the co-culture media. In FIG. 4G, FIG. 4H and FIG. 4I, an mRNA of a TMG encoding several epitopes including the WT-1 epitope was co-electroporated to the K562 cells (or the derivative thereof) along with the mRNA encoding the WIC allele (or the derivative thereof). FIG. 4J shows the CD137 level of the anti-WT-1 TCR-T cells without co-culture. SSC indicates side scattering.

### Example 8: Presenting Endogenous Antigens by B2M-MHC-I-Alpha Fusion

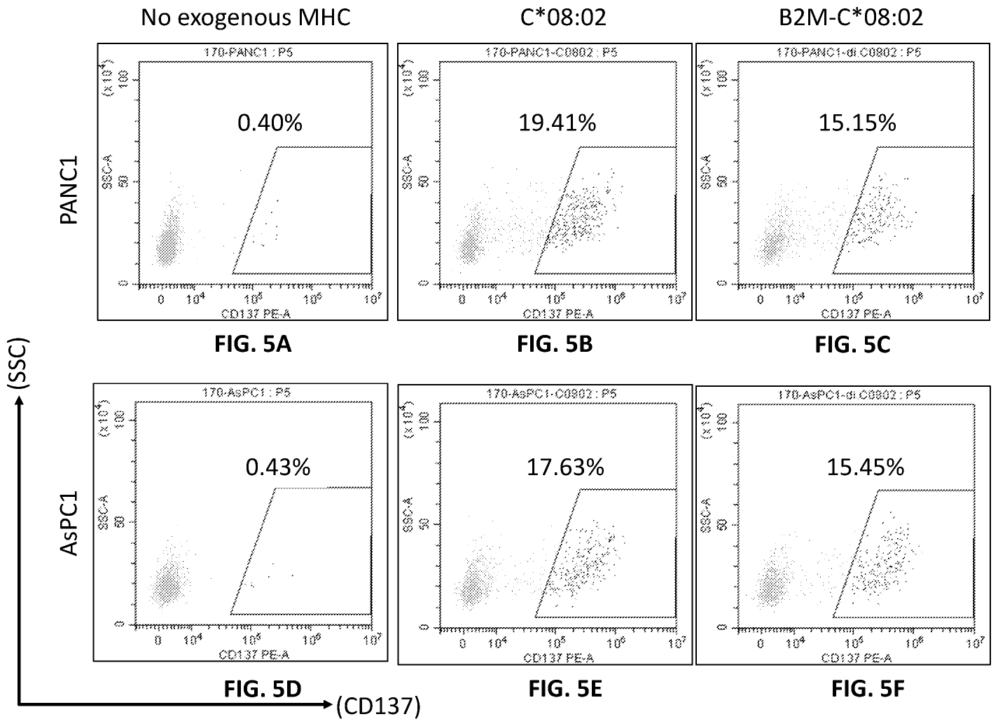

The previous example shows that B2M-WIC-I-alpha fusion can present intracellularly expressed antigen for T cell recognition. To further demonstrate that B2M-WIC-I-alpha fusion can present endogenous antigens (e.g., antigens expressed from the cell line's natural or endogenous genome) for T cell recognition, PANC1 and AsPC1 cell lines and a known TCR (NCI4095-2) which recognizes a C*08:02-restricted KRAD G12D epitope were used. Both PANC1 and AsPC1 carry the KRAS^{G12D} mutation, but neither expresses HLA-C*08:02. The NCI4095-2 TCR was transduced to the peripheral T cells of a donor to form anti-KRAS^{G12D} TCR-T cells. As shown in FIGS. 5A-5F, although the anti-KRAS^{G12D} TCR-T cells do not recognize PANC1 or AsPC1, both cell lines can be recognized by the anti-KRAS^{G12D} TCR-T cells when exogenous HLA-C*08:02 or exogenous B2M-C*08:02 fusions were expressed in these cell lines via an mRNA vector.

FIGS. 5A-5F depict experimental data showing that B2M-WIC-I-alpha fusion can efficiently present endogenous antigens in cancer cells. Various version of PANC1 and AsPC1 cells was co-cultured with anti-KRAS^{G12D} TCR-T cells. Both PANC1 and AsPC1 carry the KRAS^{G12D} mutation, but neither expresses HLA-C*08:02. The anti-KRAS^{G12D} TCR-T cells recognize C*08:02-restricted KRAS^{G12D} peptide. After co-culture for 1 day, the anti_KRAS^{G12D} TCR-T cells were stained with anti-CD137 antibody and examined by flow cytometry. In FIG. 5A, PANC1 cells were not engineered with exogenous MEW. In FIG. 5B, PANC1 cells were engineered to express exogenous HLA-C*08:02 alpha chain. In FIG. 5C, PANC1 cells were engineered to express exogenous B2M-C*08:02 fusion. In FIG. 5D, AsPC1 cells were not engineered with exogenous MEW. In FIG. 5E, AsPC1 cells were engineered to express exogenous HLA-C*08:02 alpha chain. In FIG. 5F, AsPC1 cells were engineered to express exogenous B2M-C*08:02 fusion. SSC indicates side scattering.

### Example 9: Expression Kinetics of MHC-I in Cancer Cell Lines

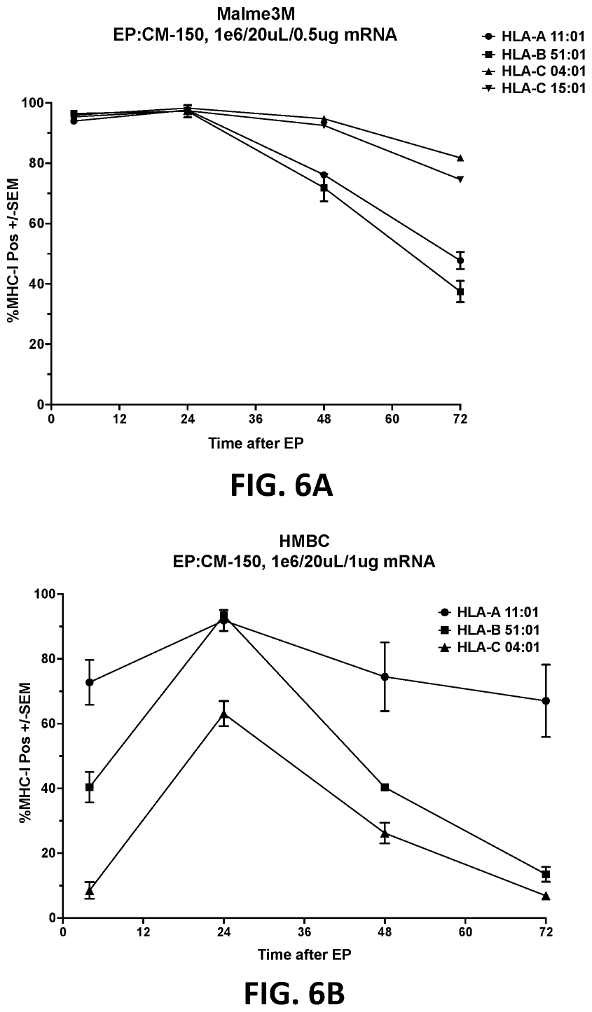

FIG. 6A and FIG. 6B show expression kinetics of MHC-I in cancer cell lines. Melanoma cell lines Malme3M (FIG. 6A) and HMCB (FIG. 6B) were edited at the B2M locus to produce MHC-null cells. Various B2M-MHC-I-alpha fusion alleles were introduced by mRNA transient transfection and surface expression monitored over time by pan MHC-I antibody. The total amount of mRNA was kept constant for each MHC-I, and the cells were transfected separately. The surface expression of HLA-A 11:01, HLA-B 51:01, HLA-C 04:01 and HLA-C 15:01 were assayed in Malme3M and the surface expression of HLA-A 11:01, HLA-B 51:01 and HLA-C 04:01 were assayed in HMCB at the timepoints indicated in FIG. 6A and FIG. 6B, respectively.

### Example 10: An Example Workflow of TCR Identification Using Synthetic Library and Cancer Cell Line

T cells, via their T cell receptor (TCR), can bind antigen presented in the context of MEW in a highly specific manner. A synthetic TCR library (e.g., a library containing about 1,000 natively paired TCRs) can be expressed in normal donor T cells to generate a synthetic library of TCR-T cells, and cells that are specifically recognizing the APC or tumor cell can be enriched by sorting the TCR-T cells.

Normal donor T cells can be isolated, activated by CD3/CD28, and then engineered by lentivirus or adeno-associated virus of a synthetic TCR library. Once these T cells have fully expanded and have stopped proliferating, they are either frozen for later use or directly used in co-culture assay. The co-culture comprises an APC such as a monocyte derived dendric cells, B cell, primary tumor material, or cancer cell line mixed with the TCR-T cell library and incubated for 4-24 hrs. After the incubation time, the co-culture cells are then stained for an activation marker such as CD137, OX40, CD107a, etc. Next, a portion of the co-culture cells are set aside for the “pre-sorted” sample; these will be wash and frozen to be processed for sequencing later. The rest of the co-culture is then sorted by either a bead-based enrichment protocol or fluorescent activation cell sorting (FACS) using an activation marker. The “sorted” cell samples will be wash, frozen, and then processed later.

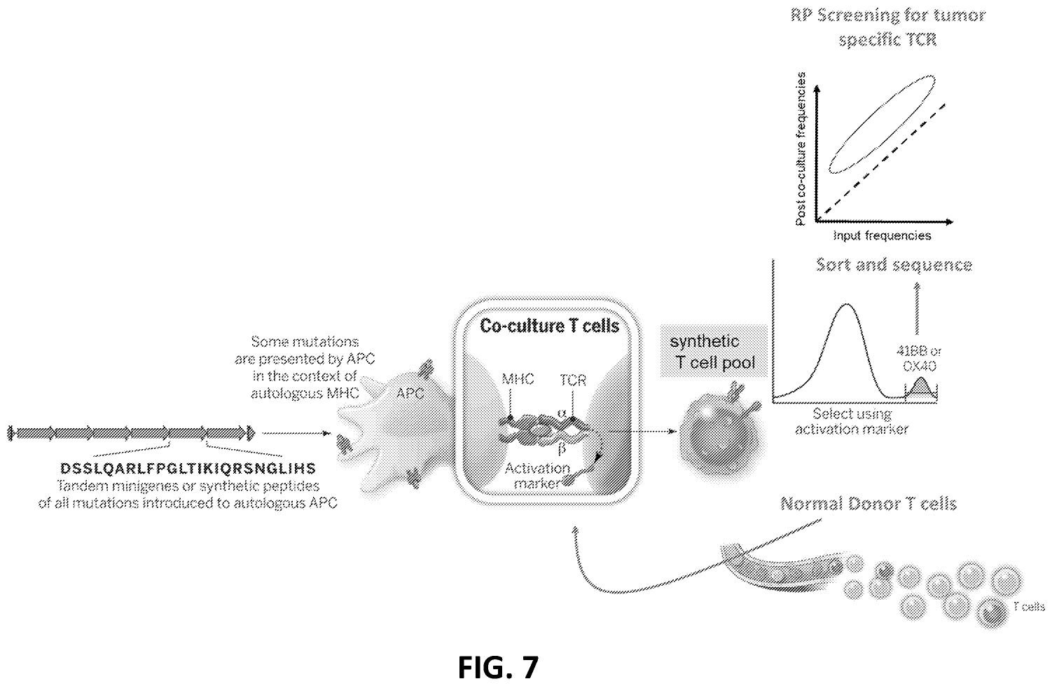

Next, the pre-sorted T cells and the sorted T cells are sequenced by next generation sequencing (NGS). Genomic DNA or RNA are isolated and used in PCRs to generate libraries for NGS on an Illumina sequencer. Custom primers produce NGS reads of CDR3 region specifically. The raw reads counts are obtained by aligning to the synthetic TCR library. Target-reactive TCRs (e.g., tumor antigen reactive TCRs) are defined by comparing pre-sorted to post-sorted frequencies and/or fold change as shown in the volcano plots (FIG. 7).

### Example 11: TCR Identification Using Synthetic Library and Cancer Cell Line

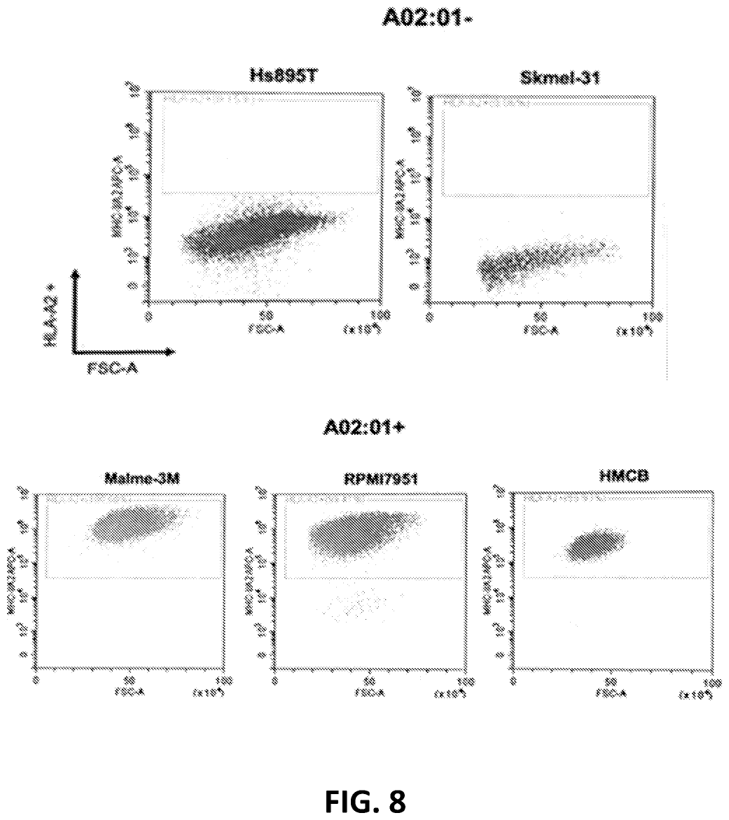

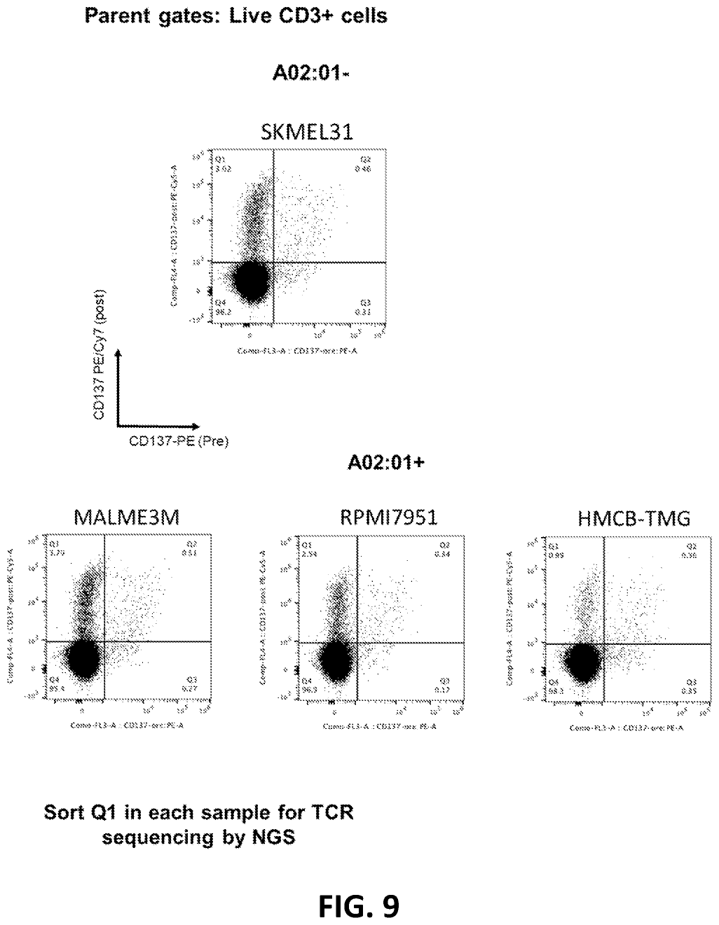

Using the workflow outlined in Example 10, a synthetic TCR-T cell library can be screened against antigens for a specific HLA restriction. As an example, cancer cell lines that either expressing or are negative for HLA-A02:01 were used to identify TCRs that are only reactive to antigens restricted by HLA-A02:01 (FIG. 8). CD137 was used as the marker for reactivity or activation. Residual CD137 expression was observed on the TCR-T cells prior to activation. To increase selection sensitivity of CD137, the TCR-T cells were stained with CD137-PE before setting up the co-culture with the cancer cell lines and then after co-culture the cells were stained with the same clone of CD137 but with PE/Cy7. This method was used to sort TCR-T cells that were only newly activated by the cancer cell line and reduced the background from any residual CD137 expression. FIG. 9 shows the flow cytometry plots from four different co-cultures where the cells displayed are live synthetic TCR-T cells stained with “pre” and “post” CD137. The cells sorted and sequenced are the population in Q1—the TCR-T cells newly activated by the cancer cell line indicated. As a positive control, one of the cancer cell lines that are HLA-A02:01 positive was electroporated with tandem mini genes (TMGs) that contain the antigen of the model TCRs (e.g., PMEL17, DMF5, 1G4, NKI.CDK4.53, and DMF4) contained in the library.

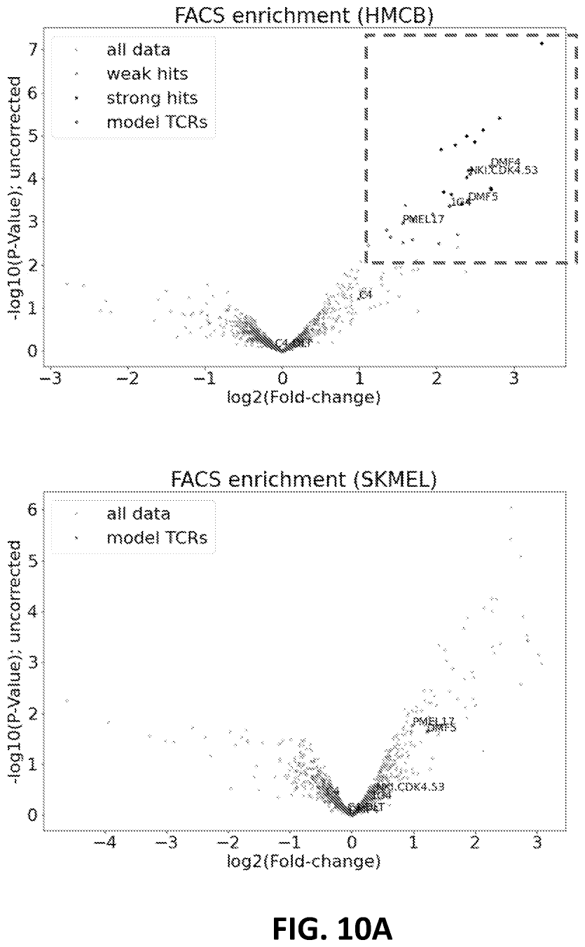

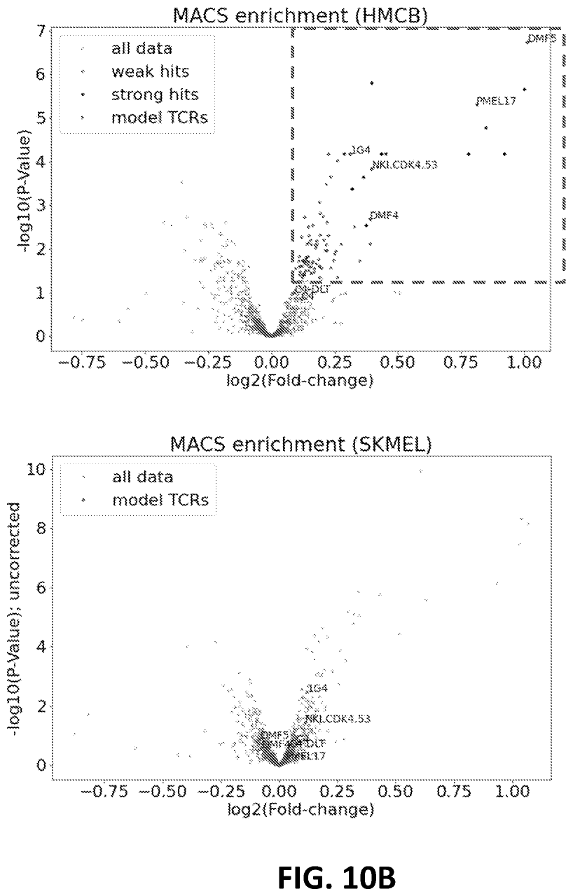

Next, the sorted TCR-T cells were sequenced and then “hits” were defined as combination of fold-change and significance threshold of fold change in frequencies from the input and significance cutoff. The volcano plots of FACS data (FIG. 10A) show that in the model TCRs along with other unknown TCRs were enriched (see data points within the box with dotted line) in the positive control co-culture with HMCB-TMG but were not enriched in the HLA-A02:01 negative cell line SKMEL. Additionally, a bead-based CD137 enrichment using MACS was also used (FIG. 10B). Similar results as in FACS were observed. These results suggest that unknown HLA-A02:01 specific TCRs in the synthetic library can be detected. 96 TCRs were chosen for further validation. Among the 96 TCRs, 64 TCRs were only enriched in the HLA-A02:01 positive cancer cell lines but not in the HLA-A02:01 negative cancer cell line SKMEL. 16 TCRs were enriched in all the cancer cell lines tested and then another 20 TCRs that showed no enrichment where chosen as negative controls.

### Example 12: Validation of Identified TCRs in Example 11

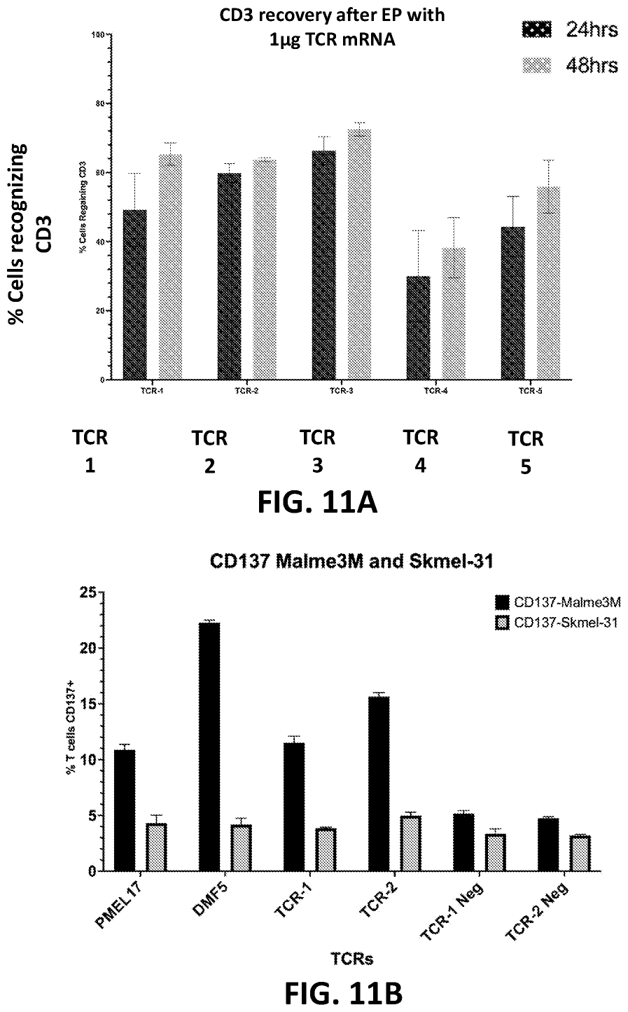

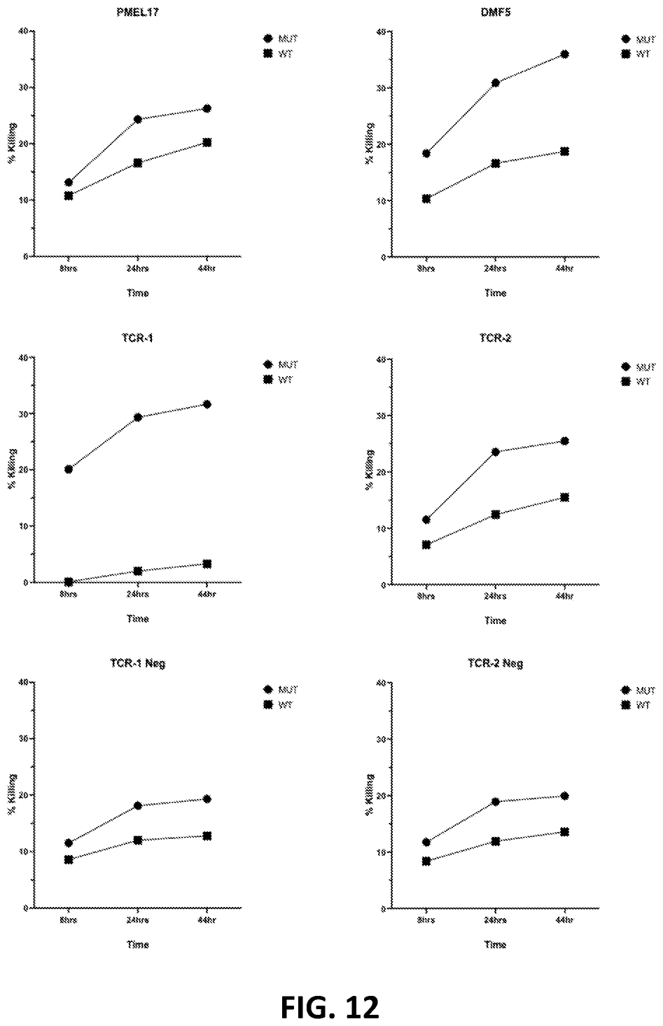

Specific TCRs of interests can be amplified using unique primers from the original pool of 1,000 different TCRs. For the validation, 96 primers that are specific for the 96 TCRs identified above were used. PCR and in vitro transcription (IVT) were performed to generate a 96-well plate of mRNA of individual TCRs. Next, the normal donor T cells that have been previously engineered to not express a TCR by knocking-out TRAC and TRBC with CRIPSR/Cas9 (refer to as double knock-out (DKO)) were used to express the identified TCRs. These DKO cells were electroporated with mRNA of each TCR using the Lonza 96-shuttle system. The highest recovery of CD3 was observed 48 hrs post electroporation (EP), indicating TCR expression (FIG. 11A). The DKO cells expressing the identified TCRs were co-cultured with HLA-A02:01 positive or negative cancer cell line and the percentage of the activated population of cells were determined by CD137 upregulation (FIG. 11B). The TCRs were further validated using a killing assay, where T cells expressing the identified TCRs were co-cultured with APCs. The APCs are HLA-A02:01 positive expressing a tandem mini gene (TMG) containing known antigens (MUT) or other antigens (WT) (FIG. 12).

### Example 13: TCR Identification Using Synthetic Library and Cancer Cell Line Expressing Patient-Specific HLAs from Carcinoma Patient

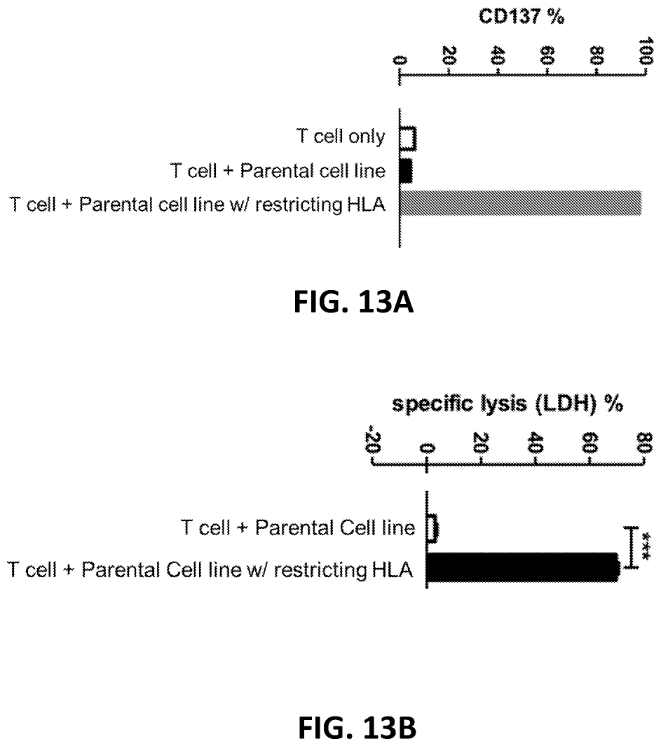

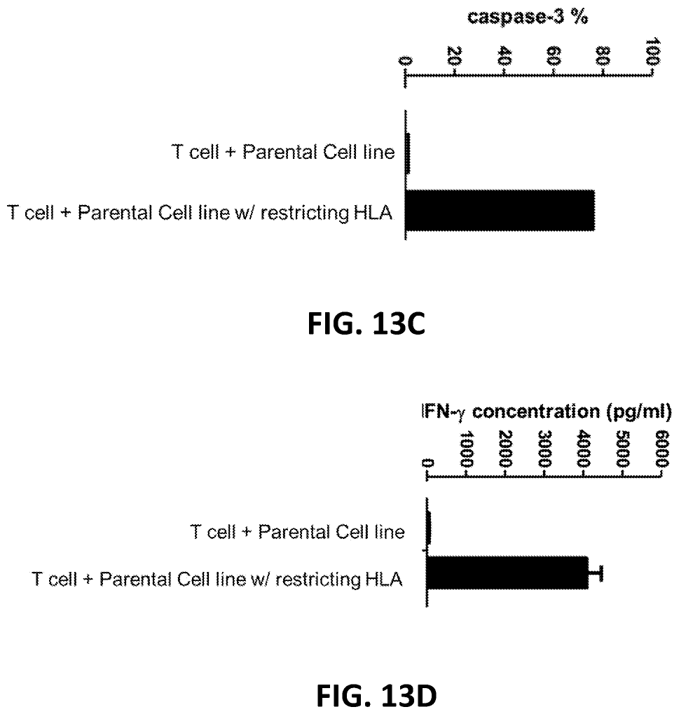

A solid tumor from a hepatocellular carcinoma patient was surgically removed and processed into single cells. T cells were positively selected by the surface marker CD3 and the selected fraction was subjected to single-cell RNA sequencing. The paired TCR information was then used to synthesize all the TCRs observed in this dataset and the paired TCR clones were engineered into donor T cells to generate engineered T cells. A cancer cell line from the same indication as that of the patient was edited to create MHC-null cells as shown above and transfected with all six class I HLA alleles (e.g., subject-specific HLAs) from the patient. The engineered T cells containing the paired TCRs from the patient and the cancer cell line presenting the six class I HLA alleles from the same patient were then cultured together and antigen-reactive T cells were sorted for activation based on new CD137 expression. The sorted cells were sequenced and analyzed for fold enrichment when compared to initial frequencies. Those showing high fold enrichment are designated as hits and subjected to further validation individually. Several of these TCRs were validated as reactive to the patient HLA engineered cell line. A representative TCR was selected for further investigation. First, individual HLA was transfected into the cell line to determine the HLA restriction. Two closely related HLA were found to activate T cells containing the TCR. One of the two HLA, which leads to stronger activation of the T cell containing the TCR, was designated as the restricting HLA. FIG. 13A shows the upregulation of an early activation marker CD137 only in response to the parental cell line expressing the patient's restricting HLA. FIG. 13B shows the results of a cell lysis experiment as monitored by an lactate dehydrogenase (LDH) assay, where increased signal is directly related to lysis and increased levels of the LDH enzyme. The investigated TCR expressing T cells only lysed the target cell line when expressing the patient's restricting HLA. FIG. 13C shows another co-culture assay where apoptosis was monitored by a CaspaseGlo® 3/7 assay. Apoptosis of the parental cell line was only observed when the patient's restricting HLA is expressed. FIG. 13D shows another co-culture assay where T cell activation was monitored by cytokine release. In this experiment, concentration of released IFN-γ was determined. Release of high amount of IFN-γ from the T cells was observed when the patient's restricting HLA is expressed.

### Example 14: TCR Identification Using Synthetic Library and Cancer Cell Line Expressing Patient-Specific HLAs from Melanoma Patient

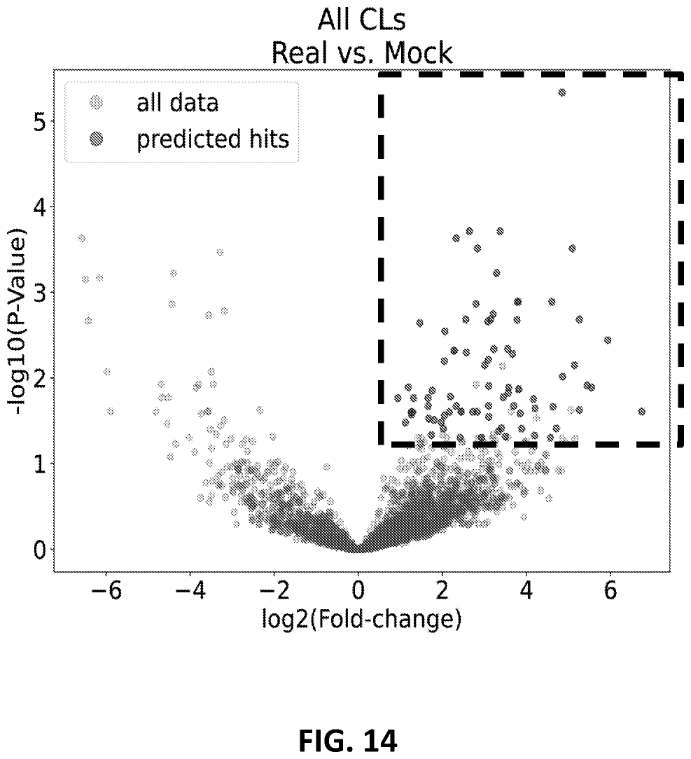

The blood of a late-stage melanoma patient was collected after checkpoint therapy. T cells expressing PD1 were sorted and subjected to single-cell RNA sequencing. The paired TCR information of sorted cells was then used to synthesize TCRs observed in the dataset and the paired TCR clones were engineered into a donor T cells. Two cancer cell lines from the same patient indication were edited to create MHC-null cells as shown above and transfected with all six class I HLA alleles from the patient (positive selection) or six unrelated HLA alleles (negative selection). The engineered T cells containing the paired TCRs from the patient and the cancer cell lines presenting the class I HLA alleles were then cultured together and T cells reactive to either the negative or positive selection were sorted based on new CD137 expression. The sorted cells were sequenced and analyzed for reactivity to either cell line. The volcano plot (FIG. 14) shows the maximum value for either cell line for individual TCR sequences as a function of fold enrichment (compared to pre-selection frequencies) and P value. TCR sequences showing high statistical enrichment that are not present in the negative selection are designated as hits (see data points within the box with dotted line) and subjected to further validation individually. This analysis shows the ability to use patient HLA engineered cell lines to discover TCR sequences from the peripheral blood of a patient that are potentially reactive to the patient's cancer.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### Embodiment Paragraphs

The present disclosure provides:

A method for identifying an antigen-reactive cell that recognizes an endogenous antigen of a cancer cell line in complex with an MHC molecule expressed by a subject, comprising:
(a) providing a cell that is a cancer cell line expressing an endogenous antigen in complex with an exogenous MHC molecule, wherein the exogenous MHC molecule is the MHC molecule expressed by the subject or derived from the subject;
(b) contacting the cancer cell line with a first plurality of TCR-expressing cells, wherein the first plurality of TCR-expressing cells or a subset of the first plurality of TCR-expressing cells is activated by the endogenous antigen in complex with the exogenous MHC of the cancer cell line; and
(c) subsequent to contacting in (b), identifying the subset of the first plurality of TCR-expressing cells, thereby identifying the antigen-reactive cell that recognizes the endogenous antigen of the cancer cell line.

The method of paragraph [001], wherein identifying in (c) comprises enriching or selecting the subset of the first plurality of TCR-expressing cells.

The method of paragraph or [002], wherein the exogenous MHC molecule is exogenous to the cancer cell line.

The method of any one of paragraphs [001]-[003], wherein the method further comprises, in (a), providing a non-cancer cell expressing an additional endogenous antigen in complex with an exogenous MHC molecule, wherein the exogenous MHC molecule is derived from the same subject.

The method of paragraph [004], further comprising, in (b), contacting the non-cancer cell with a second plurality of TCR-expressing cells, and wherein a subset of the second plurality of TCR-expressing cells is activated by the additional endogenous antigen in complex with the exogenous MHC of the non-cancer cell.

The method of paragraph or [005], wherein the additional endogenous antigen is the same as or different from the endogenous antigen expressed by the cancer cell line.

The method of any one of paragraphs [004]-[006], wherein the non-cancer cell (i) does not express the endogenous antigen expressed by the cancer cell line, (ii) expresses the endogenous antigen expressed by the cancer cell line at a lower level, or (iii) expresses the endogenous antigen expressed by the cancer cell line, but does not present the endogenous antigen expressed by the cancer cell line.

The method of any one of paragraphs [005]-[007], wherein the first plurality and the second plurality of TCR-expressing cells are derived from a same sample.

The method of any one of paragraphs [005]-[008], wherein the first plurality and the second plurality of TCR-expressing cells express a same TCR.

The method of any one of paragraphs [005]-[009], wherein the first plurality or the second plurality of TCR-expressing cells expresses different TCRs.

The method of any one of paragraphs [005]-[010], further comprising, in (c), identifying the subset of the second plurality of TCR-expressing cells.

The method of any one of paragraphs [001]-[011], wherein identifying comprises selecting the subset of the first plurality of TCR-expressing cells and/or the subset of the second plurality of TCR-expressing cells based on a marker.

The method of paragraph [012], wherein selecting the subset of the first plurality of TCR-expressing cells and/or the subset of the second plurality of TCR-expressing cells comprises using fluorescence activated cell sorting (FACS) or magnetic activated cell sorting (MACS) based on the marker.

The method of paragraph [013], further comprising identifying a TCR that is expressed in the subset of the first plurality of TCR-expressing cells.

The method of paragraph [013], further comprising identifying a TCR that is expressed in the subset of the first plurality of TCR-expressing cells, but not in the subset of the second plurality of TCR-expressing cells.

The method of paragraph [013], further comprising identifying a TCR of a cell in the subset of the first plurality of TCR-expressing cells that is activated by the endogenous antigen in complex with the exogenous MHC of the cancer cell line, and that is in a cell in the second plurality of TCR-expressing cells that is not activated by the additional endogenous antigen in complex with the exogenous MHC of the non-cancer cell.

The method of any one of paragraphs [004]-[016], wherein the non-cancer cell is a stem cell or a primary cell.

The method of paragraph [017], wherein the stem cell is an induced pluripotent stem cell (iPSC).

The method of paragraph [018], wherein the non-cancer cell is an differentiated iPSC.

The method of any one of paragraphs [004]-[019], wherein the non-cancer cell expresses an autoimmune regulator (AIRE).

The method of any one of paragraphs [001]-[019], wherein an endogenous MHC molecule of the cancer cell line or the non-cancer cell is inactivated (e.g., knocked down, or knocked out).

The method of any one of paragraphs [001]-[021], wherein the cancer cell line or non-cancer cell is null for an endogenous MEW molecule.

The method of any one of paragraphs [001]-[022], wherein the cancer cell line or non-cancer cell is null for all endogenous MHC molecules.

The method of any one of paragraphs [021]-[023], wherein the endogenous MEW molecule comprises a MHC class I molecule, a MEW class II molecule, or a combination thereof.

The method of paragraph [024], wherein the MEW class I molecule comprises HLA-A, HLA-B, HLA-C, or any combination thereof.

The method of paragraph or [025], wherein an alpha chain of the MHC class I molecule (MHC-I alpha) is inactivated.

The method of paragraph [026], wherein a gene encoding the alpha chain of the MEW class I molecule is inactivated.

The method of any one of paragraphs [024]-[027], wherein a beta-2-microglobulin (B2M) of the MHC class I molecule is inactivated.

The method of paragraph [028], wherein a gene encoding the B2M of the MEW class I molecule is inactivated.

The method of any one of paragraphs [024]-[029], wherein the MHC class II molecule comprises HLA-DP, HLA-DM, HLA-DOA, HLA-DOB, HLA-DQ, HLA-DR, or any combination thereof.

The method of any one of paragraphs [024]-[030], wherein an alpha chain or a beta chain of the MHC class II molecule is inactivated.

The method of paragraph [031], wherein a gene encoding the alpha chain or the beta chain of the MHC class II molecule is inactivated.

The method of paragraph [031], wherein a gene regulating transcription of the MHC class II molecule is inactivated.

The method of paragraph [033], wherein the gene is CIITA.

The method of any one of paragraphs [001]-[034], wherein the exogenous MHC molecule of the cancer cell line or the non-cancer cell comprises a MHC class I molecule, a MHC class II molecule, or a combination thereof, derived from the subject.

The method of paragraph [035], wherein the MHC class I molecule comprises HLA-A, HLA-B, HLA-C, or any combination thereof.

The method of paragraph or [036], wherein the MHC class II molecule comprises HLA-DP, HLA-DM, HLA-DOA, HLA-DOB, HLA-DQ, HLA-DR, or any combination thereof.

The method of any one of paragraphs [035]-[037], wherein the exogenous MHC molecule comprises an MHC-I alpha derived from the subject and an endogenous B2M.

The method of any one of paragraphs [035]-[038], wherein the exogenous MHC molecule comprises both an MHC-I alpha and a B2M derived from the subject.

The method of paragraph [039], wherein the exogenous MHC molecule is a fusion protein of the MHC-I alpha and the B2M (B2M-MHC-I-alpha fusion).

The method of paragraph [040], wherein the MHC-I alpha and the B2M is linked by a linker.

The method of paragraph [041], wherein the linker is (G4S)n, wherein G is glycine, S is serine, and n is an integer from 1 to 10.

The method of any one of paragraphs [035]-[042], wherein the exogenous MHC molecule comprises an MHC-II alpha and an MHC-II beta derived from the subject.

The method of any one of paragraphs [001]-[043], wherein the first plurality of TCR-expressing cells is isolated from the same subject.

The method of any one of paragraphs [001]-[044], wherein the first plurality of TCR-expressing cells comprises a primary T cell.

The method of paragraph [045], wherein the primary T cell is a tumor-infiltrating T cell.

The method of paragraph [045], wherein the primary T cell is a peripheral T cell.

The method of paragraph [047], wherein the peripheral T cell is a tumor-experienced T cell.

The method of paragraph [047], wherein the peripheral T cell is a PD-1+ T cell.

The method of any one of paragraphs [045]-[049], wherein the primary T cell is a CD4+ T cell, a CD8+ T cell, or a combination thereof.

The method of any one of paragraphs [045]-[049], wherein the primary T cell is a cytotoxic T cell, a memory T cell, a national killer T cell, an alpha beta T cell, a gamma delta T cell, or any combination thereof.

The method of any one of paragraphs [001]-[051], wherein the first plurality of TCR-expressing cells comprises an engineered cell.

The method of paragraph [052], wherein the engineered cell expresses an exogenous TCR.

The method of paragraph [053], wherein the exogenous TCR is derived from a primary T cell isolated from the same subject.

The method of any one of paragraphs [001]-[054], further comprising, prior to (a), isolating a primary cancer cell or a tumor sample from the subject.

The method of paragraph [055], further comprising conducting transcriptomic or genomic analysis of the primary cancer cell or the tumor sample and cancer cell lines to identify the cancer cell line having a gene expression profile, a transcriptomic profile or a genomic alteration that resembles a primary cancer cell or the tumor sample isolated from the subject.

The method of paragraph [056], wherein a correlation coefficient of the gene expression profile, the transcriptomic profile or the genomic alteration between the cancer cell line and the primary cancer cell or the tumor sample is equal to or greater than about 0.1.

The method of any one of paragraphs [001]-[057], further comprising, in (c), identifying a TCR of the subset.

The method of paragraph [058], further comprising identifying a sequence of a TCR expressed by the antigen-reactive cell.

The method of paragraph [059], wherein identifying the sequence of the TCR comprises sequencing a TCR repertoire of the subset of the first plurality of TCR-expressing cells.

The method of any one of paragraphs [001]-[060], further comprising administering the antigen-reactive cell or a cell comprising a sequence encoding the TCR of the antigen-reactive cell into the subject.

The method of any one of paragraphs [001]-[061], wherein the first plurality of TCR-expressing cells expresses a plurality of TCRs comprising at least 10 different cognate pairs derived from the same subject.

The method of paragraph [062], wherein the plurality of TCRs comprises V regions from a plurality of V genes.

The method of any one of paragraphs [001]-[063], wherein the cell that is a cancer cell line comprises at least about 50, 100, 1,000 or more cells.

The method of any one of paragraphs [001]-[064], further comprising, prior to (b), killing the cancer cell line.

The method of paragraph [065], wherein killing comprising irradiating or treating the cancer cell line with a chemical compound.

The method of paragraph [066], wherein the chemical compound is a cytotoxic compound.

The method of paragraph [067], wherein the cytotoxic compound is cis-platin, cyclophosphamide, nitrogen mustard, trimethylene thiophosphoramide, carmustine, busulfan, chlorambucil, belustine, uracil mustard, chlomaphazin, dacabazine, cytosine arabinoside, fluorouracil, methotrexate, mercaptopuirine, azathioprime, procarbazine, doxorubicin, bleomycin, dactinomycin, daunorubicin, mithramycin, mitomycin, mytomycin C, daunomycin, or any combination thereof.

A method for identifying an antigen-reactive cell that recognizes an antigen in complex with an MHC molecule expressed by a subject, comprising:
(a) providing a cancer cell line expressing an antigen in complex with an exogenous MHC molecule, wherein the exogenous MHC molecule is the MHC molecule expressed by the subject or derived from the subject;
(b) contacting the cancer cell line with a plurality of engineered cells expressing a plurality of TCRs comprising at least 10 different cognate pairs derived from the same subject, and wherein a subset of the plurality of engineered cells is activated by the antigen in complex with the exogenous MHC of the cancer cell line; and
(c) subsequent to contacting in (b), identifying the subset of the plurality of engineered cells, thereby identifying the antigen-reactive cell.

The method of paragraph [069], wherein the antigen is endogenous to the cancer cell line.

The method of paragraph or [070], wherein the cancer cell line does not express an exogenous antigen or does not present an exogenous antigen.

The method of any one of paragraphs [069]-[071], wherein the antigen is a tumor-associated antigen (TAA) or a tumor-specific antigen (TSA).

The method of any one of paragraphs [069]-[072], wherein the cancer cell line is not derived from the same subject.

The method of any one of paragraphs [069]-[073], wherein the cancer cell line has a transcriptomic profile or genomic alteration that resembles a primary cancer cell isolated from the subject.

The method of any one of paragraphs [069]-[074], wherein the plurality of TCRs are exogenous to the plurality of engineered cells.

The method of any one of paragraphs [069]-[075], wherein an endogenous MHC molecule of the cancer cell line is inactivated (e.g., knocked down, or knocked out).

The method of paragraph [076], wherein the endogenous MHC molecule comprises a MHC class I molecule, a MHC class II molecule, or a combination thereof.

The method of paragraph [077], wherein the MHC class I molecule comprises HLA-A, HLA-B, HLA-C, or any combination thereof.

The method of paragraph or [078], wherein an alpha chain of the MHC class I molecule (MHC-I alpha) is inactivated.

The method of paragraph [079], wherein a gene encoding the alpha chain of the MHC class I molecule is inactivated.

The method of any one of paragraphs [077]-[080], wherein an beta-2-microglobulin (B2M) of the MHC class I molecule is inactivated.

The method of paragraph [081], wherein a gene encoding the B2M of the MHC class I molecule is inactivated.

The method of any one of paragraphs [077]-[082], wherein the MHC class II molecule comprises HLA-DP, HLA-DM, HLA-DOA, HLA-DOB, HLA-DQ, HLA-DR, or any combination thereof.

The method of any one of paragraphs [077]-[083], wherein an alpha chain or a beta chain of the MHC class II molecule is inactivated.

The method of paragraph [084], wherein a gene encoding the alpha chain or the beta chain of the MHC class II molecule is inactivated.

The method of paragraph [084], wherein a gene regulating transcription of the MHC class II molecule is inactivated.

The method of paragraph [086], wherein the gene is CIITA.

The method of any one of paragraphs [069]-[087], wherein the exogenous MHC molecule comprises a MHC class I molecule, a MHC class II molecule, or a combination thereof, derived from the subject.

The method of paragraph [088], wherein the MHC class I molecule comprises HLA-A, HLA-B, HLA-C, or any combination thereof.

The method of paragraph or [089], wherein the MHC class II molecule comprises HLA-DP, HLA-DM, HLA-DOA, HLA-DOB, HLA-DQ, HLA-DR, or any combination thereof.

The method of any one of paragraphs [088]-[090], wherein the exogenous MHC molecule comprises an MHC-I alpha derived from the subject and an endogenous B2M.

The method of any one of paragraphs [088]-[090], wherein the exogenous MHC molecule comprises both an MHC-I alpha and a B2M derived from the subject.

The method of paragraph [092], wherein the exogenous MHC molecule is a fusion protein of the MHC-I alpha and the B2M (B2M-MHC-I-alpha fusion).

The method of paragraph [093], wherein the MHC-I alpha and the B2M is linked by a linker.

The method of paragraph [094], wherein the linker is (G4S)n, wherein G is glycine, S is serine, and n is an integer from 1 to 10.

The method of any one of paragraphs [088]-[095], wherein the exogenous MHC molecule comprises an MHC-II alpha and an MHC-II beta derived from the subject.

The method of any one of paragraphs [069]-[096], wherein the plurality of TCRs comprises V regions from a plurality of V genes.

The method of any one of paragraphs [069]-[097], wherein the plurality of TCRs is derived from a primary cell isolated from the same subject.

The method of paragraph [098], wherein the primary cell is a T cell.

The method of paragraph [099], wherein the T cell is a tumor-infiltrating T cell.

The method of paragraph [099], wherein the T cell is a peripheral T cell.

The method of paragraph [101], wherein the peripheral T cell is a tumor-experienced T cell.

The method of paragraph [101], wherein the peripheral T cell is a PD-1+ T cell.

The method of paragraph [099], wherein the T cell is a CD4+ T cell, a CD8+ T cell, or a combination thereof.

The method of paragraph [099], wherein the T cell is a cytotoxic T cell, a memory T cell, a national killer T cell, an alpha beta T cell, a gamma delta T cell, or any combination thereof.

The method of any one of paragraphs [069]-[105], wherein identifying in (c) comprises enriching or selecting the subset of the plurality of engineered cells.

The method of any one of paragraphs [069]-[106], wherein identifying in (c) comprises selecting the subset of the plurality of engineered cells based on a marker.

The method of paragraph [106], wherein selecting comprises using FACS or MACS based on the marker.

The method of paragraph or [108], wherein the marker is a reporter protein.

The method of paragraph [109], wherein the reporter protein is a fluorescent protein.

The method of paragraph or [108], wherein the marker is a cell surface protein, an intracellular protein or a secreted protein.

The method of paragraph [111], wherein the marker is the intracellular protein or the secreted protein, and wherein the method further comprises, prior to selecting, fixing and/or permeabilizing the plurality of engineered cells.

The method of paragraph [112], further comprising contacting the plurality of engineered cells with a Golgi blocker.

The method of any one of paragraphs [111]-[113], wherein the secreted protein is a cytokine.

The method of paragraph [114], wherein the cytokine is IFN-γ, TNF-alpha, IL-17A, IL-2, IL-3, IL-4, GM-CSF, IL-10, IL-13, granzyme B, perforin, or a combination thereof.

The method of any one of paragraphs [111]-[115], wherein the cell surface protein is CD39, CD69, CD103, CD25, PD-1, TIM-3, OX-40, 4-1BB, CD137, CD3, CD28, CD4, CD8, CD45RA, CD45RO, GITR, FoxP3, or a combination thereof.

The method of any one of paragraphs [069]-[116], further comprising identifying a TCR expressed by the antigen-reactive cell.

The method of paragraph [117], wherein identifying the TCR comprises sequencing a TCR repertoire of the subset of the plurality of engineered cells.

The method of any one of paragraphs [069]-[118], further comprising administering the antigen-reactive cell or a cell comprising a sequence encoding the TCR of the antigen-reactive cell into the subject.

The method of any one of paragraphs [069]-[119], further comprising, prior to (a), isolating a primary cancer cell from the subject.

The method of paragraph [120], further comprising conducting transcriptomic or genomic analysis of the primary cancer cell and cancer cell lines to identify the cancer cell line having a transcriptomic profile or genomic alteration that resembles a primary cancer cell isolated from the subject.

A pharmaceutical composition comprising an antigen-reactive cell or a cell comprising a sequence encoding a TCR of the antigen-reactive cell identified by a method of any one of paragraphs [001]-[121].

A composition for identifying an antigen-reactive cell that recognizes an endogenous antigen of a cancer cell line in complex with an MHC molecule expressed by a subject, comprising:
a cell that is a cancer cell line expressing an endogenous antigen in complex with an exogenous MHC molecule, wherein the exogenous MHC molecule is the MHC molecule expressed by the subject or derived from the subject; and
a T cell expressing a natively paired TCR derived from the subject, wherein a gene expression profile, a transcriptomic profile or a genomic alternation of the cancer cell line resembles that of a cancer cell from the subject.

The composition of paragraph [123], wherein a correlation coefficient of the gene expression profile, the transcriptomic profile or the genomic alteration between the cancer cell line and the primary cancer cell or the tumor sample is equal to or greater than about 0.1.

The composition of paragraph or [124], wherein the cancer cell line does not comprise or present an exogenous antigen.

The composition of any one of paragraphs [123]-[125], wherein an endogenous MHC molecule of the cancer cell line is inactivated.

The composition of any one of paragraphs [123]-[126], wherein the cancer cell line is null for an endogenous MHC molecule.

The composition of any one of paragraphs [123]-[127], wherein the cancer cell line is null for all endogenous MHC molecules.

The composition of any one of paragraphs [126]-[128], wherein the endogenous MHC molecule comprises a MHC class I molecule, a MEW class II molecule, or a combination thereof.

The composition of paragraph [129], wherein the MEW class I molecule comprises HLA-A, HLA-B, HLA-C, or any combination thereof.

The composition of paragraph or [130], wherein an alpha chain of the MEW class I molecule (MHC-I alpha) is inactivated.

The composition of paragraph [131], wherein a gene encoding the alpha chain of the MHC class I molecule is inactivated.

The composition of any one of paragraphs [129]-[132], wherein a beta-2-microglobulin (B2M) of the MEW class I molecule is inactivated.

The composition of paragraph [133], wherein a gene encoding the B2M of the MEW class I molecule is inactivated.

The composition of any one of paragraphs [129]-[134], wherein the MHC class II molecule comprises HLA-DP, HLA-DM, HLA-DOA, HLA-DOB, HLA-DQ, HLA-DR, or any combination thereof.

The composition of any one of paragraphs [129]-[135], wherein an alpha chain or a beta chain of the MEW class II molecule is inactivated.

The composition of paragraph [136], wherein a gene encoding the alpha chain or the beta chain of the MEW class II molecule is inactivated.

The composition of paragraph [136], wherein a gene regulating transcription of the WIC class II molecule is inactivated.

The composition of paragraph [138], wherein the gene is CIITA.

The composition of any one of paragraphs [123]-[139], wherein the exogenous MHC molecule of the cancer cell line comprises a MEW class I molecule, a MEW class II molecule, or a combination thereof, derived from the subject.

The composition of paragraph [140], wherein the MHC class I molecule comprises HLA-A, HLA-B, HLA-C, or any combination thereof.

The composition of paragraph or [141], wherein the MHC class II molecule comprises HLA-DP, HLA-DM, HLA-DOA, HLA-DOB, HLA-DQ, HLA-DR, or any combination thereof.

The composition of any one of paragraphs [140]-[142], wherein the exogenous MHC molecule comprises an MHC-I alpha derived from the subject and an endogenous B2M.

The composition of any one of paragraphs [140]-[143], wherein the exogenous MHC molecule comprises both an MHC-I alpha and a B2M derived from the subject.

The composition of paragraph [144], wherein the exogenous MHC molecule is a fusion protein of the MHC-I alpha and the B2M (B2M-MHC-I-alpha fusion).

The composition of paragraph [145], wherein the MHC-I alpha and the B2M is linked by a linker.

The composition of paragraph [146], wherein the linker is (G4S)n, wherein G is glycine, S is serine, and n is an integer from 1 to 10.

The composition of any one of paragraphs [140]-[147], wherein the exogenous MHC molecule comprises an MHC-II alpha and an MHC-II beta derived from the subject.

The composition of any one of paragraphs [123]-[148], wherein the T cell are a plurality of T cells, each expressing a different natively paired TCR derived from the subject.

The composition of paragraph [149], wherein the plurality of T cells comprise at least 10 different natively paired TCRs derived from the subject.

A method for evaluating an anti-cancer activity of a TCR-expressing cell, comprising:
(a) providing a plurality of cells, wherein the plurality of cells is derived from a cancer cell line and expresses an endogenous antigen in complex with an exogenous MHC molecule, wherein the exogenous MHC molecule is an MHC molecule expressed by a subject or derived from the subject;
(b) contacting the plurality of cells with a plurality of TCR-expressing cells expressing a plurality of TCRs derived from the same subject, wherein the plurality of TCRs or a fraction thereof recognizes the endogenous antigen in complex with the exogenous MHC molecule of the plurality of cells or a fraction thereof; and
(c) subsequent to contacting in (b), quantifying (i) the fraction of the plurality of cells that are recognized by the plurality of TCR-expressing cells or a fraction thereof, (ii) the fraction of the plurality of TCR-expressing cells that recognize the plurality of cells or a fraction thereof, and/or (iii) an amount or level of a cytokine secreted by the plurality of TCR-expressing cells or a fraction thereof.

The method of paragraph [151], wherein an endogenous MEW molecule of the plurality of cells is inactivated.

The method of paragraph or [152], wherein the plurality of cells is null for an endogenous MHC molecule.

The method of any one of paragraphs [151]-[153], wherein the plurality of cells is null for all endogenous MHC molecules.

The method of any one of paragraphs [151]-[154], wherein the endogenous MEW molecule comprises a MHC class I molecule, a MEW class II molecule, or a combination thereof.

The method of paragraph [155], wherein an alpha chain of the MEW class I molecule (MHC-I alpha) is inactivated.

The method of paragraph [156], wherein a gene encoding the alpha chain of the MEW class I molecule is inactivated.

The method of any one of paragraphs [155]-[157], wherein a beta-2-microglobulin (B2M) of the MHC class I molecule is inactivated.

The method of paragraph [158], wherein a gene encoding the B2M of the MEW class I molecule is inactivated.

The method of any one of paragraphs [155]-[159], wherein an alpha chain or a beta chain of the MHC class II molecule is inactivated.

The method of paragraph [160], wherein a gene encoding the alpha chain or the beta chain of the MHC class II molecule is inactivated.

The method of paragraph or [161], wherein a gene regulating transcription of the MEW class II molecule is inactivated.

The method of any one of paragraphs [151]-[162], wherein the exogenous MHC molecule of the plurality of cells comprises a MEW class I molecule, a MEW class II molecule, or a combination thereof, derived from the subject.

The method of paragraph [163], wherein the exogenous MEW molecule comprises an MHC-I alpha derived from the subject and an endogenous B2M.

The method of paragraph [163], wherein the exogenous MEW molecule comprises both an MHC-I alpha and a B2M derived from the subject.

The method of paragraph [165], wherein the exogenous MEW molecule is a fusion protein of the MHC-I alpha and the B2M (B2M-MHC-I-alpha fusion).

The method of paragraph [166], wherein the MHC-I alpha and the B2M is linked by a linker.

The method of any one of paragraphs [151]-[167], wherein the exogenous MHC molecule comprises an WIC-II alpha and an MHC-II beta derived from the subject.

The method of any one of paragraphs [151]-[168], wherein the plurality of TCR-expressing cells is isolated from the same subject.

The method of any one of paragraphs [151]-[169], wherein the plurality of TCR-expressing cells comprises a primary T cell.

The method of any one of paragraphs [151]-[168], wherein the plurality of TCR-expressing cells comprises an engineered cell.

The method of paragraph [171], wherein the engineered cell expresses an exogenous TCR.

The method of any one of paragraphs [151]-[172], wherein quantifying the fraction of (i) or (ii) comprising using a flow cytometry based method.

The method of paragraph [173], wherein the flow cytometry based method is FACS or MACS.

The method of any one of paragraphs [151]-[172], wherein quantifying the fraction of (i) comprising determining an amount of lactate dehydrogenase released from the fraction.

A composition comprising a panel of MHC-engineered cancer cell lines derived from a same cancer type, comprising:
a first sub-panel comprising at least two WIC-engineered cancer cell lines derived from a same first parental cancer cell line; and
a second sub-panel comprising at least two WIC-engineered cancer cell lines derived from a same second parental cancer cell line; and
wherein the at least two MHC-engineered cancer cell lines of the first sub-panel or the second sub-panel expresses a different exogenous WIC molecule.

The composition of paragraph [176], wherein the at least two WIC-engineered cancer cell lines of the first sub-panel or the second sub-panel do not express a same exogenous and/or endogenous MHC molecule.

The composition of paragraph or [177], wherein the at least two MHC-engineered cancer cell lines comprise at least about 10, 20, 30, 40, 50, 60, 70, 80, 90, 100, or more WIC-engineered cancer cell lines, each MHC-engineered cancer cell line expressing a different exogenous MHC molecule.

The composition of any one of paragraphs [176]-[178], wherein the first parental cancer cell line and the second parental cancer cell line are different.

The composition of any one of paragraphs [176]-[179], wherein an endogenous MHC molecule of the at least two MHC-engineered cancer cell lines of the first sub-panel or the second sub-panel is inactivated.

The composition of any one of paragraphs [176]-[180], wherein the exogenous MHC molecule is expressed by a subject or derived from the subject.

The composition of any one of paragraphs [176]-[181], wherein the composition further comprises a plurality of T cells.

The composition of paragraph [182], wherein each cancer cell line of the at least two MHC-engineered cancer cell lines in the first sub-panel or the second sub-panel is mixed with the plurality of T cells.

The composition of paragraph or [183], wherein the plurality of T cells comprises at least two different natively paired TCRs.

The composition of paragraph [184], wherein the natively paired TCRs are derived from the same subject.

The composition of any one of paragraphs [176]-[185], wherein the panel of MHC-engineered cancer cell lines is derived from bladder cancer, bone cancer, brain cancer, breast cancer, colon cancer, ovarian cancer, head/neck cancer, leukemia, lymphoma, liver cancer, lung cancer, melanoma, pancreatic cancer, soft-tissue sarcoma, or stomach cancer.

A method for identifying an antigen-reactive cell that recognizes an endogenous antigen in complex with an MHC molecule expressed by a subject, the method comprising:
(a) providing an antigen-presenting cell (APC) expressing an endogenous antigen in complex with an exogenous MHC molecule, wherein the exogenous MHC molecule is the MHC molecule expressed by the subject or derived from the subject;
(b) contacting the APC with a plurality of TCR-expressing cells derived from the subject, wherein the plurality of TCR-expressing cells or a subset of the plurality of TCR-expressing cells recognizes the endogenous antigen in complex with the exogenous MHC of the APC, and wherein the plurality of TCR-expressing cells or a subset of the plurality of TCR-expressing cells that recognizes the endogenous antigen (i) is attached to a label secreted from the APC or a label transferred by a label-transferring enzyme associated with the APC upon recognizing the endogenous antigen, or (ii) expresses an activation marker upon recognizing the endogenous antigen; and
(c) identifying the subset of the plurality of TCR-expressing cells based on the label or the activation marker, thereby identifying the antigen-reactive cell.

The method of paragraph [187], wherein identifying comprises enriching the subset of the plurality of TCR-expressing cells.

The method of paragraph or [188], wherein the APC expresses at least about 100 endogenous antigens.

The method of paragraph [187], wherein the method further comprises determining whether to administer a cancer drug to the subject based on a fraction of the subset of the plurality of TCR-expressing cells in the plurality of TCR-expressing cells or the number of the TCR-expressing cells in the subset.

The method of any one of paragraphs [187]-[189], further comprising quantifying the number of the subset of the plurality of TCR-expressing cells.

The method of paragraph [191], further comprising quantifying the number of the plurality of TCR-expressing cells prior to contacting in (b).

The method of paragraph [192], further comprising determining a fraction of the subset of the plurality of TCR-expressing cells in the plurality of TCR-expressing cells.

The method of paragraph 163 or [193], further comprising determining whether to administer a cancer drug to the subject based on the fraction or the number of the TCR-expressing cells in the subset.

The method of paragraph [194], further comprising administering a cancer drug to the subject determined as being suitable for treatment with the cancer drug based on the fraction.

The method of paragraph [194], further comprising not administering a cancer drug to the subject determined as being unsuitable for treatment with the cancer drug based on the fraction.

The method of paragraph or [195], further comprising increasing a dose of the cancer drug to the subject.

The method of paragraph or [195], further comprising decreasing a dose of the cancer drug to the subject.

The method of any one of paragraphs [194]-[198], wherein the cancer drug is an immune cell regulator.

The method of paragraph [199], wherein the immune cell regulator is a cytokine or an immune checkpoint inhibitor.

The method of any one of paragraphs [187]-[200], further comprising determining a TCR sequence of the subset of the plurality of TCR-expressing cells.

The method of paragraph [201], further comprising delivering a polynucleotide molecule having the TCR sequence into a recipient cell for expression.

The method of paragraph [202], wherein the recipient cell does not comprise the TCR sequence prior to delivering.

The method of paragraph [203], wherein an endogenous TCR of the recipient cell is inactivated.

The method of any one of paragraphs [202]-[204], wherein the recipient cell is a T cell.

The method of paragraph [205], wherein the T cell is an autologous T cell or an allogenic T cell.

The method of any one of paragraphs [202]-[206], further comprising administering the recipient cell or derivative thereof into the subject.

The method of any one of paragraphs [188]-[207], wherein the subset of the plurality of TCR-expressing cells expresses at least two different TCRs.

The method of paragraph [208], further comprising determining sequences of the at least two different TCRs.

The method of paragraph [209], further comprising delivering a plurality of polynucleotide molecules encoding the at least two different TCRs into a plurality of recipient cells for expression.

The method of paragraph [210], further comprising contacting the plurality of recipient cells with the APC or an additional APC.

The method of paragraph [211], further comprising enriching a recipient cell from the plurality of recipient cells, which recipient cell recognizes the APC or the additional APC.

The method of any one of paragraphs [187]-[212], wherein the label comprises a detectable moiety, which detectable moiety is detectable by flow cytometry.

The method of paragraph [213], wherein the detectable moiety is a biotin, a fluorescent dye, a peptide, digoxigenin, or a conjugation handle.

The method of paragraph [214], wherein the conjugation handle comprises an azide, an alkyne, a DBCO, a tetrazine, or a TCO.

The method of any one of paragraphs [187]-[215], wherein the label comprises a substrate recognized by the label-transferring enzyme.

The method of any one of paragraphs [187]-[216], wherein the label is a cytokine secreted by the APC.

The method of any one of paragraphs [187]-[217], wherein the label-transferring enzyme is a transpeptidase or a glycosyltransferase.

The method of paragraph [218], wherein the transpeptidase is a sortase.

The method of paragraph [219], wherein the glycosyltransferase is a fucosyltransferase.

The method of any one of paragraphs [187]-[220], wherein the label-transferring enzyme is expressed by the APC or is supplied outside and attached to the APC.

The method of paragraph [221], wherein the label-transferring enzyme is a transmembrane protein.

The method of paragraph [221], wherein the label-transferring enzyme is attached to the APC via covalent or non-covalent interaction.

The method of any one of paragraphs [187]-[223], wherein the APC is derived from a subject.

The method of any one of paragraphs [187]-[224], wherein the APC is a cancer cell line.

The method of any one of paragraphs [187]-[225], wherein the subject has cancer.

The method of paragraph [226], wherein the cancer cell line is derived from a same cancer type as the cancer of the subject.

The method of any one of paragraphs [187]-[225], wherein the plurality of TCR-expressing cells comprises T cells.

The method of paragraph [228], wherein the T cells are tumor-infiltrating T cells or peripheral T cells.

The method of paragraph [229], wherein the T cells express LAG3, CD39, CD69, CD103, CD25, PD-1, TIM-3, OX-40, 4-1BB, CD137, CD3, CD28, CD4, CD8, CD45RA, CD45RO, GITR, FoxP3, or any combinations thereof.

The method of any one of paragraphs [187]-[230], wherein the plurality of TCR-expressing cells comprises a label-accepting moiety, which label-accepting moiety receives the label.

A pharmaceutical composition comprising an antigen-reactive cell or a cell comprising a sequence encoding a TCR of the antigen-reactive cell identified by a method of any one of paragraphs [187]-[231].

What is claimed is:

1. A method for identifying an antigen-reactive cell that recognizes an endogenous antigen of a cancer cell line in complex with an MHC molecule expressed by a subject, comprising:
(a) providing a cell that is a cancer cell line expressing an endogenous antigen in complex with an exogenous MHC molecule, wherein the exogenous MHC molecule is the MHC molecule expressed by the subject or derived from the subject;
(b) contacting the cancer cell line with a first plurality of TCR-expressing cells, wherein the first plurality of TCR-expressing cells or a subset of the first plurality of TCR-expressing cells is activated by the endogenous antigen in complex with the exogenous MHC of the cancer cell line; and
(c) subsequent to contacting in (b), identifying the subset of the first plurality of TCR-expressing cells, thereby identifying the antigen-reactive cell that recognizes the endogenous antigen of the cancer cell line.

2. The method of claim 1, wherein identifying in (c) comprises enriching or selecting the subset of the first plurality of TCR-expressing cells.

3. The method of claim 1 or 2, wherein the exogenous MHC molecule is exogenous to the cancer cell line.

4. The method of claim 1, wherein the method further comprises, in (a), providing a non-cancer cell expressing an additional endogenous antigen in complex with an exogenous MHC molecule, wherein the exogenous MHC molecule is derived from the same subject.

5. The method of claim 4, further comprising, in (b), contacting the non-cancer cell with a second plurality of TCR-expressing cells, and wherein a subset of the second plurality of TCR-expressing cells is activated by the additional endogenous antigen in complex with the exogenous MHC of the non-cancer cell.

6. The method of claim 4 or 5, wherein the additional endogenous antigen is the same as or different from the endogenous antigen expressed by the cancer cell line.

7. The method of any one of claims 4-6, wherein the non-cancer cell (i) does not express the endogenous antigen expressed by the cancer cell line, (ii) expresses the endogenous antigen expressed by the cancer cell line at a lower level, or (iii) expresses the endogenous antigen expressed by the cancer cell line, but does not present the endogenous antigen expressed by the cancer cell line.

8. The method of any one of claims 5-7, wherein the first plurality and the second plurality of TCR-expressing cells are derived from a same sample.

9. The method of any one of claims 5-8, wherein the first plurality and the second plurality of TCR-expressing cells express a same TCR.

10. The method of any one of claims 5-9, wherein the first plurality or the second plurality of TCR-expressing cells expresses different TCRs.

11. The method of any one of claims 5-10, further comprising, in (c), identifying the subset of the second plurality of TCR-expressing cells.

12. The method of any one of claims 1-11, wherein identifying comprises selecting the subset of the first plurality of TCR-expressing cells and/or the subset of the second plurality of TCR-expressing cells based on a marker.

13. The method of claim 12, wherein selecting the subset of the first plurality of TCR-expressing cells and/or the subset of the second plurality of TCR-expressing cells comprises using fluorescence activated cell sorting (FACS) or magnetic activated cell sorting (MACS) based on the marker.

14. The method of claim 13, further comprising identifying a TCR that is expressed in the subset of the first plurality of TCR-expressing cells.

15. The method of claim 13, further comprising identifying a TCR that is expressed in the subset of the first plurality of TCR-expressing cells, but not in the subset of the second plurality of TCR-expressing cells.

16. The method of claim 13, further comprising identifying a TCR of a cell in the subset of the first plurality of TCR-expressing cells that is activated by the endogenous antigen in complex with the exogenous MHC of the cancer cell line, and that is in a cell in the second plurality of TCR-expressing cells that is not activated by the additional endogenous antigen in complex with the exogenous MHC of the non-cancer cell.

17. The method of any one of claims 4-16, wherein the non-cancer cell is a stem cell or a primary cell.

18. The method of claim 17, wherein the stem cell is an induced pluripotent stem cell (iPSC).

19. The method of claim 18, wherein the non-cancer cell is an differentiated iPSC.

20. The method of any one of claims 4-19, wherein the non-cancer cell expresses an autoimmune regulator (AIRE).

21. The method of any one of claims 1-19, wherein an endogenous MHC molecule of the cancer cell line or the non-cancer cell is inactivated (e.g., knocked down, or knocked out).

22. The method of any one of claims 1-21, wherein the cancer cell line or non-cancer cell is null for an endogenous MEW molecule.

23. The method of any one of claims 1-22, wherein the cancer cell line or non-cancer cell is null for all endogenous MEW molecules.

24. The method of any one of claims 21-23, wherein the endogenous MHC molecule comprises a MHC class I molecule, a MHC class II molecule, or a combination thereof.

25. The method of claim 24, wherein the MEW class I molecule comprises HLA-A, HLA-B, HLA-C, or any combination thereof.

26. The method of claim 24 or 25, wherein an alpha chain of the MEW class I molecule (MHC-I alpha) is inactivated.

27. The method of claim 26, wherein a gene encoding the alpha chain of the MHC class I molecule is inactivated.

28. The method of any one of claims 24-27, wherein a beta-2-microglobulin (B2M) of the MEW class I molecule is inactivated.

29. The method of claim 28, wherein a gene encoding the B2M of the MEW class I molecule is inactivated.

30. The method of any one of claims 24-29, wherein the MHC class II molecule comprises HLA-DP, HLA-DM, HLA-DOA, HLA-DOB, HLA-DQ, HLA-DR, or any combination thereof.

31. The method of any one of claims 24-30, wherein an alpha chain or a beta chain of the MEW class II molecule is inactivated.

32. The method of claim 31, wherein a gene encoding the alpha chain or the beta chain of the MEW class II molecule is inactivated.

33. The method of claim 31, wherein a gene regulating transcription of the MHC class II molecule is inactivated.

34. The method of claim 33, wherein the gene is CIITA.

35. The method of any one of claims 1-34, wherein the exogenous MHC molecule of the cancer cell line or the non-cancer cell comprises a MEW class I molecule, a MEW class II molecule, or a combination thereof, derived from the subject.

36. The method of claim 35, wherein the MEW class I molecule comprises HLA-A, HLA-B, HLA-C, or any combination thereof.

37. The method of claim 35 or 36, wherein the MHC class II molecule comprises HLA-DP, HLA-DM, HLA-DOA, HLA-DOB, HLA-DQ, HLA-DR, or any combination thereof.

38. The method of any one of claims 35-37, wherein the exogenous MHC molecule comprises an MHC-I alpha derived from the subject and an endogenous B2M.

39. The method of any one of claims 35-38, wherein the exogenous MHC molecule comprises both an MHC-I alpha and a B2M derived from the subject.

40. The method of claim 39, wherein the exogenous MHC molecule is a fusion protein of the MHC-I alpha and the B2M (B2M-MHC-I-alpha fusion).

41. The method of claim 40, wherein the MHC-I alpha and the B2M is linked by a linker.

42. The method of claim 41, wherein the linker is (G₄S)ₙ, wherein G is glycine, S is serine, and n is an integer from 1 to 10.

43. The method of any one of claims 35-42, wherein the exogenous MHC molecule comprises an MHC-II alpha and an MHC-II beta derived from the subject.

44. The method of any one of claims 1-43, wherein the plurality of TCR-expressing cells is isolated from the same subject.

45. The method of any one of claims 1-44, wherein the plurality of TCR-expressing cells comprises a primary T cell.

46. The method of claim 45, wherein the primary T cell is a tumor-infiltrating T cell.

47. The method of claim 45, wherein the primary T cell is a peripheral T cell.

48. The method of claim 47, wherein the peripheral T cell is a tumor-experienced T cell.

49. The method of claim 47, wherein the peripheral T cell is a PD-1⁺ T cell.

50. The method of any one of claims 45-49, wherein the primary T cell is a CD4⁺ T cell, a CD8⁺ T cell, or a combination thereof.

51. The method of any one of claims 45-49, wherein the primary T cell is a cytotoxic T cell, a memory T cell, a national killer T cell, an alpha beta T cell, a gamma delta T cell, or any combination thereof.

52. The method of any one of claims 1-51, wherein the plurality of TCR-expressing cells comprises an engineered cell.

53. The method of claim 52, wherein the engineered cell expresses an exogenous TCR.

54. The method of claim 53, wherein the exogenous TCR is derived from a primary T cell isolated from the same subject.

55. The method of any one of claims 1-54, further comprising, prior to (a), isolating a primary cancer cell or a tumor sample from the subject.

56. The method of claim 55, further comprising conducting transcriptomic or genomic analysis of the primary cancer cell or the tumor sample and cancer cell lines to identify the cancer cell line having a gene expression profile, a transcriptomic profile or a genomic alteration that resembles a primary cancer cell or the tumor sample isolated from the subject.

57. The method of claim 56, wherein a correlation coefficient of the gene expression profile, the transcriptomic profile or the genomic alteration between the cancer cell line and the primary cancer cell or the tumor sample is equal to or greater than about 0.1.

58. The method of any one of claims 1-57, further comprising, in (c), identifying a TCR of the subset.

59. The method of claim 58, further comprising identifying a sequence of a TCR expressed by the antigen-reactive cell.

60. The method of claim 59, wherein identifying the sequence of the TCR comprises sequencing a TCR repertoire of the subset of the first plurality of TCR-expressing cells.

61. The method of claim 60, wherein identifying the sequence of the TCR further comprises sequencing a TCR repertoire of the first plurality of TCR-expressing cells prior to contacting with the cancer cell line.

62. The method of claim 61, wherein a frequency of the TCR expressed by the antigen-reactive cell in the subset is higher than a frequency of the TCR expressed by the antigen-reactive cell in the first plurality.

63. The method of any one of claims 1-62, further comprising administering the antigen-reactive cell or a cell comprising a sequence encoding the TCR of the antigen-reactive cell into the subject.

64. The method of any one of claims 1-63, wherein the first plurality of TCR-expressing cells expresses a plurality of TCRs comprising at least 10 different cognate pairs derived from the same subject.

65. The method of claim 64, wherein the plurality of TCRs comprises V regions from a plurality of V genes.

66. The method of any one of claims 1-65, wherein the cell that is a cancer cell line comprises at least about 50, 100, 1,000 or more cells.

67. The method of any one of claims 1-66, further comprising, prior to (b), killing the cancer cell line.

68. The method of claim 67, wherein killing comprising irradiating or treating the cancer cell line with a chemical compound.

69. The method of claim 68, wherein the chemical compound is a cytotoxic compound.

70. The method of claim 69, wherein the cytotoxic compound is cis-platin, cyclophosphamide, nitrogen mustard, trimethylene thiophosphoramide, carmustine, busulfan, chlorambucil, belustine, uracil mustard, chlomaphazin, dacabazine, cytosine arabinoside, fluorouracil, methotrexate, mercaptopuirine, azathioprime, procarbazine, doxorubicin, bleomycin, dactinomycin, daunorubicin, mithramycin, mitomycin, mytomycin C, daunomycin, or any combination thereof.

71. A pharmaceutical composition comprising an antigen-reactive cell or a cell comprising a sequence encoding a TCR of the antigen-reactive cell identified by a method of any one of claims 1-70.

72. A composition for identifying an antigen-reactive cell that recognizes an endogenous antigen of a cancer cell line in complex with an MHC molecule expressed by a subject, comprising:
a cell that is a cancer cell line expressing an endogenous antigen in complex with an exogenous MHC molecule, wherein the exogenous MHC molecule is the MEW molecule expressed by the subject or derived from the subject; and
a T cell expressing a natively paired TCR derived from the subject, wherein a gene expression profile, a transcriptomic profile or a genomic alternation of the cancer cell line resembles that of a cancer cell from the subject.

73. The composition of claim 72, wherein a correlation coefficient of the gene expression profile, the transcriptomic profile or the genomic alteration between the cancer cell line and the primary cancer cell or the tumor sample is equal to or greater than about 0.1.

74. The composition of claim 72 or 73, wherein the cancer cell line does not comprise or present an exogenous antigen.

75. The composition of any one of claims 72-74, wherein an endogenous MHC molecule of the cancer cell line is inactivated.

76. The composition of any one of claims 72-75, wherein the cancer cell line is null for an endogenous MHC molecule.

77. The composition of any one of claims 72-76, wherein the cancer cell line is null for all endogenous MHC molecules.

78. The composition of any one of claims 75-77, wherein the endogenous MHC molecule comprises a MHC class I molecule, a MHC class II molecule, or a combination thereof.

79. The composition of claim 78, wherein the MEW class I molecule comprises HLA-A, HLA-B, HLA-C, or any combination thereof.

80. The composition of claim 78 or 79, wherein an alpha chain of the MEW class I molecule (MHC-I alpha) is inactivated.

81. The composition of claim 80, wherein a gene encoding the alpha chain of the MEW class I molecule is inactivated.

82. The composition of any one of claims 78-81, wherein a beta-2-microglobulin (B2M) of the MEW class I molecule is inactivated.

83. The composition of claim 82, wherein a gene encoding the B2M of the MEW class I molecule is inactivated.

84. The composition of any one of claims 78-83, wherein the MEW class II molecule comprises HLA-DP, HLA-DM, HLA-DOA, HLA-DOB, HLA-DQ, HLA-DR, or any combination thereof.

85. The composition of any one of claims 78-84, wherein an alpha chain or a beta chain of the MEW class II molecule is inactivated.

86. The composition of claim 85, wherein a gene encoding the alpha chain or the beta chain of the MHC class II molecule is inactivated.

87. The composition of claim 85, wherein a gene regulating transcription of the MHC class II molecule is inactivated.

88. The composition of claim 87, wherein the gene is CIITA.

89. The composition of any one of claims 72-88, wherein the exogenous MHC molecule of the cancer cell line comprises a MHC class I molecule, a MHC class II molecule, or a combination thereof, derived from the subject.

90. The composition of claim 89, wherein the MEW class I molecule comprises HLA-A, HLA-B, HLA-C, or any combination thereof.

91. The composition of claim 89 or 90, wherein the MEW class II molecule comprises HLA-DP, HLA-DM, HLA-DOA, HLA-DOB, HLA-DQ, HLA-DR, or any combination thereof.

92. The composition of any one of claims 89-91, wherein the exogenous MHC molecule comprises an MHC-I alpha derived from the subject and an endogenous B2M.

93. The composition of any one of claims 89-92, wherein the exogenous MHC molecule comprises both an MHC-I alpha and a B2M derived from the subject.

94. The composition of claim 93, wherein the exogenous MEW molecule is a fusion protein of the MHC-I alpha and the B2M (B2M-MHC-I-alpha fusion).

95. The composition of claim 94, wherein the MHC-I alpha and the B2M is linked by a linker.

96. The composition of claim 95, wherein the linker is (G₄S)ₙ, wherein G is glycine, S is serine, and n is an integer from 1 to 10.

97. The composition of any one of claims 89-96, wherein the exogenous MHC molecule comprises an MHC-II alpha and an WIC-II beta derived from the subject.

98. The composition of any one of claims 72-97, wherein the T cell are a plurality of T cells, each expressing a different natively paired TCR derived from the subject.

99. The composition of claim 98, wherein the plurality of T cells comprise at least 10 different natively paired TCRs derived from the subject.

100. A method for evaluating an anti-cancer activity of a TCR-expressing cell, comprising:
(a) providing a plurality of cells, wherein the plurality of cells is derived from a cancer cell line and expresses an endogenous antigen in complex with an exogenous WIC molecule, wherein the exogenous MHC molecule is an WIC molecule expressed by a subject or derived from the subject;
(b) contacting the plurality of cells with a plurality of TCR-expressing cells expressing a plurality of TCRs derived from the same subject, wherein the plurality of TCRs or a fraction thereof recognizes the endogenous antigen in complex with the exogenous MHC molecule of the plurality of cells or a fraction thereof; and
(c) subsequent to contacting in (b), quantifying (i) the fraction of the plurality of cells that are recognized by the plurality of TCR-expressing cells or a fraction thereof, (ii) the fraction of the plurality of TCR-expressing cells that recognize the plurality of cells or a fraction thereof, and/or (iii) an amount or level of a cytokine secreted by the plurality of TCR-expressing cells or a fraction thereof.

101. A composition comprising a panel of WIC-engineered cancer cell lines derived from a same cancer type, comprising:
a first sub-panel comprising at least two MHC-engineered cancer cell lines derived from a same first parental cancer cell line; and
a second sub-panel comprising at least two MHC-engineered cancer cell lines derived from a same second parental cancer cell line; and
wherein the at least two MHC-engineered cancer cell lines of the first sub-panel or the second sub-panel expresses a different exogenous MHC molecule.

102. The composition of claim 101, wherein the at least two WIC-engineered cancer cell lines of the first sub-panel or the second sub-panel do not express a same exogenous and/or endogenous MHC molecule.

103. A method for identifying an antigen-reactive cell that recognizes an endogenous antigen in complex with an WIC molecule expressed by a subject, the method comprising:
(a) providing an antigen-presenting cell (APC) expressing an endogenous antigen in complex with an exogenous MHC molecule, wherein the exogenous MHC molecule is the MHC molecule expressed by the subject or derived from the subject;
(b) contacting the APC with a plurality of TCR-expressing cells derived from the subject, wherein the plurality of TCR-expressing cells or a subset of the plurality of TCR-expressing cells recognizes the endogenous antigen in complex with the exogenous MHC of the APC, and wherein the plurality of TCR-expressing cells or a subset of the plurality of TCR-expressing cells that recognizes the endogenous antigen (i) is attached to a label secreted from the APC or a label transferred by a label-transferring enzyme associated with the APC upon recognizing the endogenous antigen, or (ii) expresses an activation marker upon recognizing the endogenous antigen; and
(c) identifying the subset of the plurality of TCR-expressing cells based on the label or the activation marker, thereby identifying the antigen-reactive cell.
